(19) United States
(12) Patent Application Publication
(10) Pub. No.: US 2008/0038244 A1
(43) Pub. Date: Feb. 14, 2008

(54) Materials and Methods Relating to Cell Cycle Control

(75) Inventors: David Glover, Great Gransden (GB); Monica Bettencourt-Dias, Lisboa (PT); Regis Giet, Marcille-Raoul (FR); Rita Sinka, Cambridge (GB); Lee Carpenter, Stowesfield (GB)

Correspondence Address: DANN, DORFMAN, HERRELL & SKILLMAN, 1601 MARKET STREET, SUITE 2400, PHILADELPHIA, PA, 19103-2307, US

(73) Assignee: CANCER RESEARCH TECHNOLOGY LTD, London (GB)

(21) Appl. No.: 10/582,446

(22) PCT Filed: Dec. 13, 2004
(86) PCT No.: PCT/GB04/05218

§ 371 Date: Jun. 11, 2007

(30) Foreign Application Priority Data
Dec. 12, 2003 (GB) 0328928.7

(51) Int. Cl. A61K 31/7088; A61K 38/45; A61P 17/06; A61P 35/00; C12N 15/63; C12N 9/12; C12Q 1/48; C12Q 1/68; G01N 33/53

(52) U.S. Cl. 424/94.5; 435/15; 435/194; 435/320.1; 435/6; 435/7.1; 514/44

(57) ABSTRACT

A screen using RNAi methods was used to test the entire set of protein kinases in Drosophila for an effect on mitosis. Most kinases previously known to be involved in the cell cycle were identified, providing validation of the approach. A mitotic function was found for a number of kinases not previously known to be involved in the cell cycle. Materials and methods are therefore provided for control of the cell cycle using modulators of expression or activity of kinases not previously known to act in mitosis, including human orthologues thereof.

### FIELD OF THE INVENTION

The present invention relates to materials and methods for cell cycle control, and in particular to materials and methods for modulating the activity of kinases which play a role in regulation of the cell cycle. Specifically, the present invention identifies kinases which were not previously known to be involved in cell cycle regulation, and provides methods and compositions for control of the cell cycle using agents capable of modulating the activity or expression of these kinases. Also provided are methods for identification of such agents, as well as their use in control of the cell cycle, including therapeutic use in control of proliferative disease.

### BACKGROUND TO THE INVENTION

Mitosis is a highly dynamic process that depends on networks of protein phosphorylation and dephosphorylation. Much of our insight on the roles of protein kinases in mitosis has come from the study of mutations in genetically tractable organisms. However, the use of classical genetics to study mitosis in metazoans is limited and does not permit full genome coverage. The availability of a fully sequenced and annotated genome, combined with the use of double stranded RNA mediated interference (RNAi) in D. melanogaster tissue culture cells, has made possible the exploration of that part of the genome not easily amenable to classical genetic studies (Clemens et al., 2000; Giet and Glover, 2001; Giet et al., 2002)(Goshima and Vale, 2003; Kiger et al., 2003; Lum et al., 2003; Rogers et al., 2003; Somma et al., 2002). The drosophila kinome shows little redundancy: drosophila only has 239 protein kinases as compared to 454 in worms (Manning, 2002) and 518 in humans (Manning, 2002b). Additionally, all subfamilies of protein kinases present in flies are also represented in the human genome (Manning, 2002). Here we describe a screen to test the entire set of Drosophila protein kinases for a function in mitosis. In this screen we have used FACS analysis to identify changes in the progression through the cell cycle, and to check for aneuploidy, polyploidy and cell death. Visualization of centrosomes, microtubules and DNA by immunocytochemistry has enabled the quantitation of multiple cell cycle parameters: mitotic index; percentages of cells in different phases of mitosis; defects in duplication, maturation and separation of centrosomes; abnormalities of condensation and segregation of chromosomes; and defects in spindle assembly and cytokinesis.

### SUMMARY OF THE INVENTION

It has been known for many years that a number of protein kinases are important in regulation of the eukaryotic cell cycle. By screening Drosophila cells with a protocol utilising RNAi, the present inventors have now identified roles in the cell cycle for a set of protein kinases not previously known to be involved in cell cycle control.

In a first aspect, the present invention provides a method of modulating proliferation in a cell or population of cells, comprising contacting said cell or population of cells with an agent capable of modulating expression or activity of a target kinase of Table 1 or Table 2. Table 2 shows Drosophila kinases identified by the screening protocol as being implicated in the control of the cell cycle. Table 1 shows a preferred subset of these kinases, along with human orthologues of these genes. Reference to a target kinase of Table 1 should be taken to mean the human sequence unless otherwise specified.

Table 1 also includes a small number of proteins which, while not kinases themselves, bind to kinases of table 1 and regulate their activities. For example, association between the kinase and the regulator may be required for kinase activity, or may increase kinase activity. Examples of such regulators are shown in FIG. 6 and include SNF4γ, which regulates SNF1A. Thus, for simplicity, reference will be made throughout this specification to kinases of Table 1, but this should be taken to include regulator molecules of Table 1.

The method may be performed in vitro. However the invention also extends to the in vivo administration of such agents.

In a further aspect, the present invention provides a method of screening for a modulator of cell proliferation, comprising determining the effect of a candidate substance on the expression or activity of a target kinase of Table 1.

The method may comprise the step of contacting a cell capable of expressing the target kinase with the candidate substance. The cell may be capable of expressing the target kinase from an endogenous coding sequence, or from an exogenous coding sequence introduced to the cell via a suitable vector.

Alternatively the method may comprise contacting the target kinase protein directly with the candidate substance, e.g. in a cell-free system.

The method will typically comprise the step of determining the level of expression or activity of the target kinase.

The method may further comprise the step of determining the effect of the candidate substance on proliferation (e.g. division) of a cell or population of cells.

The method may further comprise determining the extent to which apoptosis occurs in the cell or population of cells. This may be performed by analysing fragmentation of genomic DNA, TUNEL assay, or any other appropriate assay.

The candidate substance may be a nucleic acid, a protein, polypeptide, peptide or small molecule.

In a further aspect, the present invention provides a method of determining the effect of a candidate substance on proliferation of a cell or population of cells, comprising contacting said cell or population of cells with said candidate substance, said candidate substance having previously been identified as a modulator of activity or expression of a target kinase of Table 1.

This aspect of the invention thus extends to agents already known to modulate activity or expression of the target kinase, but which were not previously appreciated to be capable of exerting an effect on the cell cycle via this modulatory activity, as well as modulators identified by the methods described above.

The target kinases of the present invention may be suitable therapeutic targets for treatment of a proliferative disorder, as described in more detail below.

Thus the invention further provides a method of preparing a pharmaceutical composition, preferably for the treatment of a proliferative disorder, the method comprising, having identified a modulator of proliferation or of target kinase activity (e.g. by the above-described methods), formulating said modulator with a pharmaceutically acceptable carrier.

The method may further comprise the preliminary step of optimising the modulator for in vivo administration.

The term “proliferative disorder” encompasses cancer, psoriasis, glomerulonephritis and any other disorder characterised by abnormal cellular proliferation.

A further aspect of the invention relates to the use of a modulator of a target kinase of Table 1 for the inhibition of cell proliferation, preferably for the treatment of a proliferative disorder. The invention therefore provides a method of treatment of a proliferative disorder in a subject suffering therefrom, comprising administering to said subject a modulator of a target kinase of Table 1. Also provided is the use of a modulator of a target kinase of Table 1 in the manufacture of a medicament for the inhibition of cell proliferation, preferably for the treatment of a proliferative disorder.

It is envisaged that the target kinases of the present invention may also be used as markers for proliferative disease. Therefore the present invention further provides a method of diagnosis of a proliferative disorder, comprising contacting a cell or population of cells, or an extract thereof, with a binding agent capable of binding specifically to a target kinase of Table 1. The cell or population of cells will be known or suspected to be or to comprise cells affected by the disorder.

The binding agent may bind to either the target kinase protein or to RNA (e.g. mRNA or precursor mRNA) encoding the target kinase. Thus, in this context and throughout this specification, the binding agent is capable of binding to an expression product, either protein or RNA, of the gene encoding the target kinase.

Also provided is a method for identifying a kinase which is abnormally expressed (upregulated/overexpressed or downregulated/underexpressed) in a proliferative disorder, comprising contacting a cell or population of cells affected by the disorder with a plurality of binding agents each capable of binding specifically and independently to a kinase, wherein at least one of said kinases is a target kinase of Table 1.

The method may comprise contacting the cell or cells with binding agents capable of binding specifically and independently to a plurality of kinases of Table 1, e.g. to at least 2, 5, 10, 15, 20, 25, 30, 40, 50, 60, 70 or to substantially all of the target kinases of Table 1. Binding agents for specific other kinases may also be employed, e.g. for kinases already known to be involved in the cell cycle. Thus, for example, the method may employ binding agents specific for any or all of the kinases of Table 2.

These methods may be performed in vivo or in vitro. However it is likely that the target kinase for which the binding agent is specific will be localised intracellularly, so in preferred embodiments the method is performed in vitro using a cell or population of cells obtained from a subject suspected of suffering from a proliferative disorder. Where whole cells are used, rather than cell extracts, the cells may be permeabilised to allow the binding agent to cross the plasma membrane. Alternatively small and/or hydrophobic binding agents capable of traversing the membrane may be used.

The methods may comprise comparing the presence, absence or degree of binding with that found in the same or similar tissues of healthy subjects and/or subjects known to be affected by the disorder. Thus the method may comprise comparing the results obtained from the test subject with results obtained with a cell or population of cells from one or more subjects known not to suffer from the disorder, i.e. a normal control, and/or one or more subjects known to be affected by the disorder.

The method may further comprise the step of obtaining a cell or population of cells, e.g. a tissue sample or biopsy, from the subject.

Abnormal expression of a kinase in cells from a patient, as compared to normal controls, is indicative of abnormal proliferation of those cells. It may also suggest that the kinase may be a therapeutic target for treatment of the condition. Thus, having identified a particular kinase as being abnormally regulated in a particular disorder, the patient may be treated with a modulator of expression or activity of that kinase.

The target kinases of Table 1, when inhibited, tend to increase the proportion of cells stalled or blocked at some stage of the cell cycle.

Thus a modulator which inhibits activity or expression of the target kinases may be suitable for the inhibition of cell proliferation. A modulator which up-regulates activity or expression of these kinases may also have therapeutic potential. Such modulators may be referred to as target kinase inhibitors and activators respectively.

Modulators, particularly those which inhibit activity or expression of any of the target kinases of the invention in a given cell may induce apoptosis of that cell.

The kinases may themselves be useful agents, e.g. for gene therapy. This may be particularly the case in proliferating cells which carry mutations in the gene for that particular kinase. Introduction of such a kinase may also induce apoptosis in a proliferating cell.

The present invention therefore provides a vector, comprising a coding sequence for a target kinase of the present invention operably linked to suitable transcriptional regulatory sequences. The invention further provides such a vector for use in a method of gene therapy, e.g. for proliferative disease.

The target kinases of the invention act at various stages of the cell cycle including G1, G2, S or M phase. Particularly important target kinases may act at the transition points between these phases. Within M phase a target kinase may act during prophase, prometaphase, metaphase, anaphase or telophase, or at the transition points between these phases. In this regard, the skilled person is referred here to Table 2, which provides a summary of the phenotypes obtained on inhibition of each of these kinases.

Inhibition of each target kinase produces one or more of a number of phenotypes, including a change in mitotic index of the cell population, defects in number or position of centrosomes, defects in number, position or morphology of the spindle, and defects in number, alignment condensation or segregation of the chromosomes.

### Modulators of Kinase Activity or Expression

Modulators of target kinase activity or expression include substances capable of binding to and either stimulating or inhibiting (preferably inhibiting) activity of the kinase protein, i.e. kinase activators or inhibitors. Inhibitors may be competitive inhibitors, capable of interfering with binding of ATP or substrate to the molecule, or may act in an allosteric fashion, binding to a different site on the molecule.

Preferably they are specific for the particular target kinase, that is to say they bind to and inhibit that kinase in preference to others under physiological conditions. The Ki of the inhibitor for the target kinase is preferably at least 2 fold, preferably at least 10 fold, more preferably at least 100 or 1000 fold greater than for other kinase molecules.

The modulator may be a protein or polypeptide of 50 amino acids in size or greater, or a peptide of up to 50 amino acids in length. Typically a peptide will be from 5 to 50 amino acids in length, more typically 10 to 20 amino acids in length. Alternatively the binding agent may be a small molecule e.g. of 1000 Da or less, preferably 750 Da or less, preferably 500 Da or less.

The activity of a target kinase can be measured by following phosphorylation of a substrate molecule. This involves the transfer of a phosphate group from a donor molecule, typically ATP, to the substrate which is typically a protein or peptide containing a serine, threonine or tyrosine residue as an acceptor for the phosphate group. The skilled person is aware of numerous suitable protocols for assaying kinase activity and will be capable of designing a suitable protocol for use in any particular instance. Typically the assay will use ATP having a detectable gamma-phosphate group as a donor molecule. For example, the gamma phosphate group may be radiolabelled. The kinase may be present in a cell extract or may be purified or partly purified from a cell. Alternatively, the assay may be performed in whole cells. Such assays may be qualitative or quantitative.

Modulators of target kinase activity may be further modified to increase their suitability for in vivo administration.

By contrast, modulators of target kinase expression will typically be nucleic acid molecules capable of hybridising to genomic DNA, mRNA or precursor mRNA encoding the kinase. They may be single stranded or double stranded. Such modulators include anti-sense RNA or DNA, triple helix-forming molecules, RNAi, siRNA and ribozymes.

Antisense RNA and DNA molecules act to directly block the translation of mRNA by hybridising to targeted mRNA and preventing protein translation. With respect to antisense DNA, oligodeoxy-ribonucleotides derived from the translation initiation site, e.g. between the −10 and +10 regions of the target gene nucleotide sequence of interest, are preferred.

In using anti-sense genes or partial gene sequences to down-regulate gene expression, a nucleotide sequence is placed under the control of a promoter in a “reverse orientation” such that transcription yields RNA which is complementary to normal mRNA transcribed from the “sense” strand of the target gene. See, for example, Rothstein et al, 1987; Smith et al, (1988) Nature 334, 724-726; Zhang et al, (1992) The Plant Cell 4, 1575-1588, English et al., (1996) The Plant Cell 8, 179-188. Antisense technology is also reviewed in Bourque, (1995), Plant Science 105, 125-149, and Flavell, (1994) PNAS USA 91, 3490-3496.

The complete sequence corresponding to the coding sequence need not be used. For example fragments of sufficient length may be used. It is a routine matter for the person skilled in the art to screen fragments of various sizes and from various parts of the coding sequence to optimise the level of anti-sense inhibition. It may be advantageous to include the initiating methionine ATG codon, and perhaps one or more nucleotides upstream of the initiating codon. A further possibility is to target a conserved sequence of a gene, e.g. a sequence that is characteristic of one or more genes, such as a regulatory sequence.

The sequence employed may be 500 nucleotides or less, possibly about 400 nucleotides, about 300 nucleotides, about 200 nucleotides, or about 100 nucleotides. It may be possible to use oligonucleotides of much shorter lengths, 14-23 nucleotides, although longer fragments, and generally even longer than 500 nucleotides are preferable where possible.

It may be preferable that there is complete sequence identity in the sequence used for down-regulation of expression of a target sequence, and the target sequence, though total complementarity or similarity of sequence is not essential. One or more nucleotides may differ in the sequence used from the target gene. Thus, a sequence employed in a down-regulation of gene expression in accordance with the present invention may be a wild-type sequence (e.g. gene) selected from those available, or a mutant, derivative, variant or allele, by way of insertion, addition, deletion or substitution of one or more nucleotides, of such a sequence. The sequence need not include an open reading frame or specify an RNA that would be translatable. It may be preferred for there to be sufficient homology for the respective anti-sense and sense RNA molecules to hybridise. There may be down regulation of gene expression even where there is about 5%, 10%, 15% or 20% or more mismatch between the sequence used and the target gene.

Double stranded RNA (dsRNA) has been found to be even more effective in gene silencing than antisense strands alone (Fire A. et al Nature, Vol 391, (1998)). dsRNA mediated silencing is gene specific and is often termed RNA interference (RNAi).

RNA interference is a two step process. First, dsRNA is cleaved within the cell to yield short interfering RNAs (siRNAs) of about 21-23 nt length with 5′ terminal phosphate and 3′ short overhangs (˜2 nt) The siRNAs target the corresponding mRNA sequence specifically for destruction (Zamore P. D. Nature Structural Biology, 8, 9, 746-750, (2001)

RNAi may be also be efficiently induced using chemically synthesized siRNA duplexes of the same structure with 3′-overhang ends (Zamore P D et al Cell, 101, 25-33, (2000)). Synthetic siRNA duplexes have been shown to specifically suppress expression of endogenous and heterologeous genes in a wide range of mammalian cell lines (Elbashir S M. et al. Nature, 411, 494-498, (2001)).

See also Fire (1999) Trends Genet. 15: 358-363, Sharp (2001) Genes Dev. 15: 485-490, Hammond et al. (2001) Nature Rev. Genes 2: 1110-1119 and Tuschl (2001) Chem. Biochem. 2: 239-245.

Ribozymes are enzymatic RNA molecules capable of catalysing the specific cleavage of RNA. (For a review, see Rossi, J., 1994, Current Biology 4: 469-471). The mechanism of ribozyme action involves sequence specific hybridisation of the ribozyme molecule to complementary target RNA, followed by an endonucleolytic cleavage. The composition of ribozyme molecules must include one or more sequences complementary to the target protein mRNA, and must include the well known catalytic sequence responsible for mRNA cleavage. For this sequence, see U.S. Pat. No. 5,093,246, which is incorporated by reference herein in its entirety. As such, within the scope of the invention are engineered hammerhead motif ribozyme molecules that specifically and efficiently catalyse endonucleolytic cleavage of RNA sequences encoding target proteins.

Specific ribozyme cleavage sites within any potential RNA target are initially identified by scanning the molecule of interest for ribozyme cleavage sites which include the following sequences, GUA, GUU and GUC. Once identified, short TNA sequences of between 15 and 20 ribonucleotides corresponding to the region of the target protein gene, containing the cleavage site may be evaluated for predicted structural features, such as secondary structure, that may render the oligonucleotide sequence unsuitable. The suitability of candidate sequences may also be evaluated by testing their accessibility to hybridise with complementary oligonucleotides, using ribonuclease protection assays.

Nucleic acid molecules to be used in triplex helix formation for the inhibition of transcription should be single stranded and composed of deoxynucleotides. The base composition of these oligonucleotides must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC⁺ triplets across the three associated strands of the resulting triple helix. The pyrimidine-rich molecules provide base complementary to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so-called “switchback” nucleic acid molecule. Switchback molecules are synthesised in an alternating 5′-3′, 3′-5′ manner, such that they base pair with first one strand of a duplex and then the other, eliminating the necessity for a sizeable stretch of either purines or pyrimidines to be present on one strand of a duplex.

Table 1 shows accession numbers for amino acid sequences of the target kinases shown in that table. From this information, the skilled person will be able to obtain the corresponding nucleotide sequences, and from there design appropriate nucleic acid modulators.

### Binding Agents

A target kinase and a binding agent specific for that kinase preferably form a specific binding pair. The term “specific binding pair” may be used to describe a pair of molecules comprising a specific binding member (sbm) and a binding partner (bp) therefor which have particular specificity for each other and which in normal conditions bind to each other in preference to binding to other molecules. Examples of specific binding pairs are antigens and antibodies, ligands (such as hormones, etc.) and receptors, avidin/streptavidin and biotin, lectins and carbohydrates, and complementary nucleotide sequences.

Preferably the interaction between the target kinase and the binding agent is a specific interaction. By “specific” is meant that the particular binding sites of the binding agent will not show any significant binding to other molecules (e.g. other molecules in the assay). Preferably the interaction between the binding agent and the target kinase has a K_{D} of the order of 10⁻⁶ to 10⁻⁹M or smaller. In any particular assay the affinity of the binding agent for the target kinase is preferably at least 10 fold greater than for other molecules in the assay, preferably greater than 20 fold, preferably greater than 50 fold, and more preferably greater than 100 fold.

The binding agent may bind to any suitable portion of the target kinase including the substrate binding site. The binding agent may be a protein or polypeptide of 50 amino acids in size or greater, or a peptide of up to 50 amino acids in length. Typically a peptide will be from 5 to 50 amino acids in length, more typically 10 to 20 amino acids in length. Alternatively the binding agent may be a small molecule e.g. of 1000 Da or less, preferably 750 Da or less, preferably 500 Da or less.

Antibodies are preferred examples of binding agents. Thus preferred assay formats for diagnosis are immunological assays including ELISA assays, and immunohistochemistry, which may be carried out on whole cells or tissue sections, other forms of immunostaining for FACS analysis, confocal microscopy or the like, which may be carried out on single cells or populations of dispersed cells, and immunoblotting, which is suitable for analysis of cell extracts.

It has been shown that fragments of a whole antibody can perform the function of binding antigens. The term “antibody” is therefore used herein to encompass any molecule comprising the binding fragment of an antibody. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341, 544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding member (Bird et al, Science, 242, 423-426, 1988; Huston et al, PNAS USA, 85, 5879-5883, 1988).

Methods for determining the concentration of analytes in samples from individuals are well known in the art and readily adapted by the skilled person in the context of the present invention to determine the presence or amount of the kinase or fragments thereof. Thus the binding agents described herein may be used in diagnostic methods which may allow a physician to determine whether a patient suffers from or is at risk of developing a proliferative disorder. It may also allow the physician to optimise the treatment of the disorder. Thus, this allows for planning of appropriate therapeutic and/or prophylactic treatment, permitting stream-lining of treatment by targeting those most likely to benefit.

The methods typically employ a biological sample from patient such as blood, serum, tissue, serum, urine or other suitable body fluids.

Assay methods for determining the concentration of protein markers typically employ binding agents having binding sites capable of specifically binding to protein markers, or fragments thereof, or antibodies in preference to other molecules. Examples of binding agents include antibodies, receptors and other molecules capable of specifically binding the analyte of interest. Conveniently, the binding agents are immobilised on solid support, e.g. at defined, spatially separated locations, to make them easy to manipulate during the assay.

The sample is generally contacted with the binding agent(s) under appropriate conditions which allow the analyte in the sample to bind to the binding agent(s). The fractional occupancy of the binding sites of the binding agent(s) can then be determined either by directly or indirectly labelling the analyte or by using a developing agent or agents to arrive at an indication of the presence or amount of the analyte in the sample. Typically, the developing agents are directly or indirectly labelled (e.g. with radioactive, fluorescent or enzyme labels, such as horseradish peroxidase) so that they can be detected using techniques well known in the art. Directly labelled developing agents have a label associated with or coupled to the agent. Indirectly labelled developing agents may be capable of binding to a labelled species (e.g. a labelled antibody capable of binding to the developing agent) or may act on a further species to produce a detectable result. Thus, radioactive labels can be detected using a scintillation counter or other radiation counting device, fluorescent labels using a laser and confocal microscope, and enzyme labels by the action of an enzyme label on a substrate, typically to produce a colour change. In further embodiments, the developing agent or analyte is tagged to allow its detection, e.g. linked to a nucleotide sequence which can be amplified in a PCR reaction to detect the analyte. Other labels are known to those skilled in the art are discussed below. The developing agent(s) can be used in a competitive method in which the developing agent competes with the analyte for occupied binding sites of the binding agent, or non-competitive method, in which the labelled developing agent binds analyte bound by the binding agent or to occupied binding sites. Both methods provide an indication of the number of the binding sites occupied by the analyte, and hence the concentration of the analyte in the sample, e.g. by comparison with standards obtained using samples containing known concentrations of the analyte.

In alternative embodiments, the analyte can be tagged before applying it to the support comprising the binding agent.

Preferred formats are ELISA assays and immunostaining (e.g. immunohistochemistry).

There is also an increasing tendency in the diagnostic field towards miniaturisation of such assays, e.g. making use of binding agents (such as antibodies or nucleic acid sequences) immobilised in small, discrete locations (microspots) and/or as arrays on solid supports or on diagnostic chips. These approaches can be particularly valuable as they can provide great sensitivity (particularly through the use of fluorescent labelled reagents), require only very small amounts of biological sample from individuals being tested and allow a variety of separate assays to be carried out simultaneously. This latter advantage can be useful as it provides an assay employing a plurality of analytes to be carried out using a single sample. Examples of techniques enabling this miniaturised technology are provided in WO84/01031, WO88/1058, WO89/01157, WO93/8472, WO95/18376/ WO95/18377, WO95/24649 and EP 0 373 203 A. Thus, in a further aspect, the present invention provides a kit comprising a support or diagnostic chip having immobilised thereon a plurality of binding agents capable of specifically binding different protein markers or antibodies, optionally in combination with other reagents (such as labelled developing reagents) needed to carrying out an assay. In this connection, the support may include binding agents specific for analytes such as vimentin, e.g. as disclosed in U.S. Pat. No. 5,716,787.

Alternatively the binding agent may also be a nucleic acid molecule capable of binding to mRNA or precursor mRNA. Thus mRNA or precursor mRNA encoding the target kinase may be detected by hybridisation with a probe having a suitable complementary sequence, e.g. by Northern blotting or in situ hybridisation. Such protocols may use probes of at least about 20-80 bases in length. The probes may be of 100, 200, 300, 400 or 500 bases in length or more. Binding assays may be conducted using standard procedures, such as described in Sambrook et al., Molecular Cloning A Laboratory Manual (New York: Cold Spring Harbor Laboratory Press, 1989 or later editions).

Alternatively, conventional RT PCR procedures (including quantitative PCR procedures) may be used to analyse the presence or amount of mRNA or precursor mRNA in a given sample. A suitable primer having at least 15 to 20 bases complementary to the target kinase mRNA or precursor mRNA sequence will typically be used to prime cDNA synthesis. Subsequently, a segment of the cDNA is amplified in a PCR reaction using a pair of nucleic acid primers. The skilled person will be able to design suitable probes or primers based on the publicly available sequence data for the target kinases of Table 1.

Whether it is a protein, peptide, small molecule or nucleic acid, the binding agent may also act as an activator or inhibitor of the kinase expression or activity.

### Pharmaceutical Compositions

The modulators of the invention can be formulated in pharmaceutical compositions. These compositions may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Whether it is a polypeptide, antibody, peptide, nucleic acid molecule, small molecule or other pharmaceutically useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Suitable carriers, adjuvants, excipients, etc. can be found in standard pharmaceutical texts, for example Remington's Pharmaceutical Sciences, 20th Edition, 2000, pub. Lippincott, Williams & Wilkins; and Handbook of Pharmaceutical Excipients, 2nd edition, 1994.

Alternatively, targeting therapies may be used to deliver the active agent more specifically to certain types of cell, by the use of targeting systems such as antibody or cell specific ligands. Targeting may be desirable for a variety of reasons; for example if the agent is unacceptably toxic, or if it would otherwise require too high a dosage, or if it would not otherwise be able to enter the target cells.

Instead of administering these agents directly, they could be produced in the target cells by expression from an encoding gene introduced into the cells, eg in a viral vector (a variant of the VDEPT technique—see below). The vector could be targeted to the specific cells to be treated, or it could contain regulatory elements which are switched on more or less selectively by the target cells.

Alternatively, the agent could be administered in a precursor form, for conversion to the active form by an activating agent produced in, or targeted to, the cells to be treated. This type of approach is sometimes known as ADEPT or VDEPT; the former involving targeting the activating agent to the cells by conjugation to a cell-specific antibody, while the latter involves producing the activating agent, e.g. an enzyme, in a vector by expression from encoding DNA in a viral vector (see for example, EP-A-415731 and WO 90/07936).

A composition may be administered alone or in combination with other treatments, either simultaneously or sequentially dependent upon the condition to be treated.

### Gene Therapy

Nucleic acids encoding modulators of target kinase expression (e.g. antisense, RNAi, siRNA or ribozyme molecules) may be used in methods of gene therapy (as may the kinases themselves). A construct capable of expressing such nucleic acid may be introduced into cells of a recipient by any suitable means, such that the relevant sequence is expressed in the cells.

The construct may be introduced in the form of naked DNA, which is taken up by some cells of animal subjects, including muscle cells of mammalians. In this aspect of the invention the construct will generally be carried by a pharmaceutically acceptable carrier alone. The construct may also formulated in a liposome particle, as described above.

Such methods of gene therapy further include the use of recombinant viral vectors such as adenoviral or retroviral vectors which comprise a construct capable of expressing a polypeptide of the invention. Such viral vectors may be delivered to the body in the form of packaged viral particles.

Constructs of the invention, however formulated and delivered, will be for use in treating tumours in conjunction with therapy. The construct will comprise the relevant nucleic acid linked to a promoter capable of expressing it in the target cells. The constructs may be introduced into cells of a human or non-human mammalian recipient either in situ or ex-vivo and reimplanted into the body. Where delivered in situ, this may be by for example injection into target tissue(s) or in the case of liposomes, inhalation.

Gene therapy methods are widely documented in the art and may be adapted for use in the expression of the required sequence.

Although the invention has been described above primarily with reference to the kinases (“target” kinases) of Table 1, it will readily be understood that the methods of the invention may be applied equally well to any of the kinases in Table 2. References to kinases of Table 1 should be construed accordingly.

TABLE 1

| | | Orthologous human | |
|---|---|---|---|
| Drosophila gene | Accession | gene | Accession |
| gek | Q9W1B0 | CDC42-BINDING | Q9Y5S2; Q9H521 |
| | | PROTEIN KINASE BETA | |
| | | CDC42 binding | O75039 |
| | | protein kinase alpha | |
| | | (DMPK-like) | |
| gwl CG7719 | Q95TN8 | KIAA0807 protein | EMBL: AB018350 |
| | | FLJ14813 | protein_id: BAA34527 |
| | | MASTL | GenBank gi: 3882335 |
| Ilk | Q9NHC7, | INTEGRIN-LINKED | ILK1 (Q13418) |
| | Q9V400 | PROTEIN KINASE 1; | ILK2 (P57043) |
| | | INTEGRIN-LINKED | |
| | | PROTEIN KINASE 2 | |
| Mkk4 | O61444 | DUAL SPECIFICITY | P45985 |
| | | MITOGEN-ACTIVATED | |
| | | PROTEIN KINASE | |
| | | KINASE 4 | |
| tkv | Q27933 | BONE MORPHOGENETIC | BMPR1B (O00238); |
| | Q24326 | PROTEIN RECEPTOR | BMPR1A (P36894); |
| | | TYPE IB PRECURSOR; | P78366 |
| | | BONE MORPHOGENETIC | |
| | | PROTEIN RECEPTOR | |
| | | TYPE IA PRECURSOR | |
| dnt | Q9V422 | TYROSINE-PROTEIN | P34925; Q04696 |
| | | KINASE RYK PRECURSOR | RYK_HUMAN |
| Nrk | Q9V6K3 | MUSCLE SPECIFIC | O15146 |
| | | TYROSINE KINASE | |
| | | RECEPTOR | |
| Gcn2 | Q9V9X8 | KIAA1338 PROTEIN | Q9P2K8 |
| | | (FRAGMENT) | |
| | | (EIF2AK4) | |
| | | SERINE/THREONINE | O00506; Q15522 |
| | | PROTEIN KINASE 25 | |
| CG10967/ | Q8MQJ7 | Similar to unc-51- | Q8IYT8 (O75119) |
| l(3)00305 | | like kinase 2; | ULK1_HUMAN (O75385) |
| | | KIAA0623 PROTEIN; | |
| | | Serine/threonine- | |
| | | protein kinase ULK1 | |
| SNF4Agamma | O96613 | 5′-AMP-ACTIVATED | Q9UGJ0; Q9NUZ9; |
| | | PROTEIN KINASE, | Q9UDN8; Q9ULX8 |
| | | GAMMA-2 SUBUNIT | |
| | | (AMPK GAMMA-2 CHAIN) | |
| | | (AMPK GAMMA2) | |
| Pvr | Q95P10 | VASCULAR ENDOTHELIAL | P17948 (VEGFR-1); |
| | | GROWTH FACTOR | O60722; P16057; |
| | | RECEPTOR 1 PRECURSOR | Q12954 |
| | | VASCULAR ENDOTHELIAL | P35968 (VEGFR-2); |
| | | GROWTH FACTOR | |
| | | RECEPTOR 2 | |
| | | PRECURSOR; | |
| | | VASCULAR ENDOTHELIAL | P35916 (VEGFR-3) |
| | | GROWTH FACTOR | |
| | | RECEPTOR 3 PRECURSOR | |
| CG7236 | Q9VMN3 | SERINE/THREONINE- | Q00532 (KKIA_HUMAN) |
| | | PROTEIN KINASE | |
| | | KKIALRE (EC 2.7.1.—) | |
| | | (CYCLIN-DEPENDENT | |
| | | KINASE-LIKE 1) | |
| CG2829/BcDNA:GH07910 | Q9W4Q4 | KIAA0137 PROTEIN | Q86UE8; Q9UKI7; |
| | | (FRAGMENT) | Q9Y4F7; Q9UKI8; |
| | | TOUSLED-LIKE KINASE 2 | Q14150; Q8N591; |
| | | | Q9NYH2; Q9Y4F6 |
| | | PKU-BETA; TOUSLED- | Q9UKI8; Q14150; |
| | | LIKE KINASE 1 | Q8N591; Q9NYH2; |
| | | | Q9Y4F6 |
| Lkb1/CG9374 | Q9VFS7 | SERINE/THREONINE- | GenBank gi: |
| | | PROTEIN KINASE 11 | 4507271; Q15831 |
| | | (EC 2.7.1.—) | |
| | | (SERINE/THREONINE- | |
| | | PROTEIN KINASE LKB1) | |
| Par-1 | Q9V8V8; | CDC25C ASSOCIATED | P27448; O60219; |
| | Q963E6 | PROTEIN KINASE C- | Q8TB41; Q8WX83; |
| | | TAK1 | Q96RG1; Q9UMY9; |
| | | | Q9UN34 |
| for2 | P32023; | CGMP-DEPENDENT | Q13976 |
| | Q9VQT2 | PROTEIN KINASE 1, | (KGPA_HUMAN); |
| | | ALPHA ISOZYME; | P14619 (KGPB_HUMAN) |
| | | CGMP-DEPENDENT | |
| | | PROTEIN KINASE 1, | |
| | | BETA ISOZYME E | |
| htl | Q07407 | FIBROBLAST GROWTH | P21802; P18443; |
| | | FACTOR RECEPTOR 2 | Q01742; Q12922; |
| | | PRECURSOR (EC | Q14300; Q14301; |
| | | 2.7.1.112) (FGFR-2) | Q14302; Q14303; |
| | | (KERATINOCYTE GROWTH | Q14304; Q14305; |
| | | FACTOR RECEPTOR) | Q14672; Q14718; |
| | | | Q14719; Q96KL9; |
| | | | Q96KM0; Q96KM1; |
| | | | Q96KM2; Q9NZU2; |
| | | | Q9NZU3; Q9UD01; |
| | | | Q9UD02; Q9UIH3; |
| | | | Q9UIH4; Q9UIH5; |
| | | | Q9UIH6; Q9UIH7; |
| | | | Q9UIH8; Q9UM87; |
| | | | Q9UMC6; Q9UNS7; |
| | | | Q9UQH7; Q9UQH8; |
| | | | Q9UQH9; Q9UQI0 |
| | | FIBROBLAST GROWTH | P22607; Q14308; |
| | | FACTOR RECEPTOR 3 | Q16294; |
| | | PRECURSOR; | Q16608(FGFR3) |
| | | FIBROBLAST GROWTH | P11362 (FGFR1); |
| | | FACTOR RECEPTOR 1; | P22455 (FGFR4) |
| | | FIBROBLAST GROWTH | |
| | | FACTOR RECEPTOR 4 | |
| CG8565 | Q9VXN5 | SERINE/THREONINE | Q8IYQ3; O00311; |
| | | KINASE 23 | O00558 |
| | | EC_2.7.1.37 MUSCLE | |
| | | SPECIFIC SERINE | |
| | | KINASE 1 MSSK 1; | |
| | | CELL DIVISION CYCLE | |
| | | 7-RELATED PROTEIN | |
| | | KINASE (EC 2.7.1.—) | |
| | | (CDC7-RELATED | |
| | | KINASE) (HSCDC7) | |
| | | (HUCDC7) | |
| drl | Q27324 | TYROSINE-PROTEIN | P34925; Q04696; |
| | | KINASE RYK PRECURSOR | RYK_HUMAN |
| fray | Q9VE62 | OXIDATIVE-STRESS | O95747 |
| | | RESPONSIVE 1 | |
| CG15072 | Q9V8L2 | KIAA0999 PROTEIN | Q9Y2K2 |
| | | (FRAGMENT) | |
| lic | O62602 | DUAL SPECIFICITY | P46734; Q99441; |
| | | MITOGEN-ACTIVATED | Q9UE71; Q9UE72 |
| | | PROTEIN KINASE | |
| | | KINASE 3 (EC 2.7.1.—) | |
| | | (MAP KINASE KINASE | |
| | | 3) (MAPKK 3) | |
| | | (MAPK/ERK KINASE 3) | |
| | | DUAL SPECIFICITY | P52564 |
| | | MITOGEN-ACTIVATED | |
| | | PROTEIN KINASE | |
| | | KINASE 6 | |
| | | MAP KINASE 3B | P46734; Q99441; |
| | | | Q9UE71; Q9UE72 |
| | | MAP KINASE 3C | P46734; Q99441; |
| | | | Q9UE71; Q9UE72 |
| SAK | O97143 | SAK | O00444 |
| | | SERINE/THREONINE | |
| | | PROTEIN KINASE | |
| | | (PLK4) | |
| Hippo/CG11228 | Q9V8W4 | SERINE/THREONINE | Q8NBU1; Q13188; |
| | | PROTEIN KINASE 3 (EC | Q15445; Q15801 |
| | | 2.7.1.37) (STE20- | |
| | | LIKE KINASE MST2) | |
| | | (MST-2) (MAMMALIAN | |
| | | STE20-LIKE PROTEIN | |
| | | KINASE 2) | |
| | | (SERINE/THREONINE | |
| | | PROTEIN KINASE KRS- | |
| | | 1) | |
| | | SERINE/THREONINE | Q13043 |
| | | PROTEIN KINASE 4 | |
| Pka-C2 | Q9VA46 | CAMP-DEPENDENT | P22694; Q8IYR5 |
| | | PROTEIN KINASE, | |
| | | BETA-CATALYTIC | |
| | | SUBUNIT | |
| CG7643/Mps1 | Q9VEH1 | DUAL SPECIFICITY | Q9BW51; P33981 |
| | | PROTEIN KINASE TTK | |
| | | (EC 2.7.1.—) (PYT) | |
| nmo | Q8IQ91 | NEMO-LIKE KINASE | Q9UBE8 |
| trbl | Q9V3Z1 | GS3955 (GS3955 | Q92519 |
| | | PROTEIN) TRB2 | |
| | | TRB1 | Q9H2Y8 |
| | | TRB3 | Q96RU7 |
| gish | Q8INB6 | CASEIN KINASE I, | Q9Y6M4; Q9Y6M3 |
| | | GAMMA 3 ISOFORM (EC | |
| | | 2.7.1.—) (CKI-GAMMA | |
| | | 3) | |
| | | CASEIN KINASE I, | P78368 |
| | | GAMMA 2 ISOFORM | |
| | | CASEIN KINASE I, | Q9HCP0 |
| | | GAMMA 1 ISOFORM | |
| CkIIalpha | P08181 | CASEIN KINASE II, | KC22_HUMAN |
| | | ALPHA CHAIN (CK II) | P19138; P20426; |
| | | | Q14013 |
| ik2 | Q8INU8 | TANK BINDING KINASE | Q9UHD2 |
| | | TBK1 (NF-KB- | |
| | | ACTIVATING KINASE | |
| | | NAK) | |
| | | Inhibitor at nuclear | Q14164 |
| | | factor kappa-B | |
| | | kinase epsilon | |
| | | subunit (EC 2.7.1.—) | |
| | | (I kappa-B kinase | |
| | | epsilon) (IkBKE) | |
| | | (IKK-epsilon) (IKK- | |
| | | E) (Inducible I | |
| | | kappa-B kinase) | |
| | | (IKK-i). | |
| mnb | P49657 | DUAL-SPECIFICITY | Q13627; O60769; |
| | | TYROSINE- | Q92582; Q92810; |
| | | PHOSPHORYLATION | Q9UNM5 |
| | | REGULATED KINASE 1A | |
| | | (EC 2.7.1.—) | |
| | | (PROTEIN KINASE | |
| | | MINIBRAIN HOMOLOG) | |
| | | (MNBH) (HP86) (DUAL | |
| | | SPECIFICITY YAK1- | |
| | | RELATED KINASE) | |
| SNF1A | O18645 | AMP-ACTIVATED | P54646; Q9H1E8; |
| | | PROTEIN KINASE, | Q9UD43 |
| | | AMPK, catalytic | |
| | | alpha-2 chain | |
| | | PRKAA1 protein | Q86VS1 |
| MAPk-Ak2 | P49071 | MAP KINASE-ACTIVATED | P49137 |
| | | PROTEIN KINASE 2 (EC | |
| | | 2.7.1.—) (MAPK- | |
| | | ACTIVATED PROTEIN | |
| | | KINASE 2) (MAPKAP | |
| | | KINASE 2) (MAPKAPK- | |
| | | 2) | |
| | | Mitogen activated | Q16644 |
| | | protein kinase | |
| | | activated protein | |
| | | kinase-3 | |
| CG7156 | Q9VEA9 | RSK-like protein, | Q8TDD3 |
| | | HYPOTHETICAL 60.0 KDA | Q9Y6S9 |
| | | PROTEIN | |
| CG1951 | Q9VAR0 | KIAA1360 PROTEIN | Q9P2I7 |
| | | (FRAGMENT) | Q9NWE9 |
| | | Hypothetical protein | Q96ST4 |
| | | FLJ14645 | Q9H7V5 |
| | | Hypothetical protein | Q96EF4 |
| | | FLJ14212 | Q9NVH3 |
| | | Hypothetical protein | |
| | | (Fragment) | |
| | | Hypothetical protein | |
| | | FLJ10735 | |
| CkIIbeta | P08182 | CASEIN KINASE II | P13862; P07312 |
| | | BETA CHAIN (CK II) | |
| | | (PHOSVITIN) (G5A | |
| CkIalpha | P54367 | CASEIN KINASE I, | P48729; Q96HD2 |
| | | ALPHA ISOFORM (EC | KC1A_HUMAN; Q8WXF2 |
| | | 2.7.1.—) (CKI-ALPHA) | |
| | | (CK1) | |
| CG18582/mbt | Q9VXE5 | SERINE/THREONINE- | GenBank gi: 4101586 |
| | | PROTEIN KINASE PAK 5 | |
| | | (EC 2.7.1.—) (P21- | |
| | | ACTIVATED KINASE 5) | |
| | | (PAK-5) | |
| | | (P21-ACTIVATED | Q9P286 |
| | | KINASE 7) (PAK-7) | |
| | | (P21-ACTIVATED | O96013 |
| | | KINASE 4) (PAK-4) | |
| CG6498/MAST | Q8MSY6 | MAST1; KIAA0973 | Q9Y2H9 |
| | | PROTEIN (FRAGMENT) | |
| CG1344 | Q9Y0Z6 | EZRIN BINDING | GenBank gi: |
| | | PROTEIN PACE-1 | 27363466 |
| CG2309 | Q9W354 | Extracellular | Q8TD08 |
| | | signal-regulated | Q8N362 |
| | | kinase 8 | |
| CG9488/Ddr | Q9VMF6 | Discoidin domain | DDR2_HUMAN; Q16832 |
| | | receptor 2 | |
| Eip63E | Q8IRC9 | Serine/threonine | PFT1_HUMAN; O94921 |
| | | protein kinase | |
| | | PFTAIRE-1 | |
| Doa | P49762 | Dual specificity | CLK2_HUMAN; P49760 |
| | | protein kinase CLK2 | |
| | | (EC 2.7.1.37) (EC | |
| | | 2.7.1.112) (CDC like | |
| | | kinase 2) | |
| | | Dual specificity | CLK3_HUMAN; P49761 |
| | | protein kinase CLK3 | |
| | | (EC 2.7.1.37) (EC | |
| | | 2.7.1.112) (CDC like | |
| | | kinase 3) | |
| | | Dual specificity | CLK4_HUMAN; Q9HAZ1 |
| | | protein kinase CLK4 | |
| | | (EC 2.7.1.37) (EC | |
| | | 2.7.1.112) (CDC like | |
| | | kinase 1) | |
| CG3216 | Q8MLX0 | Atrial natriuretic | ANPA_HUMAN; P16066 |
| | | peptide receptor A | |
| | | precursor (ANP-A) | |
| | | (ANPRA) (GC-A) | |
| | | (Guanylate cyclase) | |
| | | (EC 4.6.1.2) (NPR-A) | |
| | | Atrial natriuretic | ANPB_HUMAN; P20594 |
| | | peptide receptor B | |
| | | precursor (ANP-B) | |
| | | (ANPRB) (GC-B) | |
| | | (Guanylate cyclase) | |
| | | (EC 4.6.1.2) (NPR-B) | |
| | | (Atrial natriuretic | |
| | | peptide B-type | |
| | | receptor) | |
| CG5483 | Q9VDJ9 | LRRK1 (Hypothetical | Q96JN5 |
| | | protein KIAA1790) | |
| CG7597 | Q8T9E1 | CDC2L5 protein | CDL5_HUMAN |
| | | kinase | Q9H4A0; Q14004 |
| | Q9VP22 | Cell division cycle | CRK7_HUMAN; Q9NYV4 |
| | | 2-related protein | |
| | | kinase 7 (EC 2.7.1.—) | |
| | | (CDC2-related | |
| | | protein kinase 7) | |
| | | (CrkRS) | |
| Fs(1)h | P13709 | BRD4/MCAP | Q9ESU6 |
| | | RING3 | P25440 |
| PhKgamma | Q9I7D0 | PhKgamma1 | Q16816 |
| | | PHKG2 | Q16221 |
| PK92B | Q9VDS9 | MITOGEN ACTIVATED | Q13233 |
| | | KINASE KINASE KINASE 1 | |
| pll | Q05652 | IRAK1 | P51617; Q7Z5V4; |
| | | | Q96RL2; Q96C06 |
| | | IRAK2 | O43187 |

Accession numbers are taken from Swiss-Prot Release 42.6 of 28 Nov. 2003; TrEMBL Release 25.6 of 28 Nov. 2003, GenBank Release 138.0 of 20 Oct. 2003, UniProt Release 3.3, and FlyBase (5 Dec. 2004).

The disclosure of all references cited herein, insofar as it may be used by those skilled in the art to carry out the invention, is hereby specifically incorporated herein by cross-reference.

### DESCRIPTION OF THE DRAWINGS

FIG. 1—Screening protocol. a) A protein kinase (PK) data set of 228 protein kinases was defined based on Morrison et al (2000), Manning et al (2002) and FlyBase (Table 3). b) PCR primers specific for each PK were designed with a T7 RNA polymerase overhang (Table 3). PCR fragments were generated (average 500 bp) from either Drosophila genomic DNA or cDNA. These templates were transcribed to generate dsRNA. c) Drosophila S2 cells were transfected as previously described^{11,47}. GFP and polo dsRNAs were used as negative and positive controls. After 72 hours cells were harvested, fixed and stained for FACS analysis (DNA content (FL2; propidium iodide) and cell size (Forward Light Scatter)) (d) and immunocytochemistry (e-f) Mitotic defects were quantitated blindly by fluorescence microscopy and statistically analysed. 1000-3000 cells were scored per slide (comprising at least 60 mitotic cells). Cells were categorised according to phase of mitosis and to centrosome, spindle and DNA morphology (we defined 20 potential mitotic phenotypic abnormalities) and coded to facilitate computer analysis of the data.

FIG. 2—Cell cycle progression following RNAi of protein kinases. Examples show a control FACS profile in black (open curve); cells transfected with dsRNA for GFP) and one RNAi profile representative of a phenotypic class in grey (hatched curve). FSC: Forward Light Scatter profile reflecting cell size. a) RNAi resulting in an increase in the proportion of cells in G1 can be associated with a reduction in cell size (a1); an increase (a2) or no significant change (a3). b) RNAi resulting in an increase of cells with intermediate DNA content: S phase or aneuploid cells. These have been subdivided according to the extent of accumulation of G2 cells (b1 vs b2). c) RNAi resulting in an increase of cells in G2/M phase could be associated with either an increase in cell size (c1) or not (c2). d) RNAi resulting in an increase in polyploid cells. In all groups, the kinase depicted is indicated under each panel and a list of all enzymes in each category is given within the panel. Names followed by an asterisk indicate kinases for which the RNAi phenotype is weaker.

FIG. 3—Examples of mitotic phenotypes seen following down-regulation of selected protein kinases. a-d) Control cells at a) prophase; b) metaphase; c) late anaphase d) cytokinesis stained to reveal α-tubulin, γ-tubulin and DNA. Lower panels—Selected RNAi phenotypes (name of gene on top left corner) illustrating some scored parameters (lower right hand corner). CNVH—centrosome number very high; CN1—only one pole shows γ-tubulin; CN0—no γ-tubulin at poles; SBR—branched spindle; AS—abnormal spindle; SSP—splayed spindle; CRAD—chromosome alignment defects; CRSD—chromosome segregation defects; CRCD—chromosome condensation defects; CSD—central spindle defects; MC—multiple cytokinesis. Scale bar is 5 μm.

FIG. 4—Quantitative analysis of mitotic RNAi phenotypes. a-c) Ranking of the phenotypic scores (PS; filled squares) for three of the scored categories of mitotic phenotype. PS were obtained after normalisation of each quantitative RNAi parameter in relation to the average of control values for each experiment (Supplementary Material and Methods). Filled circles represent normalised control values (ct). The scored parameters presented are (a) mitotic index (Mi); (b) ratio of cells in prometaphase and metaphase vs total number of mitotic cells (PM); and (c) percentage of spindle abnormalities (SP). Confidence intervals (CI) were defined on the basis of control values (Materials and Methods). The phenotypic score for the majority of kinases fell along a gentle slope that lay within the error limits for the data measurements. At the extremes were cases in which the parameter was either significantly higher or lower than controls (circled). The mitotic parameters were scored in repeat RNAi experiments for all kinases and showed a significant correlation for each of the different variables. d) Kinases showing mitotic phenotypes. Only kinases showing PS values outside of the 90% CI in two independent experiments were considered to have a mitotic phenotype. Individual rows show the phenotype of each kinase. Scored parameters are shown in different columns, the strength of the phenotype is shown in different colours and colour intensity: the extreme arbitrary values −5 and 5 indicate respectively PS values outside the 99% CI at the lower or higher boundary in both experiments; −4 and 4 indicate PS values outside the 95% CI and −3 and 3 indicate PS values outside the 90% CI (see legend in figure). Black indicates PS values within the 90% CI.

FIG. 5—Novel cell cycle roles for Gwl, Fray and PVR kinases. Control cells treated with dsRNA for GFP (a,d,g). RNAi of gwl leads to chromosome segregation and spindle abnormalities. Note the unequal amounts of chromatin at the spindle poles (b,c). In control cells MEI-S332 is lost from centromeres after metaphase (d). After gwl RNAi cells show MEI-S332 staining associated with chromosomes towards the centre of the spindle (e) or at the poles of anaphase-like spindles (f). RNAi of fray leads to severe spindle defects (h, i). j) RNAi of fray and gwl leads to reduction of RNA monitored by RT-PCR. k) RNAi of pvr leads to reduction of protein. l) pvr RNAi leads to an increase in cells with G2 DNA content (rey hatched curve; control cells shown in black, open curve) and the Pvr ligand, pvf2, shows the same phenotype.

FIG. 6—RNAi of regulators gives similar phenotypes to depletion of the kinases. The examples each show a control FACS profile in black (open curve; cells transfected with dsRNA for GFP) and sample profile in grey (hatched curve). a) and b) Depletion of CDK4 gives rise to an increase in the percentage of cells in G1 relative to G2, with a small but consistent increase in cell size. An increase in cell size was also observed after depletion of cyclin D, a regulator of CDK4 activity. c) Depletion of both SNF1a and its regulatory partner SNF4γ gives rise to a consistent increase in the population of cells with S phase DNA content.

FIG. 7—Inhibition of HeLa cell proliferation by RNAi to human orthologues of Drosophila kinases. HeLa cells transfected with 20 nM of diced double stranded RNAi (dsiRNA) towards the identified target kinases, using TransFast reagent (Promega), for 4 hours. i) After 48 h, cells were harvested for RNA using Trizol reagent (Invitrogen). cDNA was synthesized using ‘Cells to cDNA’ (Ambion). This was then used in a QRT-PCR reaction (reagents and protocol from ABI) to quantify amounts of target kinase mRNA in control cells transfected with dsiGFP (white) or those receiving dsiMAST, dsiPLK4, dsiCDC42 BPA, dsiCDC42 BPB, dsiAUKB (Aurora kinase B), or dsiPLK1 (black). ii) Cells transfected with various dsiRNA's were also analysed at 72 h for mitotic index by fixing in 4% formaline, permeabilising in PBS and 0.1% Tx100 (PBST), blocking for 1 h with in PBST and 1% BSA. Cells were incubated overnight at 4 C with an anti-phospho-histone H3 primary antibody (Upstate 06-570) at 1:500 and a secondary antibody (Rhodamine anti-rabbit) at 1:200 for 1 h at RT, whilst washing in between with PBST. Finally cells were incubated with DAPI in PBS for 30 min and washed again prior to analysis. Cells were subjected to fluorescent microscopy with a Zeiss Axiovert 200 M inverted fluorescent microscope and mitotic index quantified using Metamorph software (Universal Imaging Systems). Data is expressed as the percentage of cells positive for histone-H3 staining, relative to the number of cells present. Mean data (with S.E.M) is shown, where 8 wells are sampled 9 times for each knockdown condition. iii) The average number of cells per field of view is also shown, as a measure of cell proliferation at 72 h.

### DETAILED DESCRIPTION OF THE INVENTION

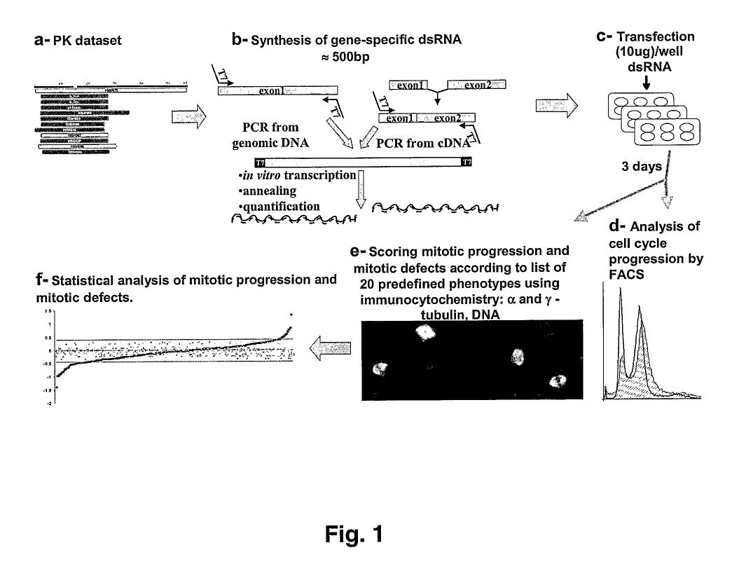

Our strategy was to transfect dsRNA for each of the predicted 228 kinase genes into S2 cells and monitor the effect 72 hours later, a time sufficient to deplete most cell cycle proteins and reveal cellular phenotypes^{10,11} (Methods and FIG. 1).

We considered how to counter artifacts that might arise in such a survey. To avoid scoring background cell cycle defects in the S2 line⁶ we were conservative in the definition of phenotypes and only considered as positives those kinases that consistently showed a FACS phenotype in 3-6 independent experiments or a quantitative mitotic phenotype in 2 independent experiments. The second possible artifact is lack of specificity and effectiveness of the technique. In Drosophila cells RNAi does not seem to present the same problems regarding specificity and effectiveness that mammalian systems do⁵⁴. However, as a check on specificity, we have used different primer pairs to produce dsRNA for a quarter of the kinases that showed a cell cycle phenotype and were able to replicate our results. Additionally, in the case of CDK4, SNF1, CKIIα and Pvr kinases, we also carried out RNAi with positive regulators of their activity and found similar phenotypes (see main text). It is also our experience that RNAi is usually highly effective in cultured Drosophila cells and this was confirmed by our ability to identify the majority of known cell cycle kinases. We also considered whether some kinases might be not expressed in S2 cells leading us to miss cell cycle functions. However, there is very little redundancy of kinases in the Drosophila genome and we would expect the majority of cell cycle kinases to be expressed in these cells.

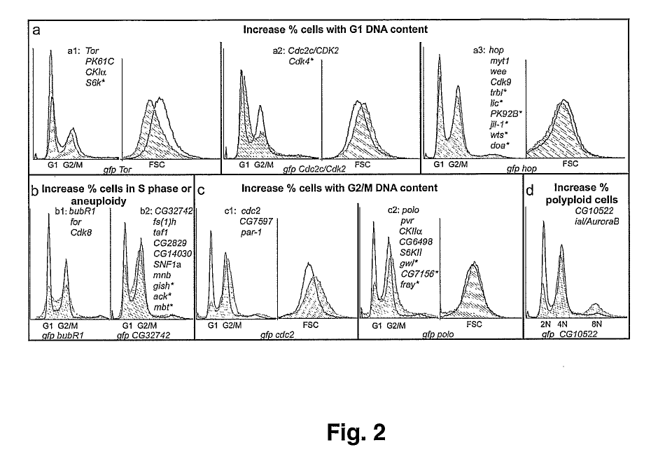

Flow cytometry revealed delays in progression through specific cell cycle stages, which in some cases associated with aneuploidy, polyploidy or cell death, following down-regulation of 42 protein kinases (18% of the kinome). These fall into four broad clusters, taking into account also effects on cell size, a parameter used classically in defining phenotypes of cell division cycle (cdc) mutants in the yeasts (FIG. 2).

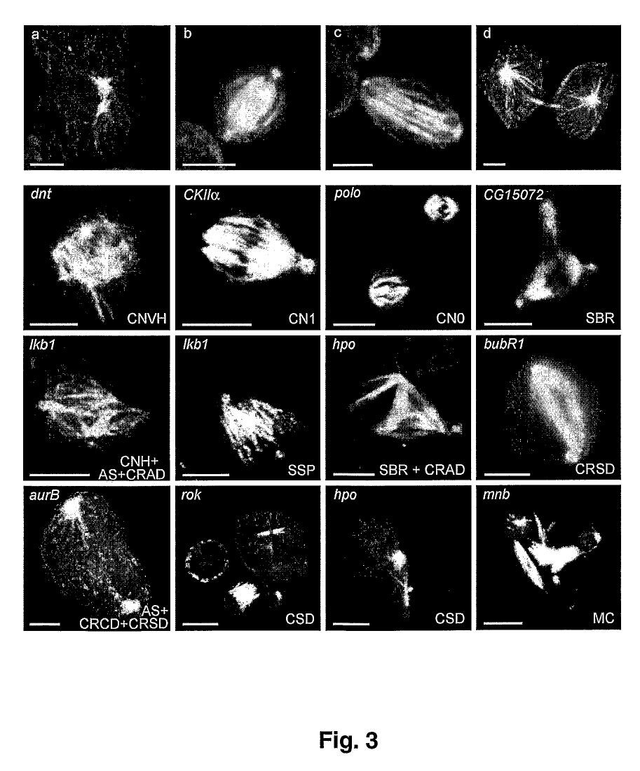

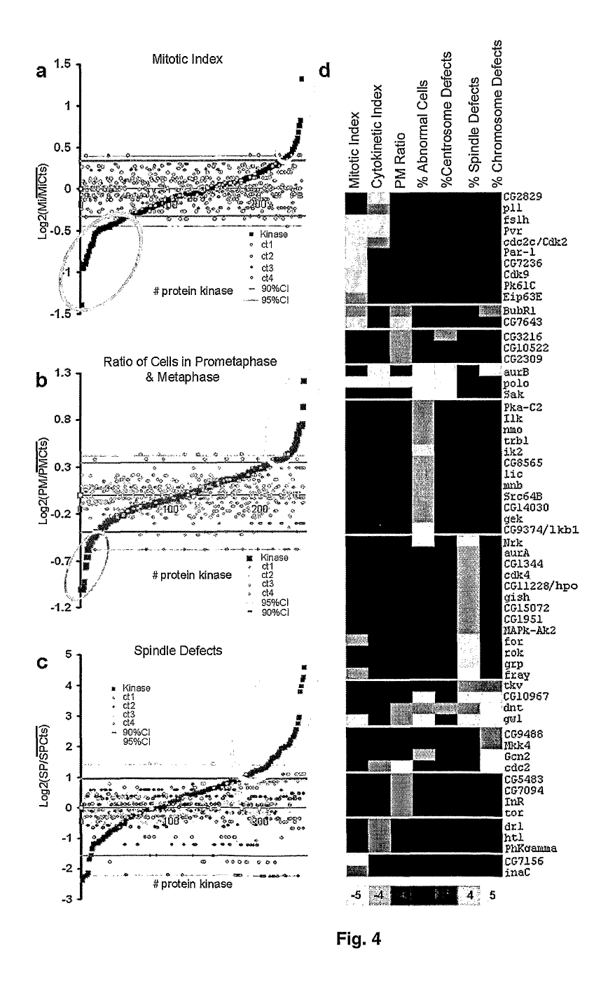

Flow cytometry does, however, miss some mitotic defects. RNAi on Aurora A, for example, a gene that has well-defined centrosomal and spindle assembly functions, did not reveal a phenotype by flow cytometry. This is probably because cultured Drosophila cells are tolerant of both supernumerary centrosomes⁶, and their complete absence¹². We therefore carried out RNAi on the 228 kinases and blindly quantitated 20 parameters including centrosomal, spindle and chromosomal defects, the proportions of cells in the classical mitotic stages, and mitotic index (FIG. 3). Kinases were ranked according to each of their phenotypic scores (FIG. 4a-c). We defined an RNAi phenotype only when the phenotypic score was significantly different from controls in two independent experiments. According to this definition 60 kinases showed a mitotic phenotype (FIG. 4d).

In total 80 kinases showed cell cycle progression and/or mitotic defects (FIG. 2 and FIG. 4). These enzymes were grouped according to their phenotype and/or functional information from other systems (Table 2). Previously known cell cycle regulatory protein kinases (21 enzymes, highlighted in Table 2) showed functions similar to corresponding fly mutants or studies in other organisms, validating the approach.

### Relations Between Signal Transduction, Stress Response and Cell Cycle

Depletion of a number of protein kinases, known to respond to growth factors and environmental stress, including members of NF-κB, JNK/p38 and JAK/STAT signalling pathways, led to cell cycle defects, indicating that extracellular conditions bear directly on cell cycle progression. One cluster of these kinases showed an increase in cells in G1 with no significant change in cell size following RNAi (Table 2, group 1a). Within this cluster were PK92B and licorne, two stress response enzymes in MAPK pathways (Table 2). In mammals, depending on the cell type, p38 MAPKs can function either to stimulate or inhibit cell proliferation through regulation of cyclin D expression¹³. Another enzyme present in this cluster is Doa, a LAMMER family kinase. Recent genetic evidence indicates that Drosophila Doa mutants show disrupted endoreplication of nurse cell chromosomes and fail to sustain condensation of the oocyte DNA¹⁴. Further studies are required to determine whether this protein kinase has comparable roles in the more conventional cycles of S2 cells. Two other kinases in this group have been implicated in NF-κB activation: Jil1, known to regulate chromatin structure, and Pelle, the counterpart of mammalian Interleukin 1 Receptor Associated Kinase (IRAK).

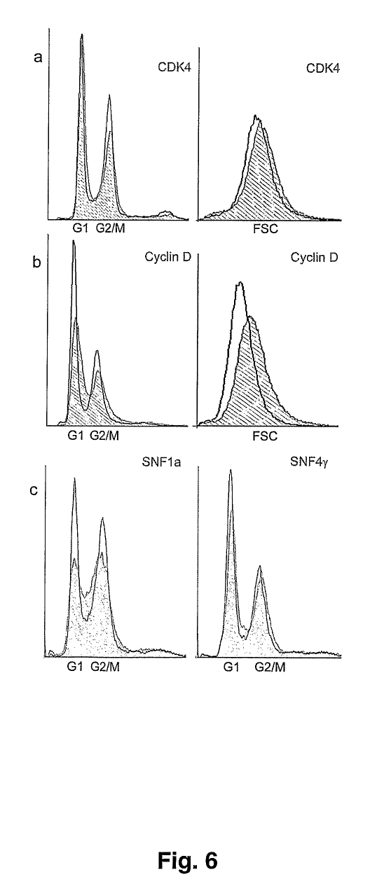

Coupling of JAK-STAT signalling to proliferation in the S2 cell line was suggested by the accumulation of cells with G1 DNA content following down-regulation of the Hopscotch JAK Kinase. Consistent with genetic interactions suggesting that Cdk4 functions downstream of hopscotch, we found cells of increased size also accumulated in G1 following either RNAi for CDK4 (FIG. 2a2) or its putative partner, cyclin D (FIG. 6). However, Drosophila Cdk4 imaginal disc clones show a longer cell cycle with no change in cell cycle profile and size distribution in FACS, implicating CDK4 in the regulation of growth rate¹⁵. Together this suggests that the relative role of CDK4 in regulating cell cycle may depend upon the cell type, also suggested by another recent study¹⁶.

A broad spectrum of other phenotypes was seen following the down regulation of several signaling pathways; various mitotic phenotypes for Nemo and Ik2 (Table 2, group 5), chromosomal alignment defects for Mkk4 (Table 2, group 5), mitotic defects and/or delays in the progression through cytokinesis after down-regulation of several receptor-like kinases (Table 2, group 1b). It will be of future interest to determine whether these phenotypes indicate other primary functions for these enzymes or secondary effects of the signalling pathways on cell cycle progression.

### Nutrient Sensing, Cell Growth and Cell Cycle Progression

Most kinases in the cluster whose down-regulation led to an increase in the proportion of small G1 cells were known members of the TOR-PDK1-S6K system (FIG. 2a1, Table 2, group 2), conserved from yeast to mammals, consistent with their known functions in sensing nutrients and regulating cell growth. S6K is the effector kinase that phosphorylates ribosomal protein S6 to modulate translation. It can be activated either by nutrient sensing through Tor kinase or Ptd Ins 3,4,5P(3) dependent kinase (PDK; Pk61C in Drosophila). The latter usually responds to receptor tyrosine kinase (RTK) signalling, for example the insulin receptor, through PI-3 kinase¹⁷. What the receptor tyrosine kinase might be in S2 cells is not clear, as InR RNAi itself led only to a weak mitotic phenotype. Down-regulation of only one other protein kinase, CKIα, led to G1 delay with small cells, suggesting a novel function for this enzyme in the pathway.

We also found spindle and chromosomal alignment defects following down-regulation of Gcn2, an enzyme that phosphorylates eIF2 to impede translation in cells deprived of essential amino acids. Down-regulation of TOR by rapamycin induces the dephosphorylation and activation of Gcn2¹⁸. Thus two major pathways of nutrient control of gene expression each seems to show links not only with each other but also with cell cycle regulation emphasizing the need to coordinate these processes.

### Progression Into and Through S Phase

In addition to the increase in G1 cells following down-regulation of known G1/S regulators, including Cdk2 and Cdk4 (FIG. 2a2), we identified several transcriptional regulators implicated in the cell cycle and wider functions. These included Cdk8 and Cdk9, both known to phosphorylate RNA polymerase II.

S phase defects indicate that CG32742 is the potential counterpart of the budding yeast Cdc7, a conserved kinase that phosphorylates Mcm proteins at replication origins. S phase defects coupled with lower mitotic and cytokinetic indices and cell death were also seen following down-regulation of CG2829, the Drosophila counterpart of Tousled kinase (FIG. 4d and Table 2, group 3), a conserved enzyme that regulates chromatin assembly following DNA replication and a target of the DNA damage checkpoint. This is consistent with the tousled mutant phenotype: embryos of tousled show arrest of cell cycle progression in interphase, followed by apoptotic cell death¹⁹.

### Protein Kinases Inhibiting or Promoting the G2/M Transition

Identification of the known major genes that regulate the G2/M transition provided additional validation of our screen (Table 2, group 4). Knockdown of the major mitotic kinase, Cdk1, led to the expected increase in large G2 cells (FIG. 2c1). We also identified the CDK1 inactivating kinases Dwee1 and Myt1 and the Tribbles kinase that induces proteolysis of String, the CDK1 activating protein phosphatase. Down-regulation of this group accelerated G2 thus shifting more cells into G1 (FIG. 2a3). The Wts/Lats tumour suppressor kinase, another negative regulator of Cdk1, also led to an increase in G1 cells following RNAi. Downregulation of S6KII led to an increase in G2/M cells, in agreement with reports that its counterpart, the Xenopus p90^{rsk}, inactivates Myt1 during oocyte maturation²⁰. We also place a cdc2-related kinase, CG7597, into this category because its down-regulation resulted in a low mitotic index (FIG. 4d) with an increase in larger G2 cells (FIG. 2c1).

New G2 functions were identified for Taf1 and Fs(1)h kinases, previously shown to be transcriptional regulators and likely to be chromosomally associated since they contain bromodomains. Indeed, it has been reported that Taf1 is required for transcriptional activation of the string gene (cdc25)²¹. One possible human counterpart of Fs(1)h is Brd4 which has been suggested to be required for G2/M progression; another is Brd2/RING3 which participates in transactivation of promoters dependent on E2F. In genetic agreement Drosophila E2F1 has been shown to modulate the expression not only of genes required for G1/S but also of string²².

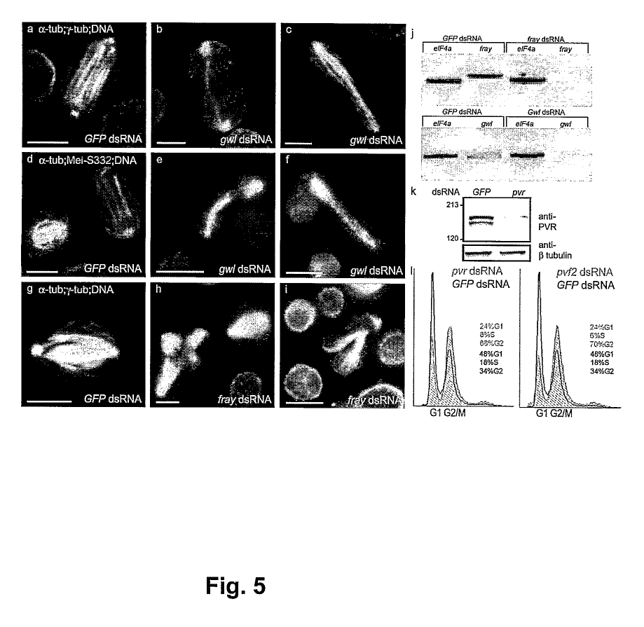

Unexpectedly, down-regulation of the Pvr receptor tyrosine kinase led to an increase in G2 cells (FIG. 5k,l), positive for cyclin A and B (not shown), and to a low mitotic index (FIG. 4d). Pvr is the counterpart of mammalian PDGF and VEGF receptors and signals border cell migration in oogenesis, a role that it shares with EGFR. RNAi against one of its ligands (pvf2), but not two others (pvf3 and pvf1), resulted in a similar phenotype (FIG. 5l). This suggests that S2 cells autoregulate proliferation through a signalling pathway effective at G2 and seems at odds with the generally accepted view that extracellular signalling directs cells through G1. However, String is highly regulated during Drosophila development: wing disc cells spend an increased proportion of time in G2 as they develop and differentiating photoreceptor cell preclusters trigger increased levels of String in neighbouring cells²³. Thus G2 delay following down regulation of Pvr signalling could reflect a specific property of insect cells. It might also exemplify wider possibilities for the regulation of G2 progression by external signalling. Indeed, recent characterisation of the mouse MKK7 knockout phenotype also suggests that signalling through the JNK pathway couples environmental cues to G2/M regulation²⁴.

### LKB1 Signalling has Pleiotropic Roles in Cell Cycle Progression

Our screen has identified new roles for several members of the LKB1 protein kinase cascade. Over-expression of wild-type, but not kinase-inactive, LKB1 can suppress the growth of some human cancer cell lines apparently through p53-mediated expression of the p21 cdk inhibitor²⁵. Recently it has been shown that LKB1 can activate some 13 members of the AMPK subfamily²⁶. We found cell cycle phenotypes with LKB1 and with three putative LKB1 targets, CG15072, SNF1A and Par1. Downregulation of either CG15072 or LKB1 showed strong effects on spindle morphology (FIG. 3). By contrast, RNAi of the AMP-activated protein kinase, SNF1A (Table 2, group 3), led to pleiotropic defects including an increase in S phase cells, also seen following down-regulation of its regulator, SNF4γ (FIG. 6). This suggests a direct link between sensing cellular energy by AMP-regulated protein kinase and cell cycle progression. Down-regulation of Par1 resulted in a striking increase in G2 cells. Since Par1 is better known as an enzyme that cooperates with LKB1 to regulate cellular polarity, this highlights the need for further studies of this network in cell cycle progression.

### Mitotic Functions

Among the enzymes whose depletion led to mitotic defects was the well-characterised Polo kinase. polo RNAi led to the typical features of strongly hypomorphic polo mutants²⁷: a dramatic increase in metaphase-arrested cells (FIG. 4d) and a ten-fold increase in spindles with no γ-tubulin at the poles (FIGS. 3 and 4d). This reflects the role of Polo in regulating centrosome maturation and the metaphase-anaphase transition^{4,27}. The Aurora A kinase also fell into this group as did several other kinases showing equal or greater RNAi spindle defects. Many of these kinases have not previously been studied in Drosophila and our attempts to find mammalian counterparts by sequence homology also identified poorly characterised kinases (Table 2). Of these the CG1951 and CG6498 kinases are particularly interesting since their putative mammalian counterparts are associated with centrosomes and with the manchette microtubules of spermatids (Table 2, group 5). RNAi on CKIIalpha led to an increase in G2/M cells and mitotic defects including spindles with a single centrosome (FIG. 3). An increase in centrosomal abnormalities was also observed with RNAi of its regulator CKIIβ (not shown). While this may indicate a direct mitotic function, the known pleiotropy of CKII²⁸ makes it difficult to exclude indirect effects.

We also found mitotic defects following down-regulation of two Ste20-related kinases: abnormal spindles and abnormal chromosome behaviour for fray RNAi (FIG. 5h, i) and an increase in G2/M cells, and possibly aneuploidy, following knockdown of mushroom bodies tiny (mbt). The Mbt kinase has been shown to localise to adherens junctions in a cdc42-GTP dependent manner²⁹. It is not clear what the precise vertebrate counterpart of Mbt is, but one possible orthologue, PAK5, regulates both the actin and tubulin cytoskeletons³⁰.

The role of the actin cytoskeleton in microtubule attachment to kinetochores³¹ and early mitotic events, such as spindle positioning and assembly³², has only recently become apparent. We found suggestions for roles of the actin cytoskeleton in mitosis from RNAi of the putative actin cytoskeleton regulators, Integrin linked kinase (Ilk), Src64B, and Genghis Kahn (gek) (Table 2, group 5). Knock-down of gek, an effector of cdc42 known to regulate actin polymerisation in the developing egg chamber³³, led to the formation of abnormal spindles with chromosome alignment defects (Table 2, group 5).

Finally, defects in spindle morphology and chromosome congression and/or segregation following greatwall RNAi suggested new mitotic functions for this kinase (FIGS. 4d, 5b, c). Spindles of metaphase length had uncongressed chromosomes and cells with elongated anaphase-like spindles had unequal numbers of chromosomes segregated to the poles after gwl RNAi (FIG. 5c). To determine whether lagging or pole associated chromosomes were separated chromatids, we examined the distribution of the Mei-S332 protein³⁴. In control cells, Mei-S332 is lost from centromeres as sisters separate at the metaphase-anaphase transition (FIG. 5d). In contrast, Mei-S332 was not lost from centromeres in comparable gwl RNAi cells (FIG. 5e, f). These results suggest that gwl functions either in regulating the attachment of sister kinetochores to opposite spindle poles to enable sister separation, in breaking sister chromatid cohesion, or both. We did not observe the pronounced chromosome condensation defects recently described in greatwall Drosophila mutants³⁵.

### Spindle Integrity Checkpoint

The spindle integrity checkpoint delays anaphase until all chromosomes are correctly aligned with sister kinetochores attached to opposite poles and under tension³⁶. Its failure leads to premature anaphase, therefore to a lowered mitotic index with lagging chromatids^{36,37}. Our survey identified such phenotypes after RNAi of the spindle integrity checkpoint kinases BubR1³⁸ and CG7643, the Drosophila counterpart of Mps1 kinase (FIGS. 3, 4d; Table 2, group 6). Surprisingly, depletion of the Bub1 checkpoint kinase³⁸ led to no change in mitotic index or of the proportion of cells passing through metaphase. Bub1 RNAi also does nat compromise anaphase timing in mammalian cells³⁹; this is consistent with the observation that BubR1 and Mps1, but not Bub1, dynamically exchange from the kinetochore to delay anaphase onset⁴⁰.

The report that Mps1 is also required for centrosome replication⁴¹ in human cells is controversial⁴². We saw no indication of this following CG7643 RNAi in S2 cells, but as we have noted above, these cells tolerate considerable variation in centrosome number^{6,12}. If Mps1 is required for centrosome duplication in some aspect of Drosophila development, the requirement is not seen in this cell line.

### Late Mitosis and Cytokinesis

Within this group Hippo, a recently characterised regulator of apoptosis and cell cycle exit⁴³ showed notable spindle and central spindle defects (FIG. 3). We also identified the major kinases already known to regulate cytokinesis (Table 2, group 7). These include the passenger kinase Aurora B¹¹ as well as two enzymes that phosphorylate the myosin regulatory light chain, the Rho-dependent and Citron kinases^{44,45}. Down regulation of Rho-kinase led to central spindle defects (FIG. 3) with no increase in polyploid cells, suggesting that cells recover and complete cytokinesis. Depletion of citron kinase (CG10522) resulted in the formation of many binucleate cells (FIG. 2d), as previously reported⁴⁵.

### CONCLUSIONS

Our study has identified new cell cycle protein kinases and assigned new cell cycle functions to previously known enzymes. The G2 arrest seen following down-regulation of the PDGF/VEGF-related receptor, PVR, exemplifies one such new role. The survey further highlights those aspects of cellular physiology regulated by protein phosphorylation that are intimately linked to cell cycle progression. These include external signalling from growth factors or nutrients, cellular responses to stress and regulation of cell growth. We also found new mitotic functions for enzymes predicted to regulate cytoskeletal elements, those that link extracellular signalling and actin cytoskeleton regulation with the G2/M transition and mitosis are of particular interest. Further studies of those kinases should shed more light on these and similar findings by others^{24,31,32}. Furthermore, the assays developed and the phenotypes identified could be used as a platform for identification of interacting genes.

Although we adopted conservative criteria, we identified most previously known cell cycle kinases. We found phenotypes consistent with equivalent mutants in the fly and other organisms. This validates our approach and gives confidence that the approach has identified the great majority of kinases that regulate cell cycle progression in S2 cells. The ability of this line to tolerate defects such as abnormal centrosome numbers, however, means that we may have overlooked kinases that are absolutely essential in the whole organism. We were, for example, unable to assign a cell cycle function to the Drosophila counterpart of the human Nek2 kinase. Only when we carefully examined this RNAi phenotype in separate experiments were we able to detect a very weak phenotype affecting centrosome integrity⁴⁶. Nevertheless, the low degree of redundancy in the fly genome does facilitate identification of most cell cycle functions and their high conservation suggests that the study of human counterparts will benefit the understanding and treatment of proliferative disease.

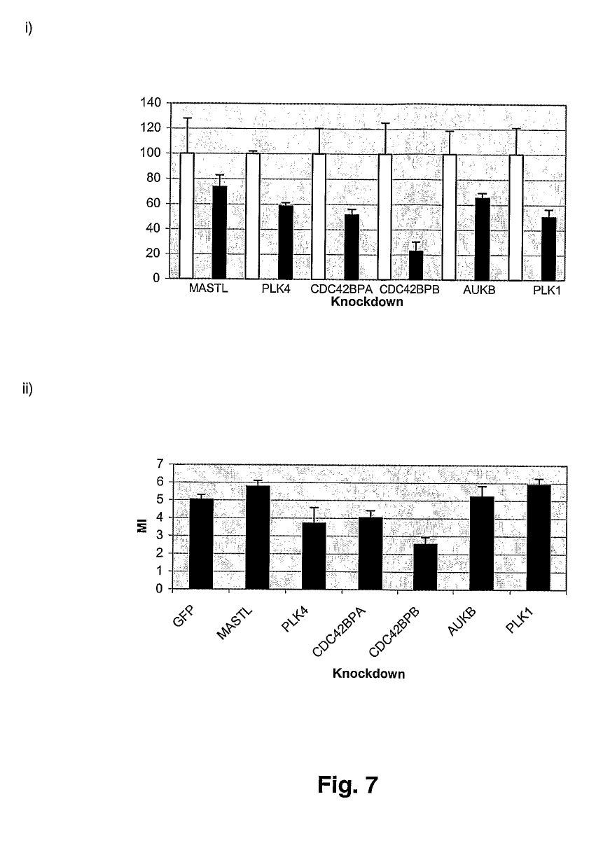

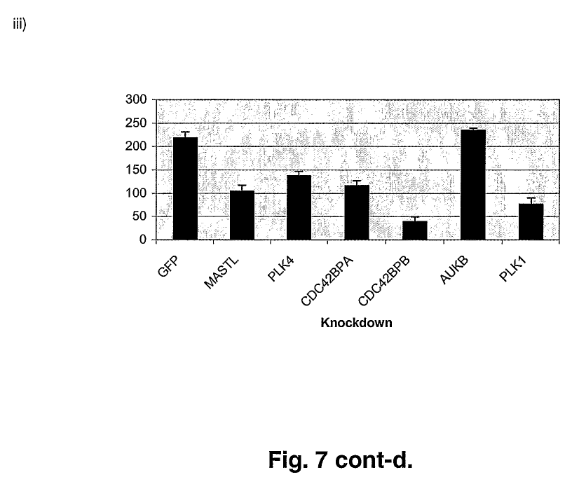

As validation of this we carried out transfection of human cancer cells (HeLa) with siRNAs to mediate RNA interference against four novel human kinase counterparts (MASTL (orthologue of gwl), PLK4 (orthologue of SAK), CDC42BPA and CDC42BPB (both orthologues of gek); see Table 1 for accession numbers). We also carried out RNA interference on the human counterparts of Drosophila Polo kinase and Aurora B kinase as controls. We assessed the level of knock-down of mRNA levels by quantitative PCR on reverse transcribed mRNA (QRT-PCR; FIG. 7i), the mitotic index by phospho-histone H3 staining (FIG. 7ii); and the effect on cell proliferation by cell counts after 3 days (FIG. 7iii). We show that down-regulation of all four human protein kinases results in reduced cell proliferation or survival. Control RNAis gave expected profiles.

TABLE 2

| Possible | | Putative | Previously Known Functions² | RNAi Phenotype in |
|---|---|---|---|---|
| Role | FlyBase Name | Orthologues¹ | (Human, Drosophila, C. elegans, S. pombe, S. cerevisae) | Current Study³ |
| Signal | Pk92B | HS-ASK1/MEKK5 | Activates Jun in cytokine and stress induced apoptosis | G1+ |
| transduction & | lic | HS-MAP2K3/6 | Phosphorylates p38MAPK; asymmetric development of the egg | G1+; ABN(3) SP (2) |
| stress | Doa | HS-CLK2/3/4 | Lammer dual specificity kinase 2; meiotic progression | G1+ |
| response | JIL-1 | HS-RPS6KA5/4 | Phosphorylates Histone H3; activation of NF-κB; chromatin | G1+ |
| 1a | | | structure | |
| | hop | HS-JAK2/3 | JAK-STAT signalling; proliferation; interacts genetically with | G1+ |
| | | | CDK4 | |
| | pll | HS-IRAK1 | Activation of NF-κB and MAPK pathways (JNK/p38); immunity | CYT(−4) |
| | tor | HS-RET* | Mutants-disruption in anterior/posterior axis; activates ras and | PM(−3) |
| | | | STAT | |
| 1b | drl | HS-RYK | ReceptorTK; axon pathfinding; Wnt receptor signalling pathway | CYT(3) |
| | htl | HS-FGR2/3/1/4 | FGF receptor; interacts with ras; cell migration; upstream of pbl | CYT(3) |
| Cell Growth/ | Tor | HS-FRAP1 | Regulates G1/S transition; mutant cells-smaller & arrest in G1 | G1+; size −; CYT(−2) |
| G1 | Pk61C | HS-PDK1 | Activation of p70S6K; upstream effector of S6k; Mutant cells- | G1+; size −; MI(−4) |
| | | | smaller | |
| 2 | S6k | HS-RPS6KB1/2 | Cell proliferation& growth; interacts Pk61C, Tor; Mutant cells- | G1+; size − |
| | | | smaller | |
| | Cklα | HS-CSNK1A1 | Inhibits JNK cascade; armadilllo degradation; induced after DNA | G1+; size − |
| | | | damage | |
| | InR | HS-IGF1R | Signals to MAPK/ras & PI3K; mutants-long lived & smaller body | PM(−3) |
| G1/S and S | cdc2c | HS-CDK2 | G1/S & S phase progression; G1/S & S phase progression | G1+; size+; MI(−4) |
| | | | | CYT(−3) |
| | Cdk4 | HS-CDK6/4 | G1/S transition; Cell growth | G2/M−, S+; size +; SP(3; |
| | | | | SBR) |
| 3 | Cdk8 | HS-CDK8 | Regulation of RNA polymerase 2 | S+; G1 = G2/M |
| | Cdk9 | HS-CDK9 | Regulation of RNA polymerase 2 | G1+; MI(−4) |
| | CG32742 | HS-cdc7 | DNA replication | S+, G2/M+ |
| | CG2829 | HS-TLK1/2 | Chromatin assembly; nuclear divisions & chromatin assembly & | S+; G2/M+; CYT(−4) |
| | | | cell viability | MI(−2) |
| | SNF1A | HS-AMPK2; | Metabolic stress response; regulation of pol II and initiation | S+, G2/M+; ABN (2) CN |
| | SC-SNF1 | | of meiosis | (2) |
| G2/M | cdc2 | HS-CDK1 | G2 to M-phase transition/mitosis | G2/M+; size+; CYT(−3) |
| transition | | | | PM(4) CHR(5) |
| | Myt1 | HS-Myt1 | Negative regulator of CDK1; regulates mitotic entry | G1+ |
| | wee | HS-Wee1 | Phosphorylation of CDK1; Wee1p phosphorylates Cdc2p on Tyr15 | G1+ |
| | trbl | HS-trb2/1/SKIP3 | SKIP3-upregulated in tumours; Induces Proteolysis of string | G1+; ABN(3) SP(2) |
| | | | (cdc25) | |
| | wts | HS-LATS1 | Inhibits G2/M and promotes apoptosis; interacts with CycA and | G1+ |
| | | | cdc2 | |
| | S6kll | HS-RSK2/1/3/6 | Inactivates Myt1 (Xenopus laevis) | G2/M+ |
| | CG7236 | HS-CDKL1 | Involved in gliosis | MI(−5); multinucleate |
| | | | | cells |
| 4 | CG7597 | HS-CRK7; CE- | MPM-2 antigen; RNAi in vivo-slow growth | G2/M+; size +; MI(−2) |
| | | B0285.1 | | |
| | Eip63E | HS-PFTAIRE-1 | Embryonic and larval development | MI (−3) |
| | Taf1 | HS-Tafll250 | TATA box BP associated - induces G1 progression through p53; | S+, G2/M+; MI(−2) |
| | | | transcriptional activation of string/cdc25 | ABN(2) |
| | fs(1)h | HS-BRD4/MCAP or | BRD4 associates with chromosomes; G2/M function; RING3 | S+ and/or aneuploidy, |
| | | RING3* | trans-activates genes dependent on E2F | G2/M+; MI(−5) |
| | | | | CYT(−5) PM(2) |
| | Pvr | HS-VEGFR1/2/3 | Proliferation and cell migration; organisation of actin | S−, G2/M+; MI(−5) |
| | | | cytoskeleton | CYT(−4) |
| | par-1 | HS-MARK3 | Phosphorylates CDC25C; interacts genetically with lkb1- | G2/M+, size +; MI(−4); |
| | | | regulates polarity | ABN (2) |
| | Ack | HS-Ack1 | Effector of cdc42; dorsal closure; expressed in mitotic domains | S+; G2/M+ |
| Mitosis | polo | HS-plk1; SC-cdc5 | Multiple mitotic functions | G2/M+; MI(5) CYT(−5) |
| | | | | PM(5) ABN(5) |
| | | | | CN(5: CNO) SP(2) |
| | Sak | HS-SAK | Required for mitosis (Mus musculus) | ABN(5) CN(5) & AS |
| | aurA | HS-Aurora A | Entry in mitosis; defects in centrosome maturation and spindle | SP(3) |
| | | | formation | |
| | Ckllα | HS-CK2A1 | Phosphorylates p53; multiple signalling pathways; circadian | G2/M+; ABN(2: CN1 & |
| | | | clock | CRLC) |
| | CG7156 | HS-RSK-LIKE | Novel RSKL similar to JIL, S6K and S6KII | G2/M+; MI(4) SP(2) |
| 5 | Pka-C2 | HM-PKA-Cbeta*; SC- | Regulates mitotic progression through cdc20 | ABN (3) SP(2) CHR(2) |
| | | PKA1 or 2* | | |
| | lkb1 | HS-LKB1 | Tumour suppressor; activates 13 kinases of the AMPK subfamily; | ABN (4) CN & SP |
| | | | oocyte microtubule organization. | |
| | CG15072 | HS-KIAA0999/QSK | AMPK-related kinase activated by LKB1 | SP(3) & CRAD |
| | nmo | HS-NemoLK | Wnt signalling - polarization/rotation of cells - NF-κB interactor | ABN(3: SP(2), & CN & |
| | | | | CRLC) |
| | ik2 | HS-TBK1 | NF-κB signalling; NF-κB signalling - defense response. | ABN(4); SP(2) |
| | inaC | SC-PKC1 | Mutants show visual behaviour defects; morphogenesis checkpoint | MI(3) |
| | dnt | HS-RYK | Up-regulated in ovarian cancer; Interacts with drl | PM(3) ABN(3) CN(3) |
| | | | | SP(3) CHR(5) |
| | for | HS-PRKG1 | NO/cGMP/cGK signaling - negative regulator of cell | S+/aneuploidy; MI(3) |
| | | | proliferation; - response to hypoxia - behaviour | ABN(2) SP(4: SMO) |
| | CG3216 | HS-Atrial natriuretic | Responds to cGMP; inhibits proliferation | PM(3) CN(3) |
| | | peptide receptor* | | |
| | CG1951 | HS-KIAA1360/ | NTKL localises to centrosomes during mitosis | SP(3) |
| | | NTKL*; SC-SCY1 | | |
| | CG6498 | HS-MAST1 or 2 | Localises to spermatid manchette (Mus musculus); activates NF- | G2/M+; CRAD |
| | | | κB | |
| | Mkk4 | HS-MAP2K4 | JAK-STAT & JNK cascades-links stress response to cell cycle | CHR (3: CRAD) |
| Mitosis | MAPk- | HS-MAPKAPK2 | Activated in response to IFN in the p38 pathway | SP(3) & CRAD |
| | Ak2 | | | |
| | fray | HS-OSR1 or SPAK | Oxidative stress response; phosphorylates PAK1; Nerve | MI(3); SP(5) & CRAD |
| | | | ensheathment | |
| 5 | mbt | HS-PAK7/5/4 | Cdc42/Rac interacting; cytoskeleton & photoreceptor | S+ and/or aneuploidy; |
| | | | development | G2/M+; CRAD |
| | Ilk | HS-ILK1/2; CE-ILK | Linkage of integrins to actin cytoskeleton; focal adhesions of | ABN(3: CHR(2; |
| | | | cytoskeleton | CRLC); AS) |
| | Src64B | HS-FYN* | Regulation of actin polymerisation; cell proliferation | MI(−2); ABN (3: |
| | | | | SP&CHR) |
| | gek | HS-CDC42BPB | Abnormal accumulation of F actin in oogenesis | ABN (3): AS & CRAD |
| | CG1344 | HS-Pace-1 | Cell spreading and motility - colocalises with ezrin in lamellipodia | SP (3) |
| | gish | HS-CK1G3; CE- | Glial cell migration; Mitotic spindle orientation; growth and | S+ and/or aneuploidy; |
| | | Y106G6E.6; SC- | division - cell morphogenesis and cytokinesis | G2/M+; MI(2) SP(3) |
| | | YCK1 or 2 | | |
| | gwl | HS-FLJ14813; SC- | Sporulation and meiosis; cek1 is suppressor of cut 8; chromosome | G2/M+; MI(4) PM(3) |
| | | rim15; SP-cek1 | condensation defects | ABN(5) SP(4) CHR(5; |
| | | | | CRSD) |
| | mnb | HS-DyrK1; CE- | Candidate target of Down's Syndrome; mutants have small | S+ and/or aneuploidy; |
| | | mbk1/2* | brains; spindle positioning and asymmetric cell division | G2/M+; ABN(3): AS |
| | CG2309 | HS-ERK8 | Activated by SRC | PM(3) |
| | CG10967 | HS-ULK2; CE-Unc- | Axon morphogenesis and elongation; may signal through ras | ABN(5); SP(4); CN(2); |
| | | 51; | | CHR(4) |
| | Gcn2 | HS-KIAA1338/GCN2 | Phosphorylates elF2alpha in amino acid deprivation; protein | ABN(3) SP(2) |
| | | | synthesis in stress response | CHR(5: CRAD) |
| | tkv | HS-BMPR1B | Type I TGFβ receptor; cell growth and division - anterior/posterior | ABN (2); SP (3); CHR |
| | | | patterning | (3: polyploid cells & |
| | | | | CRAD) |
| | Nrk | HS-MUSK | Muscle specific tyrosine kinase receptor; interacts with ras in | ABN(5)SP(4)CHR (2; |
| | | | oocytes | CRAD) |
| | CG8565 | HS-SRPK2*; CE- | Pre-mRNA splicing; SPK-1, required for embryogenesis | ABN(3)CN (2: CN1) |
| | | SPK-1* | and germline development | |
| | CG9488 | HS-DDR2* | Extracellular matrix remodelling | CHR (3) |
| Checkpoints | BubR1 | HS-BubR1; SC-Bub1 | Spindle assembly checkpoint; mutants show low MI and | Aneuploidy; MI(−3) |
| | | | premature mitotic exit | PM(−3) ABN(2) CHR(3: |
| | | | | CRLC & CSD) |
| | CG7643 | HS-TTK; SC-MPS1 | Spindle assembly checkpoint & centrosome duplication; | MI(−4) PM(−4) SP(2) |
| | | | duplication of SPB & spindle assembly checkpoint | |
| 6 | CG14030 | HS-BUBR1; SC-Bub1 | Spindle assembly checkpoint; Does not contain KEN box; | Aneuploidy; ABN (3; |
| | BUB1 | | functionally similar to Human Bub1 and SC Bub1 | CRLC & CSD) |
| | grp | HS-Chk1 | Replication and DNA damage (G2) checkpoint; cell cycle | SP (4); CRAD & CRLC |
| | | | coordination in syncytial embryo, mutant has defects in mitotic | |
| | | | entry | |
| Telophase & | CG7094 | HS-CSNK1A1* | Wnt signalling | PM (−3) |
| Cytokinesis | CG5483 | HS-KIAA1790 | Similarity to leucine-rich repeat kinase (LRRK1) | PM (−3); CN(2) |
| | hippo | HS-STK4/3*; SC- | Apoptosis; apoptosis and cell cycle exit; mitotic exit network | SP (3) & CRLC |
| | | cdc15* | | |
| 7 | aurora B | HS-AurB*; SC-Ipl1p* | Spindle assembly checkpoint & cytokinesis; chromosome | 8N peak; CYT(−5) |
| | | | condensation & cytokinesis | ABN(5) CN(4) CHR (4) |
| | CG10522 | HS-CIT | Cytokinesis; cytokinesis | 8N peak; PM(3) ABN(2) |
| | Rok | HS-ROCK | Cytokinesis; tissue polarity | ABN(2) SP(4: CSD) |
| | PhKγ | HS-PHKG1 | Metabolism; embryonic morphogenesis | CYT (3) CSD; CRLC |

80 protein kinases are grouped on the basis of phenotypes following RNAi (this study) and/or functional information from other systems. A putative human (HS) homologue and, in cases where known phenotypes are helpful in assessing function, potential counterparts from C. elegans (CE), budding yeast (SC) or fission yeast (SP) are suggested. ¹We obtained orthologues in the Inparanoid database⁴⁹ (confidence value=0.05 or higher). *The closest homologue from a BLAST⁵⁰ search (NCBI) is shown, when the orthology is not clear; ²Additional information, references and sources of information relating to the functions of orthologues for each individual protein are given in Supplementary Table 5; ³+/− indicates an increase/decrease in cell size or in the proportion of cells in a cell cycle compartment (G1, S or G2) in FACS analysis. The level of confidence for each phenotype corresponds to the scale indicated in FIG. 4. We have added additional information (in italics) to further describe the phenotypes observed: 2 and −2 indicate PS values falling out of the 85% CI. MI, mitotic index; PM, (prometaphase & metaphase ratio); CYT, cytokinetic index; ABN, all mitotic abnormalities; CN, centrosome abnormalities; SP, spindle abnormalities; CHR, chromosome abnormalities.

### Materials and Methods

### Double Stranded RNA Synthesis

DsRNA was made from genomic Drosphila DNA or cDNA as described in Bettencourt-Dias et al.⁴⁷ with an average length of 500 bp. The set of protein kinases was defined based on Morrison et al.⁴⁸ and Manning et al.⁹ and annotation in Flybase, using homologies with protein kinase catalytic sites.⁹ A list of primer pairs can be found in Table 3. dsRNA was analysed by electrophoresis in 1.5% agarose gels for quantification and to ensure that the RNA migrated as a single band.

Human orthologues of Drosophila kinases were identified as described in Table 1, and long double stranded RNA (dsiRNA) was synthesised from gene specific PCR products amplified to these targets with a T7 5′ sequence tag. The T7 oligonucleotides used for this study were towards;

MASTLI (forward
5′- taatacgactcactatagggggcagaaaggcggcaaattgt
and
reverse
5′- taatacgactcactatagggccaacgagctgataagcgataa),
PLK4 (forward
5′- taatacgactcactatagggcattcacactggtttggaagttg
and
reverse
5′- taatacgactcactatagggcccagggaccaaacatcaga),
CDC42BPA (forward
5- taatacgactcactatagggaggatcttattcgaaggctcat
and
reverse
5′- taatacgactcactataggggttagtggaccatcaacagttga),
CDC42BPB (forward
5′- taatacgactcactataggggcgctgcactacgcctttca
and
reverse
5′- taatacgactcactatagggatgggaactggaatcgctctt),
Aurora kinase B (forward
5′- taatacgactcactatagggcctctgggcaaaggcaagtt
and
reverse
5′- taatacgactcactatagggatgcgcccctcaatcatctct),
PLK1 (forward
5′- taatacgactcactatagggattgtgcttggctgccagtac
and
reverse
5′- taatacgactcactatagggtcgaaaaccttggtggaatgg)

PCR products were sequenced to confirm their identity. 1-2 μg of this DNA was used generate double stranded RNA in a Ribomax in-vitro T7 transcription reaction (Promega, Southampton, UK) according to the manufacturers instructions. 20 μg of long double stranded RNA for each gene, was exposed to recombinant DICER (Gene Therapy Systems, San Diego, USA) and the diced short interfering RNA (dsiRNA) was purified according to the manufacturers instructions.

### Cell Culture and Transfections

Drosophila S2 cells were cultured and transfected with 10 μg of dsRNA and 10 μl of Transfast (Promega) in six well plates as described in Supplementary FIG. 1 and in Bettencourt-Dias et al⁴⁷. Cells were harvested after 3 days.

Human HeLa cells were obtained from the European Collection of Cell Culture (Porton Down, Salisbury, Wiltshire, UK, ECACC No 93021013) and were used in experiments from passage 12-20 without noticeable changes in their morphology. HeLa cells were maintained in DMEM, supplemented with 10% batch tested fetal calf serum, 2 mM Glutamine, 1 mM non-essential amino acids, 100 μg/ml penicillin and 100 U/ml streptomycin. Cells were harvested every 3 or 4 days using a trypsin/1 mM EDTA seeding routinely at 1:6. All cell culture reagents were from Invitrogen (Paisley, UK), and all plasticware was from Beckton and Dickenson (Oxford, UK).

HeLa cells were prepared for transfection by seeding at 1×10⁴ per well of a 24 well plate, 24 hours prior to transfection. Cells were transfected with 50 ng (approx. 20 nM) dsiRNA and 0.45 μl TransFast (Promega), prepared according to the manufacturers instructions. Under these conditions we routinely observe transfection efficiencies of at least 80%, when FITC labelled siRNA (Dharmacon, Lafayette, CO USA) is transfected, and cells are harvested 24 h later and analysed on a BD LSR1 fluorescent activated cell sorter (BD Biosciences, Cowley, Oxford, UK)

### Western Blotting and RT-PCR

For protein analysis, an aliquot of the cells was resuspended and boiled in Laemmli buffer. Standard procedures for Western Blotting were used (see Supplementary Methods for details on Antibodies used). For RT-PCR analysis from Drosophila cells RNA was extracted using the Qiagen Rneasy Protect Mini Kit and RT-PCR was performed using the SuperScript First Strand Synthesis System according to manufacturer's instructions (Invitrogen).

For human cells, HeLa cells exposed to dsiRNA/lipid complexes for 4 hours, and cultured for a further 20 hours, were then harvested in 200 μl of Trizol (Invitrogen). RNA was purified according to the manufacturers instructions, and cDNA synthesised using Cells to cDNA kit (Ambion, Huntingdon, Cambridgeshire, UK) according to the manufacturers instructions. cDNA was then used in a quantitative RT-PCR reaction using Syber Green reaction mix (Applied Biosystems, Warrington, Cheshire) with appropriate forward and reverse oligos;

MASTL (forward
5′-catattaaactgacggatttggcc
and
reverse
5′-ggccaaaatccgtcagtttaatatg)
PLK4 (forward
5′-aggatcatttgctggtgtctacag
and
reverse
5′-gaaggatgtttcaattggcaatgtattttc)
CDC42BPA (forward
5′-gtacctccttgatggtgggtttaa
and
reverse
5′-tggacaagtggttggagcttt)
CDC42BPB (forward
5′-acctatgggaagatcatgaacca
and
reverse
5′-atgaggtccttcgcttcttcag)
AURKB (forward
5′-gcagaagagctgcacatttgac
and
reverse
5′-ccatggcagtacattagagcatct)
PLK1 (forward
5′-aacggcagcgtgcagatc
and
reverse
5′-ggtcacggctgccatcag).

QRT-PCR was performed on a Prism 7000 (Applied Biosystems) and actual amounts of target mRNA quantified after standardisation with ribosomal RNA. This was determined for each cDNA sample using Ribosomal RNA Control Reagents with VIC probe, and Taqman Universal PCR Mix (Applied Biosystems) according to the manufacturers instructions. For convenience, data is finally represented as percent of knockdown relative to controls, which were cells transfected with dsiGFP.

### Immunofluorescence Analysis

S2 cells were harvested 3 days after transfection, plated on glass coverslips and fixed 1 hour later in 4% formaldehyde in PHEM buffer (60 mM Pipes, 25 mM Hepes, 10 mM EGTA, 4 mM MgCl2). Cells were permeabilised and washed using PBST (PBS containing 0.1% Triton X-100 and 1% BSA). DNA was stained by TOTO3-iodide (Molecular Probes) or DAPI. Vectashield mounting medium H-1200 was purchased from Vector Laboratories. Counts were performed blindly by giving coded numbers to control and sample slides. 1000-3000 cells were scored per slide (comprising at least 60 mitotic cells). Cells were categorised according to phase of mitosis and to centrosome, spindle and DNA morphology and assigned to one of 20 potential mitotic phenotypic abnormalities (see supplementary Table 3), coded to facilitate computer analysis of the data. A ZEISS Axiovert 200M microscope was used for the countings. Data was then inserted into a datasheet (see supplementary Table 4 for downloadable datasheet) for analysis. Two datasets were obtained for each kinase, from two independent experiments. Seven phenotypic parameters (mitotic index, cytokinetic index, PM ratio, percentage of mitotic defects, percentage of centrosome defects; percentage of spindle defects and percentage of chromosome defects) were compared across the whole dataset. Details of the statistical analysis can be found in Supplementary Materials and Methods. Images were acquired using a confocal scanning head (model 1024; Bio-Rad Laboratories) mounted on an Optiphot microscope (Nikon) and prepared for publication using Adobe Photoshop®.

### Analysis of Mitotic Index in Human Cells

Cells transfected with various dsiRNA's were also analysed at 72 h for mitotic index by fixation in 4% formaline, permeabilising in PBS and 0.1% Tx100 (PBST), blocking for 1 h with in PBST and 1% BSA. Cells were incubated overnight at 4 C with an anti-phospho-histone H3 primary antibody (Upstate, Milton Keynes, UK) at 1:500 and a secondary antibody (Rhodamine anti-rabbit, Jackson Luton, Beds, UK) at 1:200 for 1 h at RT, whilst washing in between with PBST. Finally cells were incubated with DAPI in PBS for 30 min and washed again prior to analysis. Cells were subjected to fluorescent microscopy with a Zeiss Axiovert 200 M inverted fluorescent microscope and mitotic index quantified using Metamorph software (Universal Imaging Systems). Data is expressed as the percentage of cells positive for histone-H3 staining, relative to the number of cells present. Mean data (with S.E.M) is shown, where 3 wells are sampled 9 times for each knockdown condition. iii) The average number of cells per field of view is also shown, as a measure of cell proliferation at 72 h.

### Flow Cytometry

For FACS analysis, 2 mls of cells were recovered 3 days after transfection and fixed in 70% ice-cold ethanol. For analysis of levels of cyclin A, B and phospho-histone H3, cells were permeabilised and blocked using PBS with 1% BSA and 0.25% Triton X-100. All incubations with antibodies and wash steps were performed in PBS with 1% BSA. The cells were then incubated at 37° C. for 30 min in PBS containing 100 ug/ml RNAse (previously boiled for 5 min) and 100 ug/ml of propidium iodide before analysis. For analysis of DNA content we used a Becton Dickinson FACScan and a Becton Dickinson LSR and acquired data from 30000 cells. Results were analysed using Summits from Dako Cytommation and Multicycle®. At least 3 independent experiments were performed.

### Antibodies

Rat anti-tubulin antibody (clone YL1/2) and mouse anti-γ-tubulin clone (GTU88) were obtained from Sigma-Aldrich and anti-phospho-histone H3 from Upstate Biotechnology. Rabbit anti-cyclin B (Rb271) and rabbit anti-cyclin A (Rb270) have been described previously⁵¹. Anti-Mei-S332 antibody³⁴ was kindly given to us by Terry Orr-Weaver (MIT, USA). Rat anti-pvr antibody⁵⁷ was the kind gift of Pernille Rorth. FITC- or Texas red-conjugated goat anti-rat and anti-mouse were obtained from Sigma-Aldrich and Jackson Immuno Research Laboratories. Goat anti-rabbit Alexa-488 antibody (Molecular Probes) was used for FACS analysis. Peroxidase-conjugated goat anti-rabbit or anti-rat antibodies used in Western blotting were from Sigma-Aldrich.

### Statistical Analysis

Cells were categorised according to phase of mitosis and to centrosome, spindle and DNA morphology and assigned to one of 20 potential mitotic phenotypic abnormalities. Data was then inserted into a datasheet for analysis. Two datasets were obtained for each kinase, from two independent experiments. Seven phenotypic parameters (mitotic index, cytokinetic index, PM ratio, percentage of mitotic defects, percentage of centrosome defects, percentage of spindle defects and percentage of chromosome defects) were normalized and compared across the whole dataset. Normalised results from immunofluorescence countings are given as the Phenotypic Score (PS), which equals log₂ (x/ c̅ₜ) for all variables (with the exception of chromosomal abnormalities where results are given as log₂(100−x/100− c̅ₜ)), where x stands for the observed value (relative to total number of cells) and c̅ₜ for the mean value of the negative controls (relative to total number of cells) performed in the same experiment (same day). Confidence intervals were generated separately for each of the two repeats of experiments using negative control data only. Since there was a significant effect of the day on which the experiment was performed (due mainly to the age of the cells), we had to devise a specific bootstrap procedure for generating confidence intervals by resampling negative controls within days of experiment. The procedure works as follows: we first sample with replacement a batch of experiments t. We then sample with replacement nₜ+1 control data values, where nₜ represents the number of controls in batch t. One control data point is allocated to the numerator; the mean of the remaining nₜ data is computed and allocated to the denominator. The base 2 logarithm of this ratio is then computed. The procedure was repeated 2,000 times in order to produce the distribution that allowed us to compute the upper and lower confidence limits. We defined a “mitotic kinase” when PS values for at least one of the mitotic parameters fell out of the 90% CI in two independent experiments. To describe the strength of the phenotype, phenotypic confidence levels were used: at the extreme, arbitrary values −5 and 5 indicate respectively PS values outside the 99% CI at the lower or higher boundary in both experiments; −4 and 4 indicate PS values outside the 95% CI; −3 and 3 indicate PS values outside the 90% CI; −2 and 2 indicating PS values outside the 85% CI. “Cluster”⁵² and “JavaTreeView”⁵³ were used in clustering the kinases according to their mitotic phenotypes (FIG. 4d).

### REFERENCES

1. Pines, J. Cyclins and cyclin-dependent kinases: theme and variations. Adv Cancer Res 66, 181-212 (1995).

2. Nigg, E. A. Mitotic kinases as regulators of cell division and its checkpoints. Nat Rev Mol Cell Biol 2, 21-32. (2001).

3. Donaldson, M. M., Tavares, A. A., Hagan, I. M., Nigg, E. A. & Glover, D. M. The mitotic roles of Polo-like kinase. J Cell Sci 114, 2357-8. (2001).

4. Barr, F. A., Sillje, H. H. & Nigg, E. A. Polo-like kinases and the orchestration of cell division. Nat Rev Mol Cell Biol 5, 429-40 (2004).

5. Clemens, J. C. et al. Use of double-stranded RNA interference in Drosophila cell lines to dissect signal transduction pathways. Proc Natl Acad Sci USA 97, 6499-503. (2000).

6. Goshima, G. & Vale, R. D. The roles of microtubule-based motor proteins in mitosis: comprehensive RNAi analysis in the Drosophila S2 cell line. J Cell Biol 162, 1003-16 (2003).

7. Kiger, A. et al. A functional genomic analysis of cell morphology using RNA interference. J Biol 2, 27 (2003).

8. Lum, L. et al. Identification of Hedgehog pathway components by RNAi in Drosophila cultured cells. Science 299, 2039-45. (2003).

9. Manning, G., Plowman, G. D., Hunter, T. & Sudarsanam, S. Evolution of protein kinase signaling from yeast to man. Trends Biochem Sci 27, 514-20. (2002).

10. Somma, M. P., Fasulo, B., Cenci, G., Cundari, E. & Gatti, M. Molecular dissection of cytokinesis by RNA interference in Drosophila cultured cells. Mol Biol Cell 13, 2448-60. (2002).

11. Giet, R. & Glover, D. M. Drosophila aurora B kinase is required for histone H3 phosphorylation and condensin recruitment during chromosome condensation and to organize the central spindle during cytokinesis. J Cell Biol 152, 669-82. (2001).

12. Debec, A. & Abbadie, C. The acentriolar state of the Drosophila cell lines 1182. Biol Cell 67, 307-11 (1989).

13. Nebreda, A. R. & Porras, A. p38 MAP kinases: beyond the stress response. Trends Biochem Sci 25, 257-60 (2000).

14. Morris, J. Z., Navarro, C. & Lehmann, R. Identification and analysis of mutations in bob, Doa and eight new genes required for oocyte specification and development in Drosophila melanogaster. Genetics 164, 1435-46 (2003).

15. Meyer, C. A. et al. Drosophila Cdk4 is required for normal growth and is dispensable for cell cycle progression. Embo J 19, 4533-42 (2000).

16. Malumbres, M. et al. Mammalian cells cycle without the D-type cyclin-dependent kinases Cdk4 and Cdk6. Cell 118, 493-504 (2004).

17. Kozma, S. C. & Thomas, G. Regulation of cell size in growth, development and human disease: PI3K, PKB and S6K. Bioessays 24, 65-71 (2002).

18. Cherkasova, V. A. & Hinnebusch, A. G. Translational control by TOR and TAP42 through dephosphorylation of eIF2alpha kinase GCN2. Genes Dev 17, 859-72 (2003).

19. Carrera, P. et al. Tousled-like kinase functions with the chromatin assembly pathway regulating nuclear divisions. Genes Dev 17, 2578-90 (2003).

20. Tunquist, B. J. & Maller, J. L. Under arrest: cytostatic factor (CSF)-mediated metaphase arrest in vertebrate eggs. Genes Dev 17, 683-710 (2003).

21. Maile, T., Kwoczynski, S., Katzenberger, R. J., Wassarman, D. A. & Sauer, F. TAF1 activates transcription by phosphorylation of serine 33 in histone H2B. Science 304, 1010-4 (2004).

22. Neufeld, T. P., de la Cruz, A. F., Johnston, L. A. & Edgar, B. A. Coordination of growth and cell division in the Drosophila wing. Cell 93, 1183-93 (1998).

23. Lee, L. A. & Orr-Weaver, T. L. Regulation of cell cycles in Drosophila development: intrinsic and extrinsic cues. Annu Rev Genet 37, 545-78 (2003).

24. Wada, T. et al. MKK7 couples stress signalling to G2/M cell-cycle progression and cellular senescence. Nat Cell Biol 6, 215-26 (2004).

25. Tiainen, M., Vaahtomeri, K., Ylikorkala, A. & Makela, T. P. Growth arrest by the LKB1 tumor suppressor: induction of p21 (WAF1/CIP1). Hum Mol Genet 11, 1497-504 (2002).

26. Lizcano, J. M. et al. LKB1 is a master kinase that activates 13 kinases of the AMPK subfamily, including MARK/PAR-1. Embo J 23, 833-43 (2004).

27. Donaldson, M. M., Tavares, A. A., Ohkura, H., Deak, P. & Glover, D. M. Metaphase arrest with centromere separation in polo mutants of Drosophila. J Cell Biol 153, 663-76 (2001).

28. Litchfield, D. W. Protein kinase CK2: structure, regulation and role in cellular decisions of life and death. Biochem J 369, 1-15 (2003).

29. Schneeberger, D. & Raabe, T. Mbt, a Drosophila PAK protein, combines with Cdc42 to regulate photoreceptor cell morphogenesis. Development 130, 427-37 (2003).

30. Cau, J., Faure, S., Comps, M., Delsert, C. & Morin, N. A novel p21-activated kinase binds the actin and microtubule networks and induces microtubule stabilization. J Cell Biol 155, 1029-42 (2001).

31. Yasuda, S. et al. Cdc42 and mDia3 regulate microtubule attachment to kinetochores. Nature 428, 767-71 (2004).

32. Rosenblatt, J., Cramer, L. P., Baum, B. & McGee, K. M. Myosin II-dependent cortical movement is required for centrosome separation and positioning during mitotic spindle assembly. Cell 117, 361-72 (2004).

33. Luo, L. et al. Genghis Khan (Gek) as a putative effector for Drosophila Cdc42 and regulator of actin polymerization. Proc Natl Acad Sci USA 94, 12963-8 (1997).

14. Tang, T. T., Bickel, S. E., Young, L. M. & Orr-Weaver, T. L. Maintenance of sister-chromatid cohesion at the centromere by the Drosophila MEI-S332 protein. Genes Dev 12, 3843-56 (1998).

35. Yu, J. et al. Greatwall kinase: a nuclear protein required for proper chromosome condensation and mitotic progression in Drosophila. J Cell Biol 164, 487-92 (2004).

36. Cleveland, D. W., Mao, Y. & Sullivan, K. F. Centromeres and kinetochores: from epigenetics to mitotic checkpoint signaling. Cell 112, 407-21 (2003).

37. Jones, J. T., Myers, J. W., Ferrell, J. E. & Meyer, T. Probing the precision of the mitotic clock with a live-cell fluorescent biosensor. Nat Biotechnol 22, 306-12 (2004).

38. Logarinho, E. et al. Different spindle checkpoint proteins monitor microtubule attachment and tension at kinetochores in Drosophila cells. J Cell Sci 117, 1757-71 (2004).

39. Johnson, V. L., Scott, M. I., Holt, S. V., Hussein, D. & Taylor, S. S. Bub1 is required for kinetochore localization of BubR1, Cenp-E, Cenp-F and Mad2, and chromosome congression. J Cell Sci 117, 1577-89 (2004).

40. Howell, B. J. et al. Spindle checkpoint protein dynamics at kinetochores in living cells. Curr Biol 14, 953-64 (2004).

41. Fisk, H. A., Mattison, C. P. & Winey, M. Human Mps1 protein kinase is required for centrosome duplication and normal mitotic progression. Proc Natl Acad Sci USA 100, 14875-80 (2003).

42. Stucke, V. M., Sillje, H. H., Arnaud, L. & Nigg, E. A. Human Mps1 kinase is required for the spindle assembly checkpoint but not for centrosome duplication. Embo J 21, 1723-32 (2002).

43. Harvey, K. F., Pfleger, C. M. & Hariharan, I. K. The Drosophila Mst ortholog, hippo, restricts growth and cell proliferation and promotes apoptosis. Cell 114, 457-67 (2003).

44. Matsumura, F., Totsukawa, G., Yamakita, Y. & Yamashiro, S. Role of myosin light chain phosphorylation in the regulation of cytokinesis. Cell Struct Funct 26, 639-44 (2001).

45. D'Avino, P., Savoian, M. & Glover, D. Mutations in sticky lead to defective organization of the contractile ring during cytokinesis and are enhanced by Rho and suppressed by Rac. Journal of Cell Biology 5, 61-71 (2004).

46. Prigent, P., Glover, D. & Giet, R. The Drosophila Nek2 protein kinase is required for centrosome integrity and is able to regulate membrane addition during cytokinesis. Experimental Cell Research (2004).

47. Bettencourt-Dias M., Sinka R., Frenz L. & Glover, D. M. in Gene Silencing by RNA Interference: Technology and Application (ed. Sohail, M.) (CRC Press, 2004).

48. Morrison, D. K., Murakami, M. S. & Cleghon, V. Protein kinases and phosphatases in the Drosophila genome. J Cell Biol 150, F57-62. (2000).

49. Remm, M., Storm, C. E. & Sonnhammer, E. L. Automatic clustering of orthologs and in-paralogs from pairwise species comparisons. J Mol Biol 314, 1041-52 (2001).

50. Altschul, S. F. et al. Gapped BLAST and PSI-BLAST: a new generation of protein database search programs. Nucleic Acids Res 25, 3389-402 (1997).

51. Whitfield, W. G., Gonzalez, C., Maldonado-Codina, G. & Glover, D. M. The A- and B-type cyclins of Drosophila are accumulated and destroyed in temporally distinct events that define separable phases of the G2-M transition. Embo J 9, 2563-72 (1990).

52. de Hoon, M. J., Imoto, S., Kobayashi, K., Ogasawara, N. & Miyano, S. Inferring gene regulatory networks from time-ordered gene expression data of Bacillus subtilis using differential equations. Pac Symp Biocomput, 17-28 (2003).

53. Saldanha, A. J. Java treeview—extensible visualization of microarray data. Bioinformatics (2004).

54. Boutros, M. et al. Genome-wide RNAi analysis of growth and viability in Drosophila cells. Science 303, 832-5 (2004).

55. Giet, R., McLean, D., Descamps, S., Lee, M. J., Raff, J. W., Prigent, C. and Glover, D. M. (2002). Drosophila Aurora A kinase is required to localize D-TACC to centrosomes and to regulate astral microtubules. J Cell Biol 156, 437-51.

56. Rogers, S. L., Wiedemann, U., Stuurman, N. and Vale, R.

D. (2003). Molecular requirements for actin-based lamella formation in Drosophila S2 cells. J Cell Biol 162, 1079-88.

57. Duchek, P., Somogyi, K., Jekely, G., Beccari, S. & Rorth, P. Guidance of cell migration by the Drosophila PDGF/VEGF receptor. Cell 107, 17-26 (2001).

TABLE 3

List of Drosophila protein kinases studied in this
work (228) and primers used to synthesize
dsRNA. The set of protein kinases was defined based on
Morrison et al.⁴⁸, Manning et al.⁹ and annotation in FlyBase,
based on homologies with protein kinase catalytic sites⁹. All
primers led to the synthesis of a single band of dsRNA. Name
(as in FlyBase) and CG number are indicated. Two sets of
primers are indicated for genes for which different
transcripts exist or in cases where we rechecked the phenotype
observed. For the majority of these genes, DNA (with T7
polymerase binding site) amplified with these primers is
available from
http://www.hgmp.mrc.ac.uk/geneservice/reagents/products/
descriptions/Dros RNAi.shtml.

| | | |
|---|---|---|
| | | FORWARD SEQUENCE/ |
| NAME | CG | REVERSE SEQUENCE |

| | | |
|---|---|---|
| Abl | 4032 | TAATACGACTCACTATAGGGAGACACGGGCGATAGTCTGGAGCAGAGT/ |
| | | TAATACGACTCACTATAGGGAGACGGAATGGGGCTGGCCTTCGGATTT |
| Ack | 14992 | TAATACGACTCACTATAGGGAGATCTACTCGAATTTCAACCAGTCTCT/ |
| | | TAATACGACTCACTATAGGGAGATCATACCAAATACGATTCACCACAC |
| Aktl | 4006 | TAATACGACTCACTATAGGGAGATTACATCGGGTCATGCGCTTACGGAACA/ |
| | | TAATACGACTCACTATAGGGAGACACTTTCTTAACGCCGCTGCTATTA |
| Alk | 8250 | TAATACGACTCACTATAGGGAGACATCGAGACGGAGATGCTGTGGAAA/ |
| | | TAATACGACTCACTATAGGGAGACGAGGTGAATATGCCATCGAGGAAG |
| aPKC | 10261 | GCTTCTAATACGACTCACTATAGGCTCTCCTTCCACAACGAAAT/ |
| | | GCTTCTAATACGACTCACTATAGAACCACAAAAAGTATGCACAAA |
| aPKC | 10261 | TAATACGACTCACTATAGGGAGAGCAGCGCAAGCAACAACAACTA/ |
| | | TAATACGACTCACTATAGGGAGAGATGGTAAATGGCTAAACAAAACGCTCAAT |
| aur | 3068 | TAATACGACTCACTATAGGGAGAACGTGCGCATATATCTGATCTTGGA/ |
| | | TAATACGACTCACTATAGGGAGAATTAAGGACCAGCAGCTTGGAAATG |
| aur | 3068 | TAATACGACTCACTATAGGGAGAACGTGCGCATATATCTGATCTTGGA/ |
| | | TAATACGACTCACTATAGGGAGAATTAAGGACCAGCAGCTTGGAAATG |
| auxillin | 1107 | GCTTCTAATACGACTCACTATAGACACACAATTGGTCGCTCAAA/ |
| | | GCTTCTAATACGACTCACTATAGGATTAGGAGCATGTGCCTGTG |
| BABO | 8224 | TAATACGACTCACTATAGGGAGAACAATGGAACTTGGACACAGTTGTGG/ |
| | | TAATACGACTCACTATAGGGAGACATCGTAATACGGCAATTGATACTC |
| BcDNA:GH04978 | 7028 | TAATACGACTCACTATAGGGAGACTCGCAGCAGCTGGTTGTCCACACC/ |
| | | TAATACGACTCACTATAGGGAGACCGCTCTCGTCGCCTGTCTGATTCAAA |
| BcDNA:GH07910 | 2829 | TAATACGACTCACTATAGGGAGACTGCCAGTCAGCGACAACAAGAAGA/ |
| | | TAATACGACTCACTATAGGGAGATTGTTGCTGCTGTTGCTGCTGGGAT |
| BcDNA:LD09009 | 6386 | TAATACGACTCACTATAGGGAGACCAACATACTGCTGGGCCTGGAAAA/ |
| | | TAATACGACTCACTATAGGGAGAGCTGCTGGTCTGTGGCTTCATCTTA |
| BcDNA:LD22679 | 1344 | TAATACGACTCACTATAGGGAGATAAGGCCAAGACTTTCTGCTATCTT/ |
| | | TAATACGACTCACTATAGGGAGAAAATCAGCCATGCACCTTAGTGTTT |
| BcDNA:LD23371 | 8878 | TAATACGACTCACTATAGGGAGATCGACTTCGGCCTGGCGTCTAAGTT/ |
| | | TAATACGACTCACTATAGGGAGATGTCACACTTGCTGCCATCCACCAC |
| BcDNA:LD28657 | 1098 | TAATACGACTCACTATAGGGAGACAGCTGGACCACCAAAACATTGTCA/ |
| | | TAATACGACTCACTATAGGGAGATTGATGGCAGTTGACTCGCTGTTAC |
| BEST:CK01209 | 9085 | TAATACGACTCACTATAGGGAGAACCACCCACACCTCATCTCGCCCACCCACACG/ |
| | | TAATACGACTCACTATAGGGAGACGGCGGCGACAGCGACAGCGAGAGGAAGT |
| BEST:CK01209 | 9085 | TAATACGACTCACTATAGGGAGAATTGGCGAGTGACCCTGCTGGAC/ |
| | | TAATACGACTCACTATAGGGAGAGGGGGTAACGGGCTCAAAGGGTAG |
| bsk | 5680 | TAATACGACTCACTATAGGGAGATTTACACTCAGCAGGAATTATTCAC/ |
| | | TAATACGACTCACTATAGGGAGATCTGGATCAATGACTAGCATTTTAC |
| bt | 1479 | TAATACGACTCACTATAGGGAGACGGACCACTTCAAATATCAGATGTG/ |
| | | TAATACGACTCACTATAGGGAGAGGAAACTCGGAATTTGTAGGTTTGG |
| Btk29A | 18355 | TAATACGACTCACTATAGGGAGATTGGATCGGGACAGTTTGGTGTTGT/ |
| | | TAATACGACTCACTATAGGGAGATCTTAGAGGAGAAGCGCGTGTAGTT |
| btl | 6714 | TAATACGACTCACTATAGGGAGAGTGATATTATTTCGACCGCAACT/ |
| | | TAATACGACTCACTATAGGGAGAAGCAACTTCAGGGATCGACTCTCATTCAGG |
| BubR1 | 7838 | TAATACGACTCACTATAGGGAGAAAAATCCATCATGCCACGCAGAGTT/ |
| | | TAATACGACTCACTATAGGGAGAGCTGAGAGGTTTCCAATATGGTAGA |
| BubR1 | 7838 | TAATACGACTCACTATAGGGAGACCAGTAGGCGCAATGTAGGTC/ |
| | | TAATACGACTCACTATAGGGAGATTGCGATGGATAAATGGATGCT |
| Cad96Ca/HD-14 | 10239 | TAATACGACTCACTATAGGGAGACCAGCTGCCCGGTGTTCAATCCAT/ |
| | | TAATACGACTCACTATAGGGAGACAGAGGCGTCGGCGGCACAAGTA |
| Caki | 13412 | TAATACGACTCACTATAGGGAGATCTGTTACCTTCAAGATAGTTCCTT/ |
| | | TAATACGACTCACTATAGGGAGATCGATATCAAGGTGTTCTTAATGTG |
| Caki | 13412 | TAATACGACTCACTATAGGGAGACGAGGGAATGCTCTACATGGTCTTC/ |
| | | TAATACGACTCACTATAGGGAGACGTTCGCCGAGATTGATTTCCACTC |
| CaMKI | 1495 | TAATACGACTCACTATAGGGAGATGGTTACTGGTGGAGAACTCTTTGA/ |
| | | TAATACGACTCACTATAGGGAGATGCTTGCTTGCAAGTATACCTCTTC |
| CaMKII | 18069 | TAATACGACTCACTATAGGGAGAATTACATTGGCGAGCTTTACTTTAC/ |
| | | TAATACGACTCACTATAGGGAGAAACTTACTTTCCCAACTCTTCTT |
| cdc2 | 5363 | TAATACGACTCACTATAGGGAGAAAATTTCGTTGCTTAAGGAGTTGA/ |
| | | TAATACGACTCACTATAGGGAGAATCGACGGGACAGGAATACC |
| cdc2c/CDK2 | 10498 | TAATACGACTCACTATAGGGAGAAAGGATTGTTTACGAGGTTATGAC/ |
| | | TAATACGACTCACTATAGGGAGATTGTTGCCGGAAATGACTACG |
| cdc2rk | 1362 | TAATACGACTCACTATAGGGAGAAGGATGTCCAGCTTAAAGTCAAACGAT/ |
| | | TAATACGATCACTATAGGGAGACCCACCACCACCTCACGCAAGCGGACAATA |
| cdi | 6027 | TAATACGACTCACTATAGGGAGATCTCTATTACGAAGCAAGCAAAAAGTGATT/ |
| | | TAATACGACTCACTATAGGGAGACCAGATGAAAGGGGAGGCGGCCGATGAGTC |
| Cdk4 | 5072 | TAATACGACTCACTATAGGGAGACGAACAAGGCAAATAAATCAAGTCAC/ |
| | | TAATACGACTCACTATAGGGAGAGGGGGCAAATCGTCGGAATA |
| Cdk5 | 8203 | TAATACGACTCACTATAGGGAGACACTGCGACCAGGACCTCAAGAAGT/ |
| | | TAATACGACTCACTATAGGGAGACCACGAGGTGATGGCCGGAAAAGAT |
| Cdk7 | 3319 | TAATACGACTCACTATAGGGAGATGGTGGACGTTTTCGGTCAACTTTC/ |
| | | TAATACGACTCACTATAGGGACAGTTTGCTCAATGCGGCCATTCA |
| Cdk8 | 10572 | TAATACGACTCACTATAGGGAGACTTTCCGCAGGACAAGGACTGGGAG/ |
| | | TAATACGACTCACTATAGGGAGATGCTGTTGCTGCTGCTGCTGGTGGT |
| Cdk9 | 5179 | TAATACGACTCACTATAGGGAGAAGCTGCCAACGTGCTGATTACCAAG/ |
| | | TAATACGACTCACTATAGGGAGATGGGCATGGGATCCGTCCAGAAGAA |
| CG10177 | 10177 | TAATACGACTCACTATAGGGAGAATGGTGGACTCTTGTAAACGTTGGG/ |
| | | TAATACGACTCACTATAGGGAGAGGATGAAGAGCAAACCAGCAGATTC |
| CG10522 | 10522 | TAATACGACTCACTATAGGGAGAAGGAGACGGAATTGCGCCAGAAACT/ |
| | | TAATACGACTCACTATAGGGAGAAGCTCTCTCTTGGGTGGCAAACAGT |
| CG10522 | 10522 | TAATACGACTCACTATAGGGAGACGCCGTTCTGGAAATGCAAGGAGTG/ |
| | | TAATACGACTCACTATAGGGAGAGACGTTTCCAGCACAGGCTTGTGAC |
| CG10673 | 10673 | TAATACGACTCACTATAGGGAGAAAAGGAGAAGCCTGCCTGATCAAGG/ |
| | | TAATACGACTCACTATAGGGAGACGTGCTCGAAGATATACGGCTGTTC |
| CG10738 | 10738 | TAATACGACTCACTATAGGGAGAGATTTCAAAACCATCCGCACCAAAC/ |
| | | TAATACGACTCACTATAGGGAGAGGTGACGGATCTTAAACTCTGATAC |
| CG10951 | 10951 | TAATACGACTCACTATAGGGAGAATACGCAGCGGAAGCAATCGACTTG/ |
| | | TAATACGACTCACTATAGGGAGAAGTTGATGGCACTGGAGATCTGTTC |
| CG10967 | 10967 | TAATACGACTCACTATAGGGAGAAGCGCAAGAGCAGTGTGAGCAGTGA/ |
| | | TAATACGACTCACTATAGGGAGACGCCAGCACAAAGTTCAGCTTGGAC |
| CCG11221 | 11221 | TAATACGACTCACTATAGGGAGAACGTGGAGCTGCCGCTGATGACCTT/ |
| | | TAATACGACTCACTATAGGGAGAGCTTTCACCTTGTGCACCAGCAGACC |
| CG11228 | 11228 | TAATACGACTCACTATAGGGAGATGGCTACGACTGTGTGGCAGACATA/ |
| | | TAATACGACTCACTATAGGGAGACACCTTGCTCTGCATGCGCTATCAT |
| CG11228 | 11228 | TAATACGACTCACTATAGGGAGAGGCAACCCAGGAAAACGGAATG/ |
| | | TAATACGACTCACTATAGGGAGAGGAGCAGCTGCCTTGGACA |
| CG11533 | 11533 | TAATACGACTCACTATAGGGAGAAAATCAATCATGCCAGCCTTCTTC/ |
| | | TAATACGACTCACTATAGGGAGACATCTTGACGGACTCCTTAGCCTTGAC |
| CG11660 | 11660 | TAATACGACTCACTATAGGGAGATTTCTACGGGCAAAGAGGCTAATGT/ |
| | | TAATACGACTCACTATAGGGAGAAGGAAGTCAAAGGAATGCGGATGAT |
| CG11859 | 11859 | TAATACGACTCACTATAGGGAGATGGATCAGGCAGATGAGGAGGATGA/ |
| | | TAATACGACTCACTATAGGGAGAAGACGGAGCTATTGTGCTGGTTGTG |
| CG11870 | 11870 | TAATACGACTCACTATAGGGAGAGGAAATTGTGGAAGGCACACCATAC/ |
| | | TAATACGACTCACTATAGGGAGAAAGCAGGAGAATGGACATCGACTA |
| CG12069 | 12069 | TAATACGACTCACTATAGGGAGACACGGTCAACCTGATAGCCTCCTAC/ |
| | | TAATACGACTCACTATAGGGAGACACCAAATGACGCAGAGCTCCACTG |
| CG12147 | 12147 | TAATACGACTCACTATAGGGAGATCATGAGAAGGACCAGCGACAAGAT/ |
| | | TAATACGACTCACTATAGGGAGACCGAAGTCAATCATGTACACCTGAG |
| CG1227 | 1227 | TAATACGACTCACTATAGGGAGACAGTCTAATCGACCTTGGGGAAAAC/ |
| | | TAATACGACTCACTATAGGGAGAATAACTCTGGAGCCCGGTAGACAAT |
| CG14030 | 14030 | TAATACGACTCACTATAGGGAGAAGGAGCATAGCGGACCGTACACAAA/ |
| | | TAATACGACTCACTATAGGGAGAGAGTTATCCAAAGCTGTGGCACTGG |
| CG14163 | 14163 | TAATACGACTCACTATAGGGAGAAGTGGCAGCGCTGTGGGAATAACG/ |
| | | TAATACGACTCACTATAGGGAGACACCCGATGCTGCACTGATGGACA |
| CG14217 | 14217 | TAATACGACTCACTATAGGGAGAGCTGCGGCACAGAATCATCACCATA/ |
| | | TAATACGACTCACTATAGGGAGATGACTGCATTGGAGATGGCCTGTTG |
| CG14305 | 14305 | TAATACGACTCACTATAGGGAGATCACAAGATCGGCGAGGGGTCTTAT/ |
| | | TAATACGACTCACTATAGGGAGAAAGCAGCTGATCCGCAGTATGTCTC |
| CG15072 | 15072 | TAATACGACTCACTATAGGGAGAGCAGTCAGAGATGCAGGAGCAGGAA/ |
| | | TAATACGACTCACTATAGGGAGAATCGCGCCAGGCATTCCAGCTCAAA |
| CG17090 | 17090 | TAATACGACTCACTATAGGGAGAGGCTCAAAGCGAATGTGGCTACCAA/ |
| | | TAATACGACTCACTATAGGGAGACCTGGTGATGATGATGTCCATGACTG |
| CG17309 | 17309 | TAATACGACTCACTATAGGGAGACAGACGACGAACCAGCAGCAACAAC/ |
| | | TAATACGACTCACTATAGGGAGAGGGAAGCATGGTTCATGGCAGTGGT |
| CG17528 | 17528 | TAATACGACTCACTATAGGGAGATGCGACATCCCCTCAGAGCGTTCCT/ |
| | | TAATACGACTCACTATAGGGAGAATTTGATTCTTAGTGCCTTTCTTGTCG |
| CG1760 | 1760 | TAATACGACTCACTATAGGGAGACGTCTGTCAAGGCTCTGGTGAAGAA/ |
| | | TAATACGACTCACTATAGGGAGATGATCCAGGCTCAAGTTGTTGGTGG |
| CG17698 | 17698 | TAATACGACTCACTATAGGGAGACAACATCACCTCTAGATCGAGTTTA/ |
| | | TAATACGACTCACTATAGGGAGATGAATCAGCTAGAAACGGCACATTT |
| CG1776 | 1776 | TAATACGACTCACTATAGGGAGACCAGAGCCAAAGCACCTACGATGAC/ |
| | | TAATACGACTCACTATAGGGAGAATGCAGCAGGCAATCCTTGGTGGTG |
| CG18020 | 18020 | TAATACGACTCACTATAGGGAGATGTACGAGGTGATTGCTCAGAATCC/ |
| | | TAATACGACTCACTATAGGGAGAATGGGCTTGTCCTGGAAGTACCAT |
| CG1951 | 1951 | TAATACGACTCACTATAGGGAGACGGGGTTAAGACACTTAGCTATTTG/ |
| | | TAATACGACTCACTATAGGGAGACTCTGCAGACACAGTTTCTTGATTC |
| CG1973 | 1973 | TAATACGACTCACTATAGGGAGAGCTGGATCTGTTCATCGCGCACTTG/ |
| | | TAATACGACTCACTATAGGGAGACAAACTGGGATCCTCGGAGACCTTC |
| CG2049 | 2049 | TAATACGACTCACTATAGGGAGAGTTATACCACAGTTGGGGAAGCTTTAC/ |
| | | TAATACGACTCACTATAGGGAGATTCTTCAGTGCCTTAATAGCGTAGTA |
| CG2309 | 2309 | TAATACGACTCACTATAGGGAGAAAGAGCTGGACCAAACTGTGGAAAG/ |
| | | TAATACGACTCACTATAGGGAGATCATCATAGATGCGTCTCGAGGAGA |
| CG2577 | 2577 | TAATACGACTCACTATAGGGAGAGCGCAAGATCGGCTGTGGATCCTTC/ |
| | | TAATACGATCATATAGGGAGACCAATGGAGGCGTACCTGGCTGTTC |
| CG2905 | 2905 | TAATACGACTCACTATAGGGAGAGATAAAGTTCTTGCTACAGTGGAAA/ |
| | | TAATACGACTCACTATAGGGAGAAAAGGTAAAGCATTTGAATCAGGAG |
| CG3105 | 3105 | TAATACGACTCACTATAGGGAGATGTGACGCTTTACGTTCTGATGTTT/ |
| | | TAATACGACTCACTATAGGGAGATCGCGTAAAGAGTGTCCATTTTGTT |
| CG3216 | 3216 | TAATACGACTCACTATAGGGAGATCTACCAAATCCTGCCGCGTCCTGT/ |
| | | TAATACGACTCACTATAGGGAGAGGTGGCCGAGGACACATGTATCTTG |
| CG3277 | 3277 | TAATACGACTGACTATAGGGAGAGATTGTGCTGATTCTCCTGCTGGTGCTA/ |
| | | TAATACGACTCACTATAGGGAGAAAGAACATTGTACGAAGTGCCTAAAAG |
| CG3608 | 3608 | TAATACGACTCACTATAGGGAGATCGCTTGGGAGGTGGATTTGAAC/ |
| | | TAATACGACTCACTATAGGGAGAGCGTGCGCAGCGTGGACTC |
| CG4041 | 4041 | TAATACGACTCACTATAGGGAGAGGTCGCTGGCCCTGGTAATGGTGGAG/ |
| | | TAATACGACTCACTATAGGGAGAGCGGCGAGTGGAGCAGGGGAAAGTAGA |
| CG4224 | 4224 | TAATACGACTCACTATAGGGAGAGGAGGATCGGTTGAAGCTAAGGATA/ |
| | | TAATACGACTCACTATAGGGAGAGAACTGGAGCTGATCTTGCGTTTCA |
| CG4523 | 4523 | TAATACGACTCACTATAGGGAGAAAACATCAACAGCTCTGTGGACAGT/ |
| | | TAATACGACTCACTATAGGGAGACTGCAGCTCGATTAGCACATTATCA |
| CG4527 | 4527 | TAATACGACTCACTATAGGGAGAATAATACGGCATCTGGCAGTCATAG/ |
| | | TAATACGACTCACTATAGGGAGATCCTTGGTAAGACCTTGAGCATTTG |
| CG4549 | 4549 | TAATACGACTCACTATAGGGAGAGGTCACACCAGTCTTTGCGCTCTAC/ |
| | | TAATACGACTCACTATAGGGAGACCATGGCCTGCACTAGATTCTGGGT |
| CG4588 | 4588 | TAATACGACTCACTATAGGGAGAAAAATGGTTTGGATCTGCTGGAGGA/ |
| | | TAATACGACTCACTATAGGGAGACAATCCGAAGAGCTGCGAAATGTTG |
| CG4629 | 4629 | TAATACGACTCACTATAGGGAGAGTGCCGATCTGATGCAGTGGGAGAT/ |
| | | TAATACGACTCACTATAGGGAGATAGCTGGCTCAGATCCTCGGTGTTC |
| CG4839 | 4839 | TAATACGACTCACTATAGGGAGACGGGATGCAAAGGACACTGGAGATG/ |
| | | TAATACGACTCACTATAGGGAGACAACGGCGGTGCTGGCGGTATGTTA |
| CG4945 | 4945 | TAATACGACTCACTATAGGGAGAGAGCGCAAAGGAGATTAACAGCACCCT/ |
| | | TAATACGACTCACTATAGGGAGATCATCGCCGAAAGAAGGAGCTCTTG |
| CG5169 | 5169 | TAATACGACTCACTATAGGGAGAAGAAGCTGATGCAGACCACACACTC/ |
| | | TAATACGACTCACTATAGGGAGAGCGCGGCGTTATTTGCATGGAATGA |
| CG5483 | 5483 | TAATACGACTCACTATAGGGAGATGAAGTCTTTCTACAAGACAACCAG/ |
| | | TAATACGACTCACTATAGGGAGAGCAGACTGTGGTATGACATATTCAG |
| CG5790 | 5790 | TAATACGACTCACTATAGGGAGACGATTTCGGATTGGCTCAAAGGATA/ |
| | | TAATACGACTCACTATAGGGAGAGACCAGAGAGCAAAGAGAGCATTAT |
| CG6114 | 6114 | TAATACGACTCACTATAGGGAGAGGGTCACAGCTGGCGGCAAAGGGGA/ |
| | | TAATACGACTCACTATAGGGAGACGGAGAGTTGCAGCGCGTGGGACTG |
| CG6498/MAST | 6498 | GCTTCTAATACGACTCACTATAGGTGTACGGCACACCCGAGTA/ |
| | | GCTTCTAATACGACTCACTATAGAGACGATCCCGTGGATTCTG |
| CG6535 | 6535 | TAATACGACTCACTATAGGGAGACAATCTGAAGATGGGCAACCAACAA/ |
| | | TAATACGACTCACTATAGGGAGATCTGATTTAGCTGCTGCTCATCCAA |
| CG6800 | 6800 | GCTTCTAATACGACTCACTATAGTCCTGACCTAACTGGTCTCTCC/ |
| | | GCTTCTAATACGACTCACTATAGCATATCCACTCCGGTTCCATA |
| CG7094 | 7094 | TAATACGACTCACTATAGGGAGAACCAGCTGCTAATGCGAATTGAGTG/ |
| | | TAATACGACTCACTATAGGGAGAGCGGAAAAGTATGCGGAATATCTGG |
| CG7097 | 7097 | TAATACGACTCACTATAGGGAGACACATCAGGCGGCACAGCAGGAACA/ |
| | | TAATACGACTCACTATAGGGAGAAGCTGATGACGCAGAGGACGAGATG |
| CG7125 | 7125 | GCTTCTAATACGACTCACTATAGCCTCATCTGGACACGTAGAGC/ |
| | | GCTTCTAATACGACTCACTATAGGGAGCTCCTGTCCTGTTCTG |
| CG7156 | 7156 | TAATACGACTCACTATAGGGAGAAGAGATTCGATGCGGCAGTCATCCA/ |
| | | TAATACGACTCACTATAGGGAGATGACTGAACTCTAGAGCCGCCTCAT |
| CG7177 | 7177 | TAATACGACTCACTATAGGGAGAGACGAAGATATCGGTATACGAGTGG/ |
| | | TAATACGACTCACTATAGGGAGATGGATAGAGCCAGGCTTGTTTCTGA |
| CG7236 | 7236 | TAATACGACTCACTATAGGGAGACTGACCAAACAGATCTGCTACCAGA/ |
| | | TAATACGACTCACTATAGGGAGATGTCCAGGCACTTCTTGAGAAAGTC |
| CG7597 | 7597 | TAATACGACTCACTATAGGGAGAGGCAGAAGGCGCTGAAGGAAATCAT/ |
| | | TAATACGACTCACTATAGGGAGAGGAAGATTGAGCACGCTCTTGTTGG |
| CG7616 | 7616 | TAATACGACTCACTATAGGGAGAGGCCTGGCTTACGATGGGATAGTAA/ |
| | | TAATACGACTCACTATAGGGAGAGTGCCTAGGTCGCTGATGAATCTCT |
| CG7643 | 7643 | TAATACGACTCACTATAGGGAGAAAGCCGGATGCAGACTTCATTACCC/ |
| | | TAATACGACTCACTATAGGGAGAACCACCTTCAGGGCGAACTCATTTC |
| CG7643 | 7643 | TAATACGACTCACTATAGGGAGATAACAAACAGCAACAGCAACATAAC/ |
| | | TAATACGACTCACTATAGGGAGACGTCTTCGAGGTGGAGGGTAA |
| CG8173 | 8173 | TAATACGACTCACTATAGGGAGACGCACCGGAGGTCATAGACGAAGTG/ |
| | | TAATACGACTCACTATAGGGAGATGGCCGCTGGACGATCCTCGCTGAGA |
| CG8485 | 8485 | TAATACGACTCACTATAGGGAGAAACAGGAAATTCCACGAATAGAAGG/ |
| | | TAATACGACTCACTATAGGGAGAAGCATTTAGAGCCGGTAACGTGTAT |
| CG8565 | 8565 | TAATACGACTCACTATAGGGAGACCAGCATGCCGTTCGAAATGAAACA/ |
| | | TAATACGACTCACTATAGGGAGACACCTGCTGGGCAATTTGCTTGATA |
| CG8655 | 8655 | TAATACGACTCACTATAGGGAGAAAATTGCGCTGGATGCTGGTTTGGG/ |
| | | TAATACGACTCACTATAGGGAGACAGTGGTCTGATCTGGGTACTTGAG |
| CG8726 | 8726 | TAATACGACTCACTATAGGGAGAACGTGGTCGCTGGGTGGAAGTATGG/ |
| | | TAATACGACTCACTATAGGGAGAATGAAAAATGGCCGGTAAAACGCTGGAACG |
| CG8767 | 8767 | TAATACGACTCACTATAGGGAGATGCAATTCCTCGAAGATCAAAGTGAA/ |
| | | TAATACGACTCACTATAGGGAGAGGACTATCAAAGTGGAGTGCTAATC |
| CG8789 | 8789 | TAATACGACTCACTATAGGGAGAAGAACCGAAAGGTGCAGCTGGTGGA/ |
| | | TAATACGACTCACTATAGGGAGACTCTCACGCAATTCAAGAGGAGAGG |
| CG8866 | 8866 | TAATACGACTCACTATAGGGAGACTCTGGAGCATCGGGGTCATCCTCT/ |
| | | TAATACGACTCACTATAGGGAGAGCCTGCCGTTGACGTTCAGCCAACA |
| CG9222 | 9222 | TAATACGACTCACTATAGGGAGAATTCTTGAGGAGCATGGCATCATAC/ |
| | | TAATACGACTCACTATAGGGAGAGAAGGTTTTCGAGAGTATCACTTGG |
| GG9374 | 9374 | TAATACGACTCACTATAGGGAGAGACGCTGGACATGGGTAATATGTTC/ |
| | | TAATACGACTCACTATAGGGAGATTTGATCCAGGGAGAGCAGCAGGTT |
| CG9374 | 9374 | TAATACGACTCACTATAGGGAGAGTCAAGGCAGCACACCATCATCAT/ |
| | | TAATACGACTCACTATAGGGAGATGCAGCCCGCCGACACAGTA |
| CG9746 | 9746 | TAATACGACTCACTATAGGGAGAAAACAGAAGATCTGCCACGGGGACA/ |
| | | TAATACGACTCACTATAGGGAGATCCAAGTAATCCTCGGCGCTCTTTC |
| C69783 | 9783 | TAATACGACTCACTATAGGGAGAACAACCACTACAAATGCCTCAGTCC/ |
| | | TAATACGACTCACTATAGGGAGATGATGGCGGACTGCGGTTTAGATTG |
| CG9962 | 9662 | TAATACGACTCACTATAGGGAGAATCTACGAGGCCAAGCACATGGGGT/ |
| | | TAATACGACTCACTATAGGGAGACCGCCGGGACTGCACTTTACAACAA |
| CkIalpha | 2028 | TAATACGACTCACTATAGGGAGACGTCACCATGGCAAGGAAAAGAACT/ |
| | | TAATACGACTCACTATAGGGAGAGCGTGGACATCTTCTTTTCGGAGAT |
| CkIIalpha | 17520 | TAATACGACTCACTATAGGGAGACAATCAAGACGATTATCAGTTGGTC/ |
| | | TAATACGACTCACTATAGGGAGACCAGTAATTCGGGACCTTTAAAGTA |
| CKIIalpha | 17520 | TAATACGACTCACTATAGGGAGAATTAGGCCGTGGAAAGTATT/ |
| | | TAATACGACTCACTATAGGGAGACGAAGCCACACGAACATTAT |
| dco | 2048 | TAATACGACTCACTATAGGGAGACGGATAACTTCCTCATGGGTCTTGG/ |
| | | TAATACGACTCACTATAGGGAGAAGGTCCGCCAAACTTAAGCAGGTTC |
| Ddr | 11573 | TAATACGACTCACTATAGGGAGACCGGACATTGTGTGCCAGGACTATG/ |
| | | TAATACGACTCACTATAGGGAGACGCACAAATGCAGCTCACCAAATAC |
| Ddr | 9488 | TAATACGACTCACTATAGGGAGAACCACCGACACCAAACATACATAC/ |
| | | TAATACGACTCACTATAGGGAGAAATTGCCTTTTCCACACCATAGTT |
| Ddr | 9490 | TAATACGACTCACTATAGGGAGAGAATTTCACACTAAGCCATACAAG/ |
| | | TAATACGACTCACTATAGGGAGACTCTCCCAAGCCATCCAG |
| dnt | 17559 | TAATACGACTCACTATAGGGAGAGACCGGCGATCAATGTGTCACACAG/ |
| | | TAATACGACTCACTATAGGGAGAACTGGAACTTTCCGTGGCAAGGAGG |
| Doa | 1658 | TAATACGACTCACTATAGGGAGAGGCAGCACAAATACCGCTACAGGGA/ |
| | | TAATACGACTCACTATAGGGAGATTGGTCCAGCGGGTATGGCTCATAG |
| drl | 10758 | TAATACGACTCACTATAGGGAGACACGAGGAGTACGACGACGATGACT/ |
| | | TAATACGACTCACTATAGGGAGATCAGCTCTTGGAGACGGCGGTTGAA |
| Drl-2 | 3915 | TAATACGACTCACTATAGGGAGACGGGAATCGAGCACAGCATTGAGTA/ |
| | | TAATACGACTCACTATAGGGAGACTTCGTCCTGTGCTTACACTTCCAC |
| Drl-2 | 12463 | GCTTCTAATACGACTCACTATAGCCTTGACGAAGAGTCCTATGTG/ |
| | | GCTTCTAATACGACTCACTATAGCCAAGTAATTGGTAAGCTCGAA |
| Dsor1 | 15793 | TAATACGACTCACTATAGGGAGAGCTGTCCGACGAGGATCTGGAGAAG/ |
| | | TAATACGACTCACTATAGGGAGAAGCTACGGGTGCCCACAAAGGAGTT |
| EG:22E5.8 | 4290 | TAATACGACTCACTATAGGGAGATCGCTGTTCCATTCAGGCCACCAAG/ |
| | | TAATACGACTCACTATAGGGAGAAGGCACCTGGTCCGATTGGCTGAT |
| Egfr | 10079 | TAATACGACTCACTATAGGGAGAGGCCATTAAGGAGCTGCTCAAGTCC/ |
| | | TAATACGACTCACTATAGGGAGACTGGCCAAAGGTCAGCAGTTCCCAA |
| Eip63E | 10579 | TAATACGACTCACTATAGGGAGACTACAATTCGGAGGAATACTTGGAC/ |
| | | TAATACGACTCACTATAGGGAGATGACGATGTTGCTGTGTTTCAGTTC |
| Eph | 1511 | TAATACGACTCACTATAGGGAGAGGTAACGACATACACTGTGCAGATA/ |
| | | TAATACGACTCACTATAGGAGACTGAACCAACGGATTGAAGAGTTTG |
| Fak56D | 10023 | TAATACGACTCACTATAGGGAGATCATCCACGTGCATATGCCGAACAA/ |
| | | TAATACGACTCACTATAGGGAGAGAAATAATGACGAATGCCCAGACAG |
| for | 10033 | TAATACGACTCACTATAGGGAGACGTTCAGCAGAAGTGTGGTCAGGTC/ |
| | | TAATACGACTCACTATAGGGAGACCGTCCGCTGGCAGTTGTACAGGAT |
| for | 10033 | TAATACGACTCACTATAGGGAGAGAGGAGCAGAGACAGATACACACAC/ |
| | | TAATACGACTCACTATAGGGAGAAAGGCTTCGGGGATCCTGGTTCAAT |
| Fps85D | 8874 | TAATACGACTCACTATAGGGAGACAATAGCAATCACAGTGCCTCACAG/ |
| | | TAATACGACTCACTATAGGGAGAGCACGCAATAGCAGTGATCCTTCAT |
| Fps85D | 8874 | TAATACGACTCACTATAGGGAGATACAAGGCCAAACTGAAGTCCACCA/ |
| | | TAATACGACTCACTATAGGGAGACATCAGTATGCCATAGGACCACACA |
| fray | 7693 | TAATACGACTCACTATAGGGAGAATTAAGCGCATCAACCTGGAGAAGT/ |
| | | TAATACGACTCACTATAGGGAGACCAAATGTCCGCCTTAAAGTCATAG |
| fray | 7693 | TAATACGACTCACTATAGGGAGAACCCGCCAATCTGTCTAGCAATAATGT/ |
| | | TAATACGACTCACTATAGGGAGACTTCCTTCAGTACCGTGGCAATGG |
| fs(1)h | 2252 | TAATACGACTCACTATAGGGAGACGGGGCTGACGGACAATTTCTTGAT/ |
| | | TAATACGACTCACTATAGGGAGACTGTTGGTGGTGTTGCTGCTGATGT |
| fu | 6551 | TAATACGACTCACTATAGGGAGAGCATATCCTGGACGCAGCTGTTGTG/ |
| | | TAATACGACTCACTATAGGGAGAACTGGCGTACGGTTGGAGCGACTAT |
| Gcn2 | 1609 | TAATACGACTCACTATAGGGAGAAGAGCGACGAGGTGCTGGAACACAC/ |
| | | TAATACGACTCACTATAGGGAGATCGCGTAATCGGGGCAGTTCACTGG |
| gek | 4012 | TAATACGACTCACTATAGGGAGAGCAACAAACACAGGAAAGGCTGAAG/ |
| | | TAATACGACTCACTATAGGGAGAGGATATGAGGTCCGATCTGGTTTGA |
| gish | 6963 | GCTTCTAATACGACTCACTATAGGCATATACCATATCGGGAGCAT/ |
| | | GCTTCTAATACGACTCACTATAGCTCGTTTCGTATCACCGATTTT |
| gish | 6963 | TAATACGACTCACTATAGGGAGAACTTGTTACTAATCGATTTCCGTTCTCTTTC/ |
| | | TAATACGACTCACTATAGGGAGACGTAGTGCGTTCTGGATCCTCTGTTATTT |
| Gprk2 | 17998 | GCTTGTAATACGACTCACTATAGAGCTCTCAAACTCCCGGAAC/ |
| | | GCTTCTAATACGACTCACTATAGCAGCGACATCAATCACAAGAA |
| grp | 17161 | TAATACGACTCACTATAGGGAGACAGCGACGATGACTTCAATGTCAGA/ |
| | | TAATACGACTCACTATAGGGAGATGGGTCCTTTAAGCACGATATCCTC |
| GSK3b | 31003 | TAATACGACTCACTATAGGGAGAAACCACTTCGCAGCAGAGA |
| | | TAATACGACTCACTATAGGGAGAATCCCGATGGTGAAGGTGT |
| gwl | 7719 | TAATACGACTCACTATAGGGAGAGAAGCTTACTGATTTTGGGTTGAG/ |
| | | TAATACGACTCACTATAGGGAGACGTTTGTAGTGCAGGTATGAGTAA |
| gwl | 7719 | TAATACGACTCACTATAGGGAGAGCGATAGCAAGATATCTGGTGTTTC/ |
| | | TAATACGACTCACTATAGGGAGAGCTTAGGTTGTCCACATTCTTCTCA |
| Gyc32E | 6275 | TAATACGACTCACTATAGGGAGAGCGTGATTATGCGTCCAACGTGATA/ |
| | | TAATACGACTCACTATAGGGAGACTGTCAGCCAGCATCTGAAGATTGA |
| Gyc76C | 8742 | TAATACGACTCACTATAGGGAGAACAGGCCTCGCTTAGCACGCTGAAT/ |
| | | TAATACGACTCACTATAGGGAGAGGAAAGCTGCCTGAATAGCGGAGAG |
| hep | 4353 | TAATACGACTCACTATAGGGAGAGAGCTGATGTCCATGTGCTTTGACA/ |
| | | TAATACGACTCACTATAGGGAGACTGATGGTTCTTTGTGAGGCACTTG |
| hop | 1594 | TAATACGACTCACTATAGGGAGAAGATTCGCTATCCGGAGGACAAGGA/ |
| | | TAATACGACTCACTATAGGGAGACGTGTGGAAAGACTCGCACATAGAC |
| htl | 7223 | TAATACGACTCACTATAGGGAGATCTGCGAGTGGTGCGAAGTCTTCAC/ |
| | | TAATACGACTCACTATAGGGAGACCGCACCAAGCTGGCAATGTCATCA |
| ia1 | 6620 | TAATACGACTCACTATAGGGAGATGTTCAAAGAGGAGCTGCGCAAGGG/ |
| | | TAATACGACTCACTATAGGGAGACTCCATGCGCCGGATCTTGCTGTAG |
| ik2 | 2615 | TAATACGACTCACTATAGGGAGAGGGCAAACCATATACAAGCTTACTG/ |
| | | TAATACGACTCACTATAGGGAGAAACTAGTCCGATTGGTGAAGAACAC |
| Ilk | 10504 | TAATACGACTCACTATAGGGAGAGATAAAAGAGCGCAGCGATGTGAAT/ |
| | | TAATACGACTCACTATAGGGAGAGGCGAATTGCATGCTCCAATAATAG |
| inaC | 6518 | TAATACGACTCACTATAGGGAGATCAGTGCGAGGGTAGGTAGAAATGTT/ |
| | | TAATACGACTCACTATAGGGAGAGTCCGCACTCGTCGCAGAATGT |
| InR | 18402 | TAATACGACTCACTATAGGGAGAACTCCTGATGGGCAGACTGTAATGG/ |
| | | TAATACGACTCACTATAGGGAGACACTGGGTGACTTGTCAAGTTGGTG |
| ird5 | 4201 | GCTTCTAATACGACTCACTATAGTCTGCAATGGTGCGATAATTT/ |
| | | GCTTCTAATACGACTCACTATAGCCTTTCTGCCTCTTGGATAGC |
| ire-1 | 4583 | GCTTCTAATACGACTCACTATAGTCAGGAGAATGTTCAGGTTCC/ |
| | | GCTTCTAATACGACTCACTATAGATATTCCTTGGCCAGCTCAG |
| JIL1 | 6297 | GCTTCTAATACGACTCACTATAGTCGAGTGTAACGGAAATCGAC/ |
| | | GCTTCTAATACGACTCACTATAGGAATCGGAGTGTGTGTGTGTG |
| KP78a | 6715 | TAATACGACTCACTATAGGGAGAATGCCAGGGTGATCTTCCGACAGTT/ |
| | | TAATACGACTCACTATAGGGAGAAAACGGACGCAATCGGTCGGATTCA |
| KP7Bb | 17216 | TAATACGACTCACTATAGGGAGATTGGTGTCTGCTATTGAATACTGTC/ |
| | | TAATACGACTCACTATAGGGAGACGATTTACATCATGCAGATCCATAG |
| ksr | 2899 | TAATACGACTCACTATAGGGAGAATGGGCTACCTGCACGCAAGGGAGA/ |
| | | TAATACGACTCACTATAGGGAGAAAAGGTTGACGGGGTGGGAGGGACT |
| lic | 12244 | TAATACGACTCACTATAGGGAGAGGACCTTTGACATCGATGCAGATAG/ |
| | | TAATACGACTCACTATAGGGAGAGGCATCAATGGTTTTGGCAATGGAG |
| LIMK1 | 1848 | GCTTCTAATACGACTCACTATAGCATTGTCGGGGTCAACTACTG/ |
| | | GCTTCTAATACGACTCACTATAGGTCCTTGGGTATCTCGACCAG |
| Lk6 | 17342 | TAATACGACTCACTATAGGGAGAAAGATGATGAGGATGGAGAGAATGA/ |
| | | TAATACGACTCACTATAGGGAGAGTGTTGTAGTCATAACTGGTGTTTG |
| lok | 10895 | TAATACGACTCACTATAGGGAGACTGCGAACTTACCAATCCAGTTTAT/ |
| | | TAATACGACTCACTATAGGGAGATCGCTAAGTAGTTTGTTGCTGATGA |
| MAPk-Ak2 | 3086 | TAATACGACTCACTATAGGGAGACCACTGACGGACGACTACGTGACCT/ |
| | | TAATACGACTCACTATAGGGAGAGCAGCGTGTAACTGGTGAATGTCTC |
| mbt | 18582 | TAATACGACTCACTATAGGGAGAGCCAGATCCAATTCGCTGCGGAGTT/ |
| | | TAATACGACTCACTATAGGGAGATTGGTGATGGTGCGGATGCGGATGA |
| mbt | 18582 | TAATACGACTCACTATAGGGAGACCGGTGCCCATCCCTCCCTGCTCTAT/ |
| | | TAATACGACTCACTATAGGGAGACCCGCTGGAACTGGATGCCCTGGAC |
| mei-41 | 4252 | TAATACGACTCACTATAGGGAGATTTTGAGAAGCCATTGAAGGAGGAG/ |
| | | TAATACGACTCACTATAGGGAGAGTACCTGGAGACATTCATCGGTTAT |
| Mekk1 | 7717 | TAATACGACTCACTATAGGGAGATTACCTGCGTGCCAAGTTCG/ |
| | | TAATACGACTCACTATAGGGAGAGCTCTGCTCGCGTCGTAATCGTATG |
| Mekk1 | 7717 | GCTTCTAATACGACTCACTATAGCATTTTCAGTGTCGAGCCATT/ |
| | | GCTTCTAATACGACTCACTATAGCACCGTAGCATCGTAGTGGTT |
| Mkk4 | 9738 | TAATACGACTCACTATAGGGAGAACTTCAGACGATCTCGAGGATGAGG/ |
| | | TAATACGACTCACTATAGGGAGACGCATCCTTGGTCTTGGCAATAGAG |
| mnb | 7826 | TAATACGACTCACTATAGGGAGAGCACCAACAGCCTGGGCAGCCTGAA/ |
| | | TAATACGACTCACTATAGGGAGATCCGGCGGGCTGAGATGGGAAGAGA |
| mnb | 7826 | TAATACGACTCACTATAGGGAGATAGCGGCGGCGTTATGGAT/ |
| | | TAATACGACTCACTATAGGGAGAGCTGGGCACGACGTTTCTTTTT |
| Mpk2 | 5475 | TAATACGACTCACTATAGGGAGATCAAACATTGCCGTCAACGAGGATT/ |
| | | TAATACGACTCACTATAGGGAGACAAATCCATATCCTCGAAGCTGTGA |
| msn | 16973 | TAATACGACTCACTATAGGGAGAGCTCTGCTCGCGTCGTAATCGTATG/ |
| | | TAATACGACTCACTATAGGGAGATGCTACCACCGCTTCCGCTACCACTA |
| MYT1 | 10569 | GCTTCTAATACGACTCACTATAGCACGACGACAAACACAGACAC/ |
| | | GCTTCTAATACGACTCACTATAGTTGCATGTACAGTCGGTCGTA |
| Nak | 10637 | TAATACGACTCACTATAGGGAGACACAGAACACGCAATCAGGGGAAAC/ |
| | | TAATACGACTCACTATAGGGAGAAGATGGGTAACGACCGCTGGTATTG |
| Nek2 | 17256 | GCTTCTAATACGACTCACTATAGACTACATGAGTCCGGAGTTGGT/ |
| | | GCTTCTAATACGACTCACTATAGTTCACTTCGCAAATCTGGAGTA |
| ninaC | 54125 | TAATACGACTCACTATAGGGAGAAGCTACTCGGGCAAGTCCACAAATG/ |
| | | TAATACGACTCACTATAGGGAGACAGCCAAAACTTTGCGAACGGTCTC |
| nmo | 7892 | TAATACGACTCACTATAGGGAGAGCCGACCACATCAAGGTGTTCCTGT/ |
| | | TAATACGACTCACTATAGGGAGAAGACGAGCATCTGGCAGAGCAAGTG |
| Nrk | 4007 | TAATACGACTCACTATAGGGAGAAGATCTACTAGTCGCTGTTAAGATG/ |
| | | TAATACGACTCACTATAGGGAGAAAGCGAGAACTTGTTGTACAGTATG |
| otk | 8967 | TAATACGACTCACTATAGGGAGACAAGCCGACAATTCAGTGGGACAAG/ |
| | | TAATACGACTCACTATAGGGAGACTGCAGGCTGTGTCATCGGATTTCT |
| p38b | 7393 | TAATACGACTCACTATAGGGAGAGCGCAAAATGGCCAAATTCTACAAG/ |
| | | TAATACGACTCACTATAGGGAGAAAATCCAGGATGCGAAGCTCACAGT |
| P38c | FBgn0046322 | TAATACGACTCACTATAGGGAGATGAGACTACGAGGCACTGAAAAT/ |
| | | TAATACGACTCACTATAGGGAGAGTCTGCGCACATACGGGATAAAC |
| Pak | 10295 | TAATACGACTCACTATAGGGAGATCTTGGAGAAACTGCGCACCATTGT/ |
| | | TAATACGACTCACTATAGGGAGACTACCATCGTTGTGCGTTTGGATTG |
| Pak3 | 14895 | GCTTCTAATACGACTCACTATAGACCAGTACCGCCCAAGAAAT/ |
| | | GCTTCTAATACGACTCACTATAGGTTCCCTTGGGTCATCTGAAT |
| Par-1 | 30132 | TAATACGACTCACTATAGGGAGATGGCAGCAACTTTAAGCGACAGAACA/ |
| | | TAATACGACTCACTATAGGGAGAGTGGTGGAGCGACGTGGAATGAT |
| Par-1 | 30132 | TAATACGACTCACTATAGGGAGACAAGCAGAGCAAGCGCTACGGTGAA/ |
| | | TAATACGACTCACTATAGGGAGATCCTCACGCCGCTTAGACGCTGAAA |
| PDK | 8808 | TAATACGACTCACTATAGGGAGAATGTGGTTCGCGATGCTTACGAGAAT/ |
| | | TAATACGACTCACTATAGGGAGAATGATTGCATCTGTTCCGAATCCTT |
| PEK | 2087 | TAATACGACTCACTATAGGGAGACACCGCTTGTAGTCACGACTTTCAT/ |
| | | TAATACGACTCACTATAGGGAGAGCATCTGGATGTAGAGGTACACCTT |
| PhKgamma | 1830 | TAATACGACTCACTATAGGGAGATCTTCGACTATCTGACCTCTGTGGT/ |
| | | TAATACGACTCACTATAGGGAGACTTGACGGTTATACGTTGCGAAGGA |
| phl | 2845 | TAATACGACTCACTATAGGGAGAACTCTGCATGTGGAGGAGATCTTTG/ |
| | | TAATACGACTCACTATAGGGAGAGCATTATCAAACTGCGCTGCACTTC |
| Pitslre | 4268 | TAATACGACTCACTATAGGGAGAATGACGATGAGGAAAGCGAGGAGAG/ |
| | | TAATACGACTCACTATAGGGAGACGGGATAATAGTTGGGCAGGGGAAT |
| Pk17E | 7001 | TAATACGACTCACTATAGGGAGAACGTCTGGTCTGGTCACACTGCTAC/ |
| | | TAATACGACTCACTATAGGGAGACTGTTGTTGCTGCTGCTGCTGCTCCTGATA |
| Pk34A | 5182 | TAATACGACTCACTATAGGGAGAAGCCAGGGCGAAGCAGAAATTATGG/ |
| | | TAATACGACTCACTATAGGGAGATCGGTCATGTTTGTGGCTGGAGAAG |
| Pk61C | 1210 | TAATACGACTCACTATAGGGAGACGACCTCAAGCCCGAGAACATCCTG/ |
| | | TAATACGACTCACTATAGGGAGACACCAGGTCCTCGGCGTCCTTATCA |
| Pk61C | 1210 | TAATACGACTCACTATAGGGAGACGCGACCTCAAGCCCGAGAACATCC/ |
| | | TAATACGACTCACTATAGGGAGAGCACCAGGTCCTCGGCGTCCTTATC |
| Pk92B | 4720 | TAATACGACTCACTATAGGGAGAAGAAGGAGAACCACTTTCCGGACAT/ |
| | | TAATACGACTCACTATAGGGAGACTCCAGAAAGAAGTCCATCCAGAAC |
| Pka-C1 | 4379 | TAATACGACTCACTATAGGGAGATCGCTGCGCTACCACTTCAAGGACA/ |
| | | TAATACGACTCACTATAGGGAGACAGGTTGCGCAGTAGGTCCTTCAGA |
| Pka-C2 | 12066 | TAATACGACTCACTATAGGGAGACAACGGAAGTTTCGGCACTGTGATG/ |
| | | TAATACGACTCACTATAGGGAGACCACCAGTCCACCGATTTGTTGTAG |
| PKA-C3 | 6117 | TAATACGACTCACTATAGGGAGAGCCCGCTTCTGCACGCCTTTGTCATC/ |
| | | TAATACGACTCACTATAGGGAGAACTCGTCGTCGTCCTCCTCGTCATCGGTTTC |
| Pkc53E | 6622 | TAATACGACTCACTATAGGGATGGACCGTTTGTTCTTTGTAATGGA/ |
| | | TAATACGACTCACTATAGGGAGAGCTTATTTGGCTGCTTAGTTAGGAA |
| Pkc53E | 6622 | TAATACGACTCACTATAGGGAGACACCTTTCCTGGTCCAATTACACTC/ |
| | | TAATACGACTCACTATAGGGAGACTTTGCTCAGGCTCTTTGGATAGGA |
| Pkc98E | 1954 | TAATACGACTCACTATAGGGAGACGAAGCAGATGGCCGAGATACTCAG/ |
| | | TAATACGACTCACTATAGGGAGAAACGCAGTCAGGAAGGGATGGTTGG |
| PKCdelta | 10524 | TAATACGACTCACTATAGGGAGAAGCCCGAGAAGCCCGTGACT/ |
| | | TAATACGACTCACTATAGGGAGATGCGTTCGATGAGCGTGGAG |
| Pkg21D | 3324 | TAATACGACTCACTATAGGGAGACAGACGTTCTGGAGCTGGAGTTCTA/ |
| | | TAATACGACTCACTATAGGGAGAGGCAAAGATATCCACGCGATCCTGA |
| pll | 5974 | TAATACGACTCACTATAGGGAGAACGTTAGCGAGGATCTGCACAAGTA/ |
| | | TAATACGACTCACTATAGGGAGAGCTTACCACCTTTGATGCTGTATCC |
| png | 11420 | GCTTCTAATACGACTCACTATAGGGAACTGGGTGACTCTAAGCTG/ |
| | | GCTTCTAATACGACTCACTATAGACTCGAGTCCCACTACCATGTC |
| Polo | 12306 | TAATACGACTCACTATAGGGAGAGGAGTTCGAATGCCGCTACTACATT/ |
| | | TAATACGACTCACTATAGGGAGATCAGACAAGAGCTGGGCAAGAACAT |
| Polo | 12306 | TAATACGACTCACTATAGGGAGACGTTCTCCGCTTTGTGCTTGGTTTTCGTG/ |
| | | TAATACGACTCACTATAGGGAGACGCTTGTAGGTTTTCCGCTGGTTGATGTCG |
| PR2 | 3969 | TAATACGACTCACTATAGGGAGAACGAGAACATGCCGACAGTGGGTAA/ |
| | | TAATACGACTCACTATAGGGAGAGTTTCGGCAACGGACTTCCTGTTCA |
| put | 7904 | TAATACGACTCACTATAGGGAGAACGAGGCTGAGATAACAAACTCATC/ |
| | | TAATACGACTCACTATAGGGAGACTGGAATATCATGGCCAAACCAAAG |
| Pvr | 8222 | TAATACGACTCACTATAGGGAGATACAACGTTCAGGAATATGCCAATC/ |
| | | TAATACGACTCACTATAGGGAGAGTATATGCGTTCCACACTCAACTTT |
| Pvr | 8222 | TAATACGACTCACTATAGGGAGACCCTGCAAGAGCGCCATTATCCTG/ |
| | | TAATACGACTCACTATAGGGAGACTCTGTGTCCGGCATGGCTGGTTTA |
| Ret | 14396 | TAATACGACTCACTATAGGGAGATGACTACCGCTCACCAAACTCAAGT/ |
| | | TAATACGACTCACTATAGGGAGAGGGTCCATTATCATTGCGATCCAGT |
| Ret | 14396 | TAATACGACTCACTATAGGGAGAAGTTGCGAACTGAAGGTCAAGTCTC/ |
| | | TAATACGACTCACTATAGGGAGACATTCTGAAACCGGCCACATTTAGGA |
| rl | 12559 | TAATACGACTCACTATAGGGAGAGGCTGCCAAAAGACTGATGTA/ |
| | | TAATACGACTCACTATAGGGAGAGGAAGGAGAACCGCAAGATA |
| rok | 9774 | GCTTCTAATACGACTCACTATAGTGCGTCAACACAACTACAAGG/ |
| | | GCTTCTAATACGACTCACTATAGTTGTTCGCGACACATAGTACG |
| Ror | 4926 | TAATACGACTCACTATAGGGAGACTTTGCCCAGCTTGTGTTTCAGTTCA/ |
| | | TAATACGACTCACTATAGGGAGAGGCAATCCTCCACACCCACCATCC |
| S6k | 10539 | TAATACGACTCACTATAGGGAGAAAAGGTGGTTATGGCAAAGTATTTC/ |
| | | TAATACGACTCACTATAGGGAGAAAAATTTCAGGTGCCATGTACTCAA |
| S6kII | 17596 | TAATACGACTCACTATAGGGAGAATTTTGCCGCTGATTGGTGGAGTTT/ |
| | | TAATACGACTCACTATAGGGAGACAGCAGGAATAGGAGCTATACTATG |
| SAK | 7186 | TAATACGACTCACTATAGGGAGACGCTATATGAACCACATCGCCAGAC/ |
| | | TAATACGACTCACTATAGGGAGAAACATAAAGGGATGGCAGAGAACAG |
| SAK | 7186 | TAATACGACTCACTATAGGGAGAATACGGGAGGAATTTAAGCAAGTC/ |
| | | TAATACGACTCACTATAGGGAGATTATAACGCGTCGGAAGCAGTCT |
| SAX | 1891 | TAATACGACTCACTATAGGGAGACGCGATGCCGATGGTCAGGTGCAGGAG/ |
| | | TAATACGACTCACTATAGGGAGACCTCGTCCAATGCACTCGATCAGGG |
| sev | 18085 | TAATACGACTCACTATAGGGAGATGCAGAGTTTATTGGCGAACTGGAC/ |
| | | TAATACGACTCACTATAGGGAGAAAGCTTCCAGCATGCAGACGGATTA |
| sgg | 2621 | TAATACGACTCAGTATAGGGAGAATGCCAAGCCGAAGAACCGACTTTT/ |
| | | TAATACGACTCACTATAGGGAGACATCATCCACATCCTCTTGCACATC |
| shark | 18247 | TAATACGACTCACTATAGGGAGACAGTAGCTCAATGTTCAACACTCTG/ |
| | | TAATACGACTCACTATAGGGAGAATGAAGATAGCTGGCCATCTCACTT |
| Slob | 6772 | TAATACGACTCACTATAGGGAGAACCACCAGTGCCCGAAAAGAAAGTG/ |
| | | TAATACGACTCACTATAGGGAGAACCGAGGCATCTGTGACAAGAAACC |
| slpr | 2272 | GCTTCTAATACGACTCACTATAGCTCACCGTCCATTGCTTCTAC/ |
| | | GCTTCTAATACGACTCACTATAGGCACAACTGGGACTTTAGCAT |
| slpr | 2272 | TAATACGACTCACTATAGGGAGATTAAAAAGCGAAGGAAGCAAAGAGAAAACAACAAA/ |
| | | TAATACGACTCACTATAGGGAGATCCACCAGCCCACATCGCCAGACACC |
| smi35A | 4551 | TAATACGACTCACTATAGGGAGACCTGCAGCGTTGCTTGGAGTGGGAT/ |
| | | TAATACGACTCACTATAGGGAGATTTCCTGGTGGCCGCTGACGAGACA |
| SNF1A | 3051 | TAATACGACTCACTATAGGGAGATGTGAAGCACGGCAAGCTGCAGGAG/ |
| | | TAATACGACTCACTATAGGGAGAGTAGGCCGGGAGGTCCTTTTGGAAC |
| Src42A | 7873 | TAATACGACTCACTATAGGGAGAAGAACCGTGGTACTTCCGCAAAATC/ |
| | | TAATACGACTCACTATAGGGAGACATGATCTGGGCTTCCGCTAAGAAA |
| Src64B | 7524 | TAATACGACTCACTATAGGGAGAAGGAGTACATGTCCAAGGGCAGTCT/ |
| | | TAATACGACTCACTATAGGGAGAGCACTGGAGCAGCAGCTGATAAATG |
| SRPK | 8174 | TAATACGACTCACTATAGGGAGATCGAATTCAACGCTGCCAACACCTC/ |
| | | TAATACGACTCACTATAGGGAGAGGGCGTAAGGAACGAAGCGAATGAC |
| Strn-Mlck | 8304 | TAATACGACTCACTATAGGGAGATTCAGTGGTTTAAGGACAGCATTGA/ |
| | | TAATACGACTCACTATAGGGAGACAGGAAGCATGAAATCTTAACCTTG |
| Taf250 | 17603 | TAATACGACTCACTATAGGGAGAGCGGTTCGGGCCTGCACAGATTTGGTAT/ |
| | | TAATACGACTCACTATAGGGAGATTTGCTGGGCCTTTTTGCTTGATGCTC |
| Tak1 | 1388 | TAATACGACTCACTATAGGGAGACGACGTGGAGGCGAATGGCTTTGAT/ |
| | | TAATACGACTCACTATAGGGAGACTGCTTCTGTTCGCGCTCGGTTCGGTCCAT |
| Takl2 | 4803 | GCTTCTAATACGACTCACTATAGCAGCCGAAAGCAGTAATTCAT/ |
| | | GCTTCTAATACGACTCACTATAGTTGCCTTCATTAATAGCCATGT |
| Tie | 7525 | TAATACGACTCACTATAGGGAGATTGGTGGGGCAGAGAAAGAGGAG/ |
| | | TAATACGACTCACTATAGGGAGAGTCGCCGGCGGTCGCATTCAACTG |
| tkv | 14026 | TAATACGACTCACTATAGGGAGAGAACCATTGCCAAGCAGATTCAGAT/ |
| | | TAATACGACTCACTATAGGGAGATGAATGACATCCAGTTCCGAGTTGT |
| tor | 1389 | TAATACGACTCACTATAGGGAGACGGTTTGACGTTGGACAAGGTTCAT/ |
| | | TAATACGACTCACTATAGGGAGACATCTGGTTGCTAAAACGAGTGGAG |
| TOR | 5092 | TAATACGACTCACTATAGGGAGAGGCGCACTCGAATGCTTTGAAAAGG/ |
| | | TAATACGACTCACTATAGGGAGAGCTGACTTGGAAGCGACTGTTAGAG |
| trbl | 5408 | TAATACGACTCACTATAGGGAGACAAGCTCATCCAACAGCGTTATCTG/ |
| | | TAATACGACTCACTATAGGGAGAAAGTAGAACCGCTTGAGCTTGAGGT |
| trc | 8637 | TAATACGACTCACTATAGGGAGAAGAACTACTACAGCAACCTGGTGAC/ |
| | | TAATACGACTCACTATAGGGAGAGCCGTCTCACTGATATAGAACTGTG |
| trc | 8637 | TAATACGACTCACTATAGGGAGAATGAGCAGCAGAACGCAGGAC/ |
| | | TAATACGACTCACTATAGGGAGATCGCTTCAGCCGGAGATACT |
| twf | 3172 | TAATACGACTCACTATAGGGAGATCGGATCAGCATACATCACAGAGGA/ |
| | | TAATACGACTCACTATAGGGAGAGAGAAAAGGAGCCTTACAGCTTGAG |
| wee | 4488 | TAATACGACTCACTATAGGGAGAGATAGAGGGCCTACGCTATATTCAT/ |
| | | TAATACGACTCACTATAGGGAGAATATAGACTGCGAAGTGGGCCTCTT |
| wee | 4488 | TAATACGACTCACTATAGGGAGAGCATCGGGTACGGCCACATTATTA/ |
| | | TAATACGACTCACTATAGGGAGACGCCGCCTTCTTTGCCTATCTTAC |
| wit | 10776 | TAATACGACTCACTATAGGGAGACAGATACCTCTAGCTGCCTTGGAAC/ |
| | | TAATACGACTCACTATAGGGAGACGGAGGTTTATCGAGGCGAGGATTA |
| wts | 12072 | TAATACGACTCACTATAGGGAGAAACAGCAACTGCAGGCCTTGAGGGT/ |
| | | TAATACGACTCACTATAGGGAGAATACGTGCGCTGGCGATACGACTTG |

TABLE 4

List of Drosophila protein kinase regulators studied
in this work and primers used to synthesize
dsRNA. All primers led to the synthesis of a single
band of dsRNA. Name and CG number are indicated.

| | | |
|---|---|---|
| | | FORWARD SEQUENCE/ |
| NAME | CG | REVERSE SEQUENCE |

| | | |
|---|---|---|
| CkIIbeta | 15224 | TAATACGACTCACTATAGGGAGATGGGTCACCTGGTTCTGTGGACTTC/ |
| | | TAATACGACTCACTATAGGGAGAGACGCTTGGGACGATATTCGGGATG |
| SNF4Agamma | 17299 | TAATACGACTCACTATAGGGAGACGCCGCCGAGAAAACCTACAAC/ |
| | | TAATACGACTCACTATAGGGAGACCGGCGCCGTCTCCTCTTC |
| PVF1 | 7103 | TAATACGACTCACTATAGGGAGATGTCCTCTAACGCCATTGAAAACT/ |
| | | TAATACGACTCACTATAGGGAGAGTGGCGGCGGCGTAGAAGAACC |
| PVF2 | 13780 | TAATACGACTCACTATAGGGAGATATCGCGATCGGAGTGCTAAT/ |
| | | TAATACGACTCACTATAGGGAGAGACCGCTCGATCCTCAAAGTA |
| PVF3 | 13782 | TAATACGACTCACTATAGGGAGATGAGACTGCGGCTTGCCTTGATTTTCCTA/ |
| | | TAATACGACTCACTATAGGGAGATGAGACGCCGGTTTCGATGGTGTGC |
| CyclinE | 3938 | TAATACGACTCACTATAGGGAGACGTGCCATTCTCTTGGACTGGTTGA/ |
| | | TAATACGACTCACTATAGGGAGACTGCCAGCACCGAGTAGGAATAGTT |
| CyclinD | 9096 | TAATACGACTCACTATAGGGAGATGAGAGTGCGGCGATCCATAGAAT/ |
| | | TAATACGACTCACTATAGGGAGACTCCAGACACCGATCCGAATACAA |

TABLE 5
Codes used for quantitation of mitotic phenotypes.

| Description of phenotype | Defect code |
|---|---|
| Centrosomal defects | |
| Centrosome number zero | CN0 |
| Single centrosome | CN1 |
| Centrosome number high | CNH |
| (3-5) | |
| Centrosome number very high | CNVH |
| (>5) | |
| Centrosome Position Defects | CPD |
| Spindle Defects | |
| Monopolar | SMO |
| Tripolar | STR |
| Multipolar | SMP |
| Multipolar Cytokinesis | MC |
| Abnormal | AS |
| Branched | SBR |
| Splayed pole | SSP |
| No astral microtubules | NAS |
| Central Spindle defects | CSD |
| Chromosome defects | |
| Chromosome condensation | CRCD |
| defect | |
| Chromosome number high | CRNH |
| Lagging chromatids | CRLC |
| Chromosome alignment defect | CRAD |
| Chromosome segregation | CRSD |
| defects | |
| Uneven DNA | UD |

1. a method of modulating proliferation in a cell or population of cells, comprising contacting said cell or population of cells with an agent capable of modulating expression or activity of a target kinase or regulator of Table 1.

2. A method of screening for a modulator of cell proliferation, comprising determining the effect of a candidate substance on the expression or activity of a target kinase or regulator of Table 1, said method optionally comprising determining the effect of the candidate substance on proliferation (e. g. division) of a cell or population of cells.

3. A method according to claim 2 comprising contacting a cell capable of expressing the target kinase with the candidate substance, said method optionally comprising determining the effect of the candidate substance on proliferation (e. g. division) of a cell or population of cells.

4. A method according to claim 3 wherein the cell is capable of expressing the target kinase or regulator from an endogenous coding sequence, said method optionally comprising determining the effect of the candidate substance on proliferation (e. g. division) of a cell or population of cells.

5. A method according to claim 3 wherein the cell is capable of expressing the target kinase or regulator from an exogenous coding sequence, said method optionally comprising determining the effect of the candidate substance on proliferation (e. g. division) of a cell or population of cells.

6. A method according to claim 2 comprising contacting the target kinase protein with the candidate substance in a cell-free system, said method optionally comprising determining the effect of the candidate substance on proliferation (e. g. division) of a cell or population of cells.

7. (canceled)

8. A method according to claim 2, further comprising determining the extent to which apoptosis occurs in the cell or population of cells.

9. A method according to claim 2 wherein the modulator is an inhibitor of expression or activity of the target kinase or regulator.

10. A method according to claim 9 wherein the modulator is a nucleic acid molecule.

11. A method according to claim 10 wherein the nucleic acid molecule is, or encodes, anti-sense RNA or DNA, a triple helix-forming molecule, RNAi, siRNA or a ribozyme.

12. A method of determining the effect of a candidate substance on proliferation of a cell or population of cells, comprising contacting said cell or population of cells with said candidate substance, said candidate substance having previously been identified as a modulator of activity or expression of a target kinase of Table 1.

13. A method of preparing a pharmaceutical composition for the treatment of a proliferative disorder, the method comprising, having identified a modulator of proliferation, or a modulator of target kinase or regulator expression or activity, by a method according to claim 2, formulating said modulator with a pharmaceutically acceptable carrier.

14. A method of treatment of a proliferative disorder in a subject suffering therefrom, comprising administering to said subject a modulator of expression or activity of a target kinase or regulator of Table 1.

15-17. (canceled)

18. A method according to claim 13 wherein the proliferative disorder is cancer, psoriasis or glomerulonephritis.

19. A method of diagnosis of a proliferative disorder, comprising contacting a cell or population of cells, or an extract thereof, with a binding agent capable of binding specifically to a target kinase or regulator of Table 1.

20. A method according to claim 19 wherein the binding agent binds to the target kinase or regulator protein.

21. A method according to claim 19 wherein the binding agent binds to RNA encoding the target kinase or regulator.

22. A method according to claim 19 wherein the proliferative disorder is selected from the group consisting of cancer, psoriasis or glomerulonephritis.

23. A method for identifying a kinase which is abnormally expressed in a proliferative disorder, comprising contacting a cell or population of cells affected by the disorder with a plurality of binding agents each capable of binding specifically and independently to a kinase, wherein at least one of said kinases is a target kinase of Table 1.

24. A method according to claim 23 wherein the cell or cells are contacted with binding agents capable of binding specifically and independently to a plurality of kinases of Table 1.

25. A method according to claim 24 wherein the cell or cells are contacted with binding agents capable of binding specifically and independently to at least 2, 5, 10, 15, 20, 25, 30, 40, 50, 60, 70 or to substantially all of the target kinases of Table 1.

26. A vector comprising a coding sequence for a kinase or regulator of Table 1 operably linked to transcriptional regulatory sequences for use in a method of gene therapy.

27. A vector according to claim 26 for use in the treatment of proliferative disease.

28. A method of treatment of a proliferative disorder in a subject suffering therefrom, comprising administering to said subject a vector according to claim 26.

29. A medicament comprising a vector according to claim 26 in a pharmaceutically acceptable carrier for the treatment of a proliferative disorder.

30. The medicament of claim 29 wherein the proliferative disorder is cancer, psoriasis or glomerulonephritis.

31. (canceled)

32. A method according to claim 14, wherein the proliferative disorder is cancer, psoriasis or glomerulonephritis


SEQUENCE LISTING (556 sequences)

SEQ ID NO: 1 - DNA, 41 bp, Artificial sequence
  T7 oligonucleotide
taatacgactcactatagggggcagaaaggcggcaaattgt

SEQ ID NO: 2 - DNA, 42 bp, Artificial sequence
  T7 oligonucleotide
taatacgactcactatagggccaacgagctgataagcgataa

SEQ ID NO: 3 - DNA, 43 bp, Artificial sequence
  T7 oligonucleotide
taatacgactcactatagggcattcacactggtttggaagttg

SEQ ID NO: 4 - DNA, 40 bp, Artificial sequence
  T7 oligonucleotide
taatacgactcactatagggcccagggaccaaacatcaga

SEQ ID NO: 5 - DNA, 42 bp, Artificial sequence
  T7 oligonucleotide
taatacgactcactatagggaggatcttattcgaaggctcat

SEQ ID NO: 6 - DNA, 43 bp, Artificial sequence
  T7 oligonucleotide
taatacgactcactataggggttagtggaccatcaacagttga

SEQ ID NO: 7 - DNA, 40 bp, Artificial sequence
  T7 oligonucleotide
taatacgactcactataggggcgctgcactacgcctttca

SEQ ID NO: 8 - DNA, 41 bp, Artificial sequence
  T7 oligonucleotide
taatacgactcactatagggatgggaactggaatcgctctt

SEQ ID NO: 9 - DNA, 40 bp, Artificial sequence
  T7 oligonucleotide
taatacgactcactatagggcctctgggcaaaggcaagtt

SEQ ID NO: 10 - DNA, 41 bp, Artificial sequence
  T7 oligonucleotide
taatacgactcactatagggatgcgcccctcaatcatctct

SEQ ID NO: 11 - DNA, 41 bp, Artificial sequence
  T7 oligonucleotide
taatacgactcactatagggattgtgcttggctgccagtac

SEQ ID NO: 12 - DNA, 41 bp, Artificial sequence
  T7 oligonucleotide
taatacgactcactatagggtcgaaaaccttggtggaatgg

SEQ ID NO: 13 - DNA, 24 bp, Artificial sequence
  Oligo
catattaaactgacggatttggcc

SEQ ID NO: 14 - DNA, 25 bp, Artificial sequence
  Oligo
ggccaaaatccgtcagtttaatatg

SEQ ID NO: 15 - DNA, 24 bp, Artificial sequence
  Oligo
aggatcatttgctggtgtctacag

SEQ ID NO: 16 - DNA, 30 bp, Artificial sequence
  Oligo
gaaggatgtttcaattggcaatgtattttc

SEQ ID NO: 17 - DNA, 24 bp, Artificial sequence
  Oligo
gtacctccttgatggtgggtttaa

SEQ ID NO: 18 - DNA, 21 bp, Artificial sequence
  Oligo
tggacaagtggttggagcttt

SEQ ID NO: 19 - DNA, 23 bp, Artificial sequence
  Oligo
acctatgggaagatcatgaacca

SEQ ID NO: 20 - DNA, 22 bp, Artificial sequence
  Oligo
atgaggtccttcgcttcttcag

SEQ ID NO: 21 - DNA, 22 bp, Artificial sequence
  Oligo
gcagaagagctgcacatttgac

SEQ ID NO: 22 - DNA, 24 bp, Artificial sequence
  Oligo
ccatggcagtacattagagcatct

SEQ ID NO: 23 - DNA, 18 bp, Artificial sequence
  Oligo
aacggcagcgtgcagatc

SEQ ID NO: 24 - DNA, 18 bp, Artificial sequence
  Oligo
ggtcacggctgccatcag

SEQ ID NO: 25 - DNA, 48 bp, Artificial sequence
  Primer
taatacgactcactatagggagacacgggcgatagtctggagcagagt

SEQ ID NO: 26 - DNA, 48 bp, Artificial sequence
  Primer
taatacgactcactatagggagacggaatggggctggccttcggattt

SEQ ID NO: 27 - DNA, 48 bp, Artificial sequence
  Primer
taatacgactcactatagggagatctactcgaatttcaaccagtctct

SEQ ID NO: 28 - DNA, 48 bp, Artificial sequence
  Primer
taatacgactcactatagggagatcataccaaatacgattcaccacac

SEQ ID NO: 29 - DNA, 51 bp, Artificial sequence
  Primer
taatacgactcactatagggagattacatcgggtcatgcgcttacggaaca

SEQ ID NO: 30 - DNA, 48 bp, Artificial sequence
  Primer
taatacgactcactatagggagacactttcttaacgccgctgctatta

SEQ ID NO: 31 - DNA, 48 bp, Artificial sequence
  Primer
taatacgactcactatagggagacatcgagacggagatgctgtggaaa

SEQ ID NO: 32 - DNA, 48 bp, Artificial sequence
  Primer
taatacgactcactatagggagacgaggtgaatatgccatcgaggaag

SEQ ID NO: 33 - DNA, 44 bp, Artificial sequence
  Primer
gcttctaatacgactcactataggctctccttccacaacgaaat

SEQ ID NO: 34 - DNA, 45 bp, Artificial sequence
  Primer
gcttctaatacgactcactatagaaccacaaaaagtatgcacaaa

SEQ ID NO: 35 - DNA, 45 bp, Artificial sequence
  Primer
taatacgactcactatagggagagcagcgcaagcaacaacaacta

SEQ ID NO: 36 - DNA, 53 bp, Artificial sequence
  Primer
taatacgactcactatagggagagatggtaaatggctaaacaaaacgctcaat

SEQ ID NO: 37 - DNA, 48 bp, Artificial sequence
  Primer
taatacgactcactatagggagaacgtgcgcatatatctgatcttgga

SEQ ID NO: 38 - DNA, 48 bp, Artificial sequence
  Primer
taatacgactcactatagggagaattaaggaccagcagcttggaaatg

SEQ ID NO: 39 - DNA, 48 bp, Artificial sequence; identical to SEQ ID NO: 37
  Primer

SEQ ID NO: 40 - DNA, 48 bp, Artificial sequence; identical to SEQ ID NO: 38
  Primer

SEQ ID NO: 41 - DNA, 44 bp, Artificial sequence
  Primer
gcttctaatacgactcactatagacacacaattggtcgctcaaa

SEQ ID NO: 42 - DNA, 44 bp, Artificial sequence
  Primer
gcttctaatacgactcactataggattaggagcatgtgcctgtg

SEQ ID NO: 43 - DNA, 49 bp, Artificial sequence
  Primer
taatacgactcactatagggagaacaatggaacttggacacagttgtgg

SEQ ID NO: 44 - DNA, 48 bp, Artificial sequence
  Primer
taatacgactcactatagggagacatcgtaatacggcaattgatactc

SEQ ID NO: 45 - DNA, 48 bp, Artificial sequence
  Primer
taatacgactcactatagggagactcgcagcagctggttgtccacacc

SEQ ID NO: 46 - DNA, 50 bp, Artificial sequence
  Primer
taatacgactcactatagggagaccgctctcgtcgcctgtctgattcaaa

SEQ ID NO: 47 - DNA, 48 bp, Artificial sequence
  Primer
taatacgactcactatagggagactgccagtcagcgacaacaagaaga

SEQ ID NO: 48 - DNA, 48 bp, Artificial sequence
  Primer
taatacgactcactatagggagattgttgctgctgttgctgctgggat

SEQ ID NO: 49 - DNA, 48 bp, Artificial sequence
  Primer
taatacgactcactatagggagaccaacatactgctgggcctggaaaa

SEQ ID NO: 50 - DNA, 48 bp, Artificial sequence
  Primer
taatacgactcactatagggagagctgctggtctgtggcttcatctta

SEQ ID NO: 51 - DNA, 48 bp, Artificial sequence
  Primer
taatacgactcactatagggagataaggccaagactttctgctatctt

SEQ ID NO: 52 - DNA, 48 bp, Artificial sequence
  Primer
taatacgactcactatagggagaaaatcagccatgcaccttagtgttt

SEQ ID NO: 53 - DNA, 48 bp, Artificial sequence
  Primer
taatacgactcactatagggagatcgacttcggcctggcgtctaagtt

SEQ ID NO: 54 - DNA, 48 bp, Artificial sequence
  Primer
taatacgactcactatagggagatgtcacacttgctgccatccaccac

SEQ ID NO: 55 - DNA, 48 bp, Artificial sequence
  Primer
taatacgactcactatagggagacagctggaccaccaaaacattgtca

SEQ ID NO: 56 - DNA, 48 bp, Artificial sequence
  Primer
taatacgactcactatagggagattgatggcagttgactcgctgttac

SEQ ID NO: 57 - DNA, 55 bp, Artificial sequence
  Primer
taatacgactcactatagggagaaccacccacacctcatctcgcccacccacacg

SEQ ID NO: 58 - DNA, 52 bp, Artificial sequence
  Primer
taatacgactcactatagggagacggcggcgacagcgacagcgagaggaagt

SEQ ID NO: 59 - DNA, 46 bp, Artificial sequence
  Primer
taatacgactcactatagggagaattggcgagtgaccctgctggac

SEQ ID NO: 60 - DNA, 47 bp, Artificial sequence
  Primer
taatacgactcactatagggagagggggtaacgggctcaaagggtag

SEQ ID NO: 61 - DNA, 48 bp, Artificial sequence
  Primer
taatacgactcactatagggagatttacactcagcaggaattattcac

SEQ ID NO: 62 - DNA, 48 bp, Artificial sequence
  Primer
taatacgactcactatagggagatctggatcaatgactagcattttac

SEQ ID NO: 63 - DNA, 48 bp, Artificial sequence
  Primer
taatacgactcactatagggagacggaccacttcaaatatcagatgtg

SEQ ID NO: 64 - DNA, 48 bp, Artificial sequence
  Primer
taatacgactcactatagggagaggaaactcggaatttgtaggtttgg

SEQ ID NO: 65 - DNA, 48 bp, Artificial sequence
  Primer
taatacgactcactatagggagattggatcgggacagtttggtgttgt

SEQ ID NO: 66 - DNA, 48 bp, Artificial sequence
  Primer
taatacgactcactatagggagatcttagaggagaagcgcgtgtagtt

SEQ ID NO: 67 - DNA, 46 bp, Artificial sequence
  Primer
taatacgactcactatagggagagtgatattatttcgaccgcaact

SEQ ID NO: 68 - DNA, 53 bp, Artificial sequence
  Primer
taatacgactcactatagggagaagcaacttcagggatcgactctcattcagg

SEQ ID NO: 69 - DNA, 48 bp, Artificial sequence
  Primer
taatacgactcactatagggagaaaaatccatcatgccacgcagagtt

SEQ ID NO: 70 - DNA, 48 bp, Artificial sequence
  Primer
taatacgactcactatagggagagctcagaggtttccaatatggtaga

SEQ ID NO: 71 - DNA, 44 bp, Artificial sequence
  Primer
taatacgactcactatagggagaccagtaggcgcaatgtaggtc

SEQ ID NO: 72 - DNA, 45 bp, Artificial sequence
  Primer
taatacgactcactatagggagattgcgatggataaatggatgct

SEQ ID NO: 73 - DNA, 47 bp, Artificial sequence
  Primer
taatacgactcactatagggagaccagctgcccggtgttcaatccat

SEQ ID NO: 74 - DNA, 46 bp, Artificial sequence
  Primer
taatacgactcactatagggagacagaggcgtcggcggcacaagta

SEQ ID NO: 75 - DNA, 48 bp, Artificial sequence
  Primer
taatacgactcactatagggagatctgttaccttcaagatagttcctt

SEQ ID NO: 76 - DNA, 48 bp, Artificial sequence
  Primer
taatacgactcactatagggagatcgatatcaaggtgttcttaatgtg

SEQ ID NO: 77 - DNA, 48 bp, Artificial sequence
  Primer
taatacgactcactatagggagacgagggaatgctctacatggtcttc

SEQ ID NO: 78 - DNA, 48 bp, Artificial sequence
  Primer
taatacgactcactatagggagacgttcgccgagattgatttccactc

SEQ ID NO: 79 - DNA, 48 bp, Artificial sequence
  Primer
taatacgactcactatagggagatggttactggtggagaactctttga

SEQ ID NO: 80 - DNA, 48 bp, Artificial sequence
  Primer
taatacgactcactatagggagatgcttgcttgcaagtatacctcttc

SEQ ID NO: 81 - DNA, 48 bp, Artificial sequence
  Primer
taatacgactcactatagggagaattacattggcgagctttactttac

SEQ ID NO: 82 - DNA, 46 bp, Artificial sequence
  Primer
taatacgactcactatagggagaaacttactttcccaactcttctt

SEQ ID NO: 83 - DNA, 47 bp, Artificial sequence
  Primer
taatacgactcactatagggagaaaatttcgttgcttaaggagttga

SEQ ID NO: 84 - DNA, 43 bp, Artificial sequence
  Primer
taatacgactcactatagggagaatcgacgggacaggaatacc

SEQ ID NO: 85 - DNA, 47 bp, Artificial sequence
  Primer
taatacgactcactatagggagaaaggattgtttacgaggttatgac

SEQ ID NO: 86 - DNA, 44 bp, Artificial sequence
  Primer
taatacgactcactatagggagattgttgccggaaatgactacg

SEQ ID NO: 87 - DNA, 50 bp, Artificial sequence
  Primer
taatacgactcactatagggagaaggatgtccagcttaaagtcaaacgat

SEQ ID NO: 88 - DNA, 53 bp, Artificial sequence
  Primer
taatacgactcactatagggagacccaccaccacctcacgcaagcggacaata

SEQ ID NO: 89 - DNA, 53 bp, Artificial sequence
  Primer
taatacgactcactatagggagatctctattacgaagcaagcaaaaagtgatt

SEQ ID NO: 90 - DNA, 53 bp, Artificial sequence
  Primer
taatacgactcactatagggagaccagatgaaaggggaggcggccgatgagtc

SEQ ID NO: 91 - DNA, 49 bp, Artificial sequence
  Primer
taatacgactcactatagggagacgaacaaggcaaataaatcaagtcac

SEQ ID NO: 92 - DNA, 43 bp, Artificial sequence
  Primer
taatacgactcactatagggagagggggcaaatcgtcggaata

SEQ ID NO: 93 - DNA, 48 bp, Artificial sequence
  Primer
taatacgactcactatagggagacactgcgaccaggacctcaagaagt

SEQ ID NO: 94 - DNA, 48 bp, Artificial sequence
  Primer
taatacgactcactatagggagaccacgaggtgatggccggaaaagat

SEQ ID NO: 95 - DNA, 48 bp, Artificial sequence
  Primer
taatacgactcactatagggagatggtggacgttttcggtcaactttc

SEQ ID NO: 96 - DNA, 48 bp, Artificial sequence
  Primer
taatacgactcactatagggagacagtttgctcaaatgcggccattca

SEQ ID NO: 97 - DNA, 48 bp, Artificial sequence
  Primer
taatacgactcactatagggagactttccgcaggacaaggactgggag

SEQ ID NO: 98 - DNA, 48 bp, Artificial sequence
  Primer
taatacgactcactatagggagatgctgttgctgctgctgctggtggt

SEQ ID NO: 99 - DNA, 48 bp, Artificial sequence
  Primer
taatacgactcactatagggagaagctgccaacgtgctgattaccaag

SEQ ID NO: 100 - DNA, 48 bp, Artificial sequence
  Primer
taatacgactcactatagggagatgggcatgggatccgtccagaagaa

SEQ ID NO: 101 - DNA, 48 bp, Artificial sequence
  Primer
taatacgactcactatagggagaatggtggactcttgtaaacgttggg

SEQ ID NO: 102 - DNA, 48 bp, Artificial sequence
  Primer
taatacgactcactatagggagaggatgaagagcaaaccagcagattc

SEQ ID NO: 103 - DNA, 48 bp, Artificial sequence
  Primer
taatacgactcactatagggagaaggagacggaattgcgccagaaact

SEQ ID NO: 104 - DNA, 48 bp, Artificial sequence
  Primer
taatacgactcactatagggagaagctctctcttgggtggcaaacagt

SEQ ID NO: 105 - DNA, 48 bp, Artificial sequence
  Primer
taatacgactcactatagggagacgccgttctggaaatgcaaggagtg

SEQ ID NO: 106 - DNA, 48 bp, Artificial sequence
  Primer
taatacgactcactatagggagagacgtttccagcacaggcttgtgac

SEQ ID NO: 107 - DNA, 48 bp, Artificial sequence
  Primer
taatacgactcactatagggagaaaaggagaagcctgcctgatcaagg

SEQ ID NO: 108 - DNA, 48 bp, Artificial sequence
  Primer
taatacgactcactatagggagacgtgctcgaagatatacggctgttc

SEQ ID NO: 109 - DNA, 48 bp, Artificial sequence
  Primer
taatacgactcactatagggagagatttcaaaaccatccgcaccaaac

SEQ ID NO: 110 - DNA, 48 bp, Artificial sequence
  Primer
taatacgactcactatagggagaggtgacggatcttaaactctgatac

SEQ ID NO: 111 - DNA, 48 bp, Artificial sequence
  Primer
taatacgactcactatagggagaatacgcagcggaagcaatcgacttg

SEQ ID NO: 112 - DNA, 48 bp, Artificial sequence
  Primer
taatacgactcactatagggagaagttgatggcactggagatctgttc

SEQ ID NO: 113 - DNA, 48 bp, Artificial sequence
  Primer
taatacgactcactatagggagaagcgcaagagcagtgtgagcagtga

SEQ ID NO: 114 - DNA, 48 bp, Artificial sequence
  Primer
taatacgactcactatagggagacgccagcacaaagttcagcttggac

SEQ ID NO: 115 - DNA, 48 bp, Artificial sequence
  Primer
taatacgactcactatagggagaacgtggagctgccgctgatgacctt

SEQ ID NO: 116 - DNA, 48 bp, Artificial sequence
  Primer
taatacgactcactatagggagagcttcaccttgtgcaccagcagacc

SEQ ID NO: 117 - DNA, 48 bp, Artificial sequence
  Primer
taatacgactcactatagggagatggctacgactgtgtggcagacata

SEQ ID NO: 118 - DNA, 48 bp, Artificial sequence
  Primer
taatacgactcactatagggagacaccttgctctgcatgcgctatcat

SEQ ID NO: 119 - DNA, 45 bp, Artificial sequence
  Primer
taatacgactcactatagggagaggcaacccaggaaaacggaatg

SEQ ID NO: 120 - DNA, 42 bp, Artificial sequence
  Primer
taatacgactcactatagggagaggagcagctgccttggaca

SEQ ID NO: 121 - DNA, 47 bp, Artificial sequence
  Primer
taatacgactcactatagggagaaaatcaatcatgccagccttcttc

SEQ ID NO: 122 - DNA, 50 bp, Artificial sequence
  Primer
taatacgactcactatagggagacatcttgacggactccttagccttgac

SEQ ID NO: 123 - DNA, 48 bp, Artificial sequence
  Primer
taatacgactcactatagggagatttctacgggcaaagaggctaatgt

SEQ ID NO: 124 - DNA, 48 bp, Artificial sequence
  Primer
taatacgactcactatagggagaaggaagtcaaaggaatgcggatgat

SEQ ID NO: 125 - DNA, 48 bp, Artificial sequence
  Primer
taatacgactcactatagggagatggatcaggcagatgaggaggatga

SEQ ID NO: 126 - DNA, 48 bp, Artificial sequence
  Primer
taatacgactcactatagggagaagacggagctattgtgctggttgtg

SEQ ID NO: 127 - DNA, 48 bp, Artificial sequence
  Primer
taatacgactcactatagggagaggaaattgtggaaggcacaccatac

SEQ ID NO: 128 - DNA, 48 bp, Artificial sequence
  Primer
taatacgactcactatagggagaaagcaggagaatggacatcgactca

SEQ ID NO: 129 - DNA, 48 bp, Artificial sequence
  Primer
taatacgactcactatagggagacacggtcaacctgatagcctcctac

SEQ ID NO: 130 - DNA, 48 bp, Artificial sequence
  Primer
taatacgactcactatagggagacaccaaatgacgcagagctccactg

SEQ ID NO: 131 - DNA, 48 bp, Artificial sequence
  Primer
taatacgactcactatagggagatcatgagaaggaccagcgacaagat

SEQ ID NO: 132 - DNA, 48 bp, Artificial sequence
  Primer
taatacgactcactatagggagaccgaagtcaatcatgtacacctgag

SEQ ID NO: 133 - DNA, 48 bp, Artificial sequence
  Primer
taatacgactcactatagggagacagtctaatcgaccttggggaaaac

SEQ ID NO: 134 - DNA, 48 bp, Artificial sequence
  Primer
taatacgactcactatagggagaataactctggagcccggtagacaat

SEQ ID NO: 135 - DNA, 48 bp, Artificial sequence
  Primer
taatacgactcactatagggagaaggagcatagcggaccgtacacaaa

SEQ ID NO: 136 - DNA, 48 bp, Artificial sequence
  Primer
taatacgactcactatagggagagagttatccaaagctgtggcactgg

SEQ ID NO: 137 - DNA, 47 bp, Artificial sequence
  Primer
taatacgactcactatagggagaagtggcagcgctgtgggaataacg

SEQ ID NO: 138 - DNA, 47 bp, Artificial sequence
  Primer
taatacgactcactatagggagacacccgatgctgcactgatggaca

SEQ ID NO: 139 - DNA, 48 bp, Artificial sequence
  Primer
taatacgactcactatagggagagctgcggcacagaatcatcaccata

SEQ ID NO: 140 - DNA, 48 bp, Artificial sequence
  Primer
taatacgactcactatagggagatgactgcattggagatggcctgttg

SEQ ID NO: 141 - DNA, 48 bp, Artificial sequence
  Primer
taatacgactcactatagggagatcacaagatcggcgaggggtcttat

SEQ ID NO: 142 - DNA, 48 bp, Artificial sequence
  Primer
taatacgactcactatagggagaaagcagctgatccgcagtatgtctc

SEQ ID NO: 143 - DNA, 48 bp, Artificial sequence
  Primer
taatacgactcactatagggagagcagtcagagatgcaggagcaggaa

SEQ ID NO: 144 - DNA, 48 bp, Artificial sequence
  Primer
taatacgactcactatagggagaatcgcgccaggcattccagctcaaa

SEQ ID NO: 145 - DNA, 48 bp, Artificial sequence
  Primer
taatacgactcactatagggagaggctcaaagcgaatgtggctaccaa

SEQ ID NO: 146 - DNA, 49 bp, Artificial sequence
  Primer
taatacgactcactatagggagacctggtgatgatgatgtccatgactg

SEQ ID NO: 147 - DNA, 48 bp, Artificial sequence
  Primer
taatacgactcactatagggagacagacgacgaaccagcagcaacaac

SEQ ID NO: 148 - DNA, 48 bp, Artificial sequence
  Primer
taatacgactcactatagggagagggaagcatggttcatggcagtggt

SEQ ID NO: 149 - DNA, 48 bp, Artificial sequence
  Primer
taatacgactcactatagggagatgcgacatcccctcagagcgttcct

SEQ ID NO: 150 - DNA, 50 bp, Artificial sequence
  Primer
taatacgactcactatagggagaatttgattcttagtgcctttcttgtcg

SEQ ID NO: 151 - DNA, 48 bp, Artificial sequence
  Primer
taatacgactcactatagggagacgtctgtcaaggctctggtgaagaa

SEQ ID NO: 152 - DNA, 48 bp, Artificial sequence
  Primer
taatacgactcactatagggagatgatccaggctcaagttgttggtgg

SEQ ID NO: 153 - DNA, 48 bp, Artificial sequence
  Primer
taatacgactcactatagggagacaacatcacctctagatcgagttta

SEQ ID NO: 154 - DNA, 48 bp, Artificial sequence
  Primer
taatacgactcactatagggagatgaatcagctagaaacggcacattt

SEQ ID NO: 155 - DNA, 48 bp, Artificial sequence
  Primer
taatacgactcactatagggagaccagagccaaagcacctacgatgac

SEQ ID NO: 156 - DNA, 48 bp, Artificial sequence
  Primer
taatacgactcactatagggagaatgcagcaggcaatccttggtggtg

SEQ ID NO: 157 - DNA, 48 bp, Artificial sequence
  Primer
taatacgactcactatagggagatgtacgaggtgattgctcagaatcc

SEQ ID NO: 158 - DNA, 48 bp, Artificial sequence
  Primer
taatacgactcactatagggagaatgggcttgtcctggaagtaccact

SEQ ID NO: 159 - DNA, 48 bp, Artificial sequence
  Primer
taatacgactcactatagggagacggggttaagacacttagctatttg

SEQ ID NO: 160 - DNA, 48 bp, Artificial sequence
  Primer
taatacgactcactatagggagactctgcagacacagtttcttgattc

SEQ ID NO: 161 - DNA, 48 bp, Artificial sequence
  Primer
taatacgactcactatagggagagctggatctgttcatcgcgcacttg

SEQ ID NO: 162 - DNA, 48 bp, Artificial sequence
  Primer
taatacgactcactatagggagacaaactgggatcctcggagaccttc

SEQ ID NO: 163 - DNA, 50 bp, Artificial sequence
  Primer
taatacgactcactatagggagagttataccacagttggggaagctttac

SEQ ID NO: 164 - DNA, 49 bp, Artificial sequence
  Primer
taatacgactcactatagggagattcttcagtgccttaatagcgtagta

SEQ ID NO: 165 - DNA, 48 bp, Artificial sequence
  Primer
taatacgactcactatagggagaaagagctggaccaaactgtggaaag

SEQ ID NO: 166 - DNA, 48 bp, Artificial sequence
  Primer
taatacgactcactatagggagatcatcatagatgcgtctcgaggaga

SEQ ID NO: 167 - DNA, 48 bp, Artificial sequence
  Primer
taatacgactcactatagggagagcgcaagatcggctgtggatccttc

SEQ ID NO: 168 - DNA, 48 bp, Artificial sequence
  Primer
taatacgactcactatagggagaccaatggaggcgtacctggctgttc

SEQ ID NO: 169 - DNA, 48 bp, Artificial sequence
  Primer
taatacgactcactatagggagagataaagttcttgctacagtggaaa

SEQ ID NO: 170 - DNA, 48 bp, Artificial sequence
  Primer
taatacgactcactatagggagaaaaggtaaagcatttgaatcaggag

SEQ ID NO: 171 - DNA, 48 bp, Artificial sequence
  Primer
taatacgactcactatagggagatgtgacgctttacgttctgatgttt

SEQ ID NO: 172 - DNA, 48 bp, Artificial sequence
  Primer
taatacgactcactatagggagatcgcgtaaagagtgtccattttgtt

SEQ ID NO: 173 - DNA, 48 bp, Artificial sequence
  Primer
taatacgactcactatagggagatctaccaaatcctgccgcgtcctgt

SEQ ID NO: 174 - DNA, 48 bp, Artificial sequence
  Primer
taatacgactcactatagggagaggtggccgaggacacatgtatcttg

SEQ ID NO: 175 - DNA, 51 bp, Artificial sequence
  Primer
taatacgactcactatagggagagattgtgctgattctcctgctggtgcta

SEQ ID NO: 176 - DNA, 50 bp, Artificial sequence
  Primer
taatacgactcactatagggagaaagaacattgtacgaagtgcctaaaag

SEQ ID NO: 177 - DNA, 46 bp, Artificial sequence
  Primer
taatacgactcactatagggagatcgcttgggaggtggatttgaac

SEQ ID NO: 178 - DNA, 42 bp, Artificial sequence
  Primer
taatacgactcactatagggagagcgtgcgcagcgtggactc

SEQ ID NO: 179 - DNA, 49 bp, Artificial sequence
  Primer
taatacgactcactatagggagaggtcgctggccctggtaatggtggag

SEQ ID NO: 180 - DNA, 50 bp, Artificial sequence
  Primer
taatacgactcactatagggagagcggcgagtggagcaggggaaagtaga

SEQ ID NO: 181 - DNA, 48 bp, Artificial sequence
  Primer
taatacgactcactatagggagaggaggatcggttgaagctaaggata

SEQ ID NO: 182 - DNA, 48 bp, Artificial sequence
  Primer
taatacgactcactatagggagagaactggagctgatcttgcgtttca

SEQ ID NO: 183 - DNA, 48 bp, Artificial sequence
  Primer
taatacgactcactatagggagaaaacatcaacagctctgtggacagt

SEQ ID NO: 184 - DNA, 48 bp, Artificial sequence
  Primer
taatacgactcactatagggagactgcagctcgattagcacattatca

SEQ ID NO: 185 - DNA, 48 bp, Artificial sequence
  Primer
taatacgactcactatagggagaataatacggcatctggcagtcatag

SEQ ID NO: 186 - DNA, 48 bp, Artificial sequence
  Primer
taatacgactcactatagggagatccttggtaagaccttgagcatttg

SEQ ID NO: 187 - DNA, 48 bp, Artificial sequence
  Primer
taatacgactcactatagggagaggtcacaccagtctttgcgctctac

SEQ ID NO: 188 - DNA, 48 bp, Artificial sequence
  Primer
taatacgactcactatagggagaccatggcctgcactagattctgggt

SEQ ID NO: 189 - DNA, 48 bp, Artificial sequence
  Primer
taatacgactcactatagggagaaaaatggtttggatctgctggagga

SEQ ID NO: 190 - DNA, 48 bp, Artificial sequence
  Primer
taatacgactcactatagggagacaatccgaagagctgcgaaatgttg

SEQ ID NO: 191 - DNA, 48 bp, Artificial sequence
  Primer
taatacgactcactatagggagagtgccgatctgatgcagtgggagat

SEQ ID NO: 192 - DNA, 48 bp, Artificial sequence
  Primer
taatacgactcactatagggagatagctggctcagatcctcggtgttc

SEQ ID NO: 193 - DNA, 48 bp, Artificial sequence
  Primer
taatacgactcactatagggagacgggatgcaaaggacactggagatg

SEQ ID NO: 194 - DNA, 48 bp, Artificial sequence
  Primer
taatacgactcactatagggagacaacggcggtgctggcggtatgtta

SEQ ID NO: 195 - DNA, 50 bp, Artificial sequence
  Primer
taatacgactcactatagggagagagcgcaaaggagattaacagcaccct

SEQ ID NO: 196 - DNA, 48 bp, Artificial sequence
  Primer
taatacgactcactatagggagatcatcgccgaaagaaggagctcttg

SEQ ID NO: 197 - DNA, 48 bp, Artificial sequence
  Primer
taatacgactcactatagggagaagaagctgatgcagaccacacactc

SEQ ID NO: 198 - DNA, 48 bp, Artificial sequence
  Primer
taatacgactcactatagggagagcgcggcgttatttgcatggaatga

SEQ ID NO: 199 - DNA, 48 bp, Artificial sequence
  Primer
taatacgactcactatagggagatgaactctttctacaagacaaccag

SEQ ID NO: 200 - DNA, 48 bp, Artificial sequence
  Primer
taatacgactcactatagggagagcagactgttgtatgacatattcag

SEQ ID NO: 201 - DNA, 48 bp, Artificial sequence
  Primer
taatacgactcactatagggagacgatttcggattggctcaaaggata

SEQ ID NO: 202 - DNA, 48 bp, Artificial sequence
  Primer
taatacgactcactatagggagagaccagagagcaaagagagcattat

SEQ ID NO: 203 - DNA, 48 bp, Artificial sequence
  Primer
taatacgactcactatagggagagggtcacagctggcggcaaagggga

SEQ ID NO: 204 - DNA, 48 bp, Artificial sequence
  Primer
taatacgactcactatagggagacggagagttgcagcgcgtgggactg

SEQ ID NO: 205 - DNA, 43 bp, Artificial sequence
  Primer
gcttctaatacgactcactataggtgtacggcacacccgagta

SEQ ID NO: 206 - DNA, 43 bp, Artificial sequence
  Primer
gcttctaatacgactcactatagagacgatcccgtggattctg

SEQ ID NO: 207 - DNA, 48 bp, Artificial sequence
  Primer
taatacgactcactatagggagacaatctgaagatgggcaaccaacaa

SEQ ID NO: 208 - DNA, 48 bp, Artificial sequence
  Primer
taatacgactcactatagggagatctgatttagctgctgctcatccaa

SEQ ID NO: 209 - DNA, 45 bp, Artificial sequence
  Primer
gcttctaatacgactcactatagtcctgacctaactggtctctcc

SEQ ID NO: 210 - DNA, 44 bp, Artificial sequence
  Primer
gcttctaatacgactcactatagcatatccactccggttccata

SEQ ID NO: 211 - DNA, 48 bp, Artificial sequence
  Primer
taatacgactcactatagggagaaccagctgctaatgcgaattgagtg

SEQ ID NO: 212 - DNA, 48 bp, Artificial sequence
  Primer
taatacgactcactatagggagagcggaaaagtatgcggaatatctgg

SEQ ID NO: 213 - DNA, 48 bp, Artificial sequence
  Primer
taatacgactcactatagggagacacatcaggcggcacagcaggaaca

SEQ ID NO: 214 - DNA, 48 bp, Artificial sequence
  Primer
taatacgactcactatagggagaagctgatgacgcagaggacgagatg

SEQ ID NO: 215 - DNA, 44 bp, Artificial sequence
  Primer
gcttctaatacgactcactatagcctcatctggacacgtagagc

SEQ ID NO: 216 - DNA, 43 bp, Artificial sequence
  Primer
gcttctaatacgactcactatagggagctcctgtcctgttctg

SEQ ID NO: 217 - DNA, 48 bp, Artificial sequence
  Primer
taatacgactcactatagggagaagagattcgatgcggcagtcatcca

SEQ ID NO: 218 - DNA, 48 bp, Artificial sequence
  Primer
taatacgactcactatagggagatgactgaactctagagccgcctcat

SEQ ID NO: 219 - DNA, 48 bp, Artificial sequence
  Primer
taatacgactcactatagggagagacgaagatatcggtatacgagtgg

SEQ ID NO: 220 - DNA, 48 bp, Artificial sequence
  Primer
taatacgactcactatagggagatggatagagccaggcttgtttctga

SEQ ID NO: 221 - DNA, 48 bp, Artificial sequence
  Primer
taatacgactcactatagggagactgaccaaacagatctgctaccaga

SEQ ID NO: 222 - DNA, 48 bp, Artificial sequence
  Primer
taatacgactcactatagggagatgtccaggcacttcttgagaaagtc

SEQ ID NO: 223 - DNA, 48 bp, Artificial sequence
  Primer
taatacgactcactatagggagaggcagaaggcgctgaaggaaatcat

SEQ ID NO: 224 - DNA, 48 bp, Artificial sequence
  Primer
taatacgactcactatagggagaggaagattgagcacgctcttgttgg

SEQ ID NO: 225 - DNA, 48 bp, Artificial sequence
  Primer
taatacgactcactatagggagaggcctggcttacgatgggatagtaa

SEQ ID NO: 226 - DNA, 48 bp, Artificial sequence
  Primer
taatacgactcactatagggagagtgcctaggtcgctgatgaatctct

SEQ ID NO: 227 - DNA, 48 bp, Artificial sequence
  Primer
taatacgactcactatagggagaaagccggatgcagacttcattaccc

SEQ ID NO: 228 - DNA, 48 bp, Artificial sequence
  Primer
taatacgactcactatagggagaaccaccttcagggcgaactcatttc

SEQ ID NO: 229 - DNA, 48 bp, Artificial sequence
  Primer
taatacgactcactatagggagataacaaacagcaacagcaacataac

SEQ ID NO: 230 - DNA, 44 bp, Artificial sequence
  Primer
taatacgactcactatagggagacgtcttcgaggtggagggtaa

SEQ ID NO: 231 - DNA, 48 bp, Artificial sequence
  Primer
taatacgactcactatagggagacgcaccggaggtcatagacgaagtg

SEQ ID NO: 232 - DNA, 49 bp, Artificial sequence
  Primer
taatacgactcactatagggagatggccgctggacgatcctcgctgaga

SEQ ID NO: 233 - DNA, 48 bp, Artificial sequence
  Primer
taatacgactcactatagggagaaacaggaaattccacgaatagaagg

SEQ ID NO: 234 - DNA, 48 bp, Artificial sequence
  Primer
taatacgactcactatagggagaagcatttagagccggtaacgtgtat

SEQ ID NO: 235 - DNA, 48 bp, Artificial sequence
  Primer
taatacgactcactatagggagaccagcatgccgttcgaaatgaaaca

SEQ ID NO: 236 - DNA, 48 bp, Artificial sequence
  Primer
taatacgactcactatagggagacacctgctgggcaatttgcttgata

SEQ ID NO: 237 - DNA, 48 bp, Artificial sequence
  Primer
taatacgactcactatagggagaaaattgcgctggatgctggtttggg

SEQ ID NO: 238 - DNA, 48 bp, Artificial sequence
  Primer
taatacgactcactatagggagacagtggtctgatctgggtacttgag

SEQ ID NO: 239 - DNA, 48 bp, Artificial sequence
  Primer
taatacgactcactatagggagaacgtggtcgctgggtggaagtatgg

SEQ ID NO: 240 - DNA, 53 bp, Artificial sequence
  Primer
taatacgactcactatagggagaatgaaaaatggccggtaaaacgctggaacg

SEQ ID NO: 241 - DNA, 49 bp, Artificial sequence
  Primer
taatacgactcactatagggagatgcaattcctcgaagatcaaagtgaa

SEQ ID NO: 242 - DNA, 48 bp, Artificial sequence
  Primer
taatacgactcactatagggagaggactatcaaagtggagtgctaatc

SEQ ID NO: 243 - DNA, 48 bp, Artificial sequence
  Primer
taatacgactcactatagggagaagaaccgaaaggtgcagctggtgga

SEQ ID NO: 244 - DNA, 48 bp, Artificial sequence
  Primer
taatacgactcactatagggagactctcacgcaattcaagaggagagg

SEQ ID NO: 245 - DNA, 48 bp, Artificial sequence
  Primer
taatacgactcactatagggagactctggagcatcggggtcatcctct

SEQ ID NO: 246 - DNA, 48 bp, Artificial sequence
  Primer
taatacgactcactatagggagagcctgccgttgacgttcagccaaca

SEQ ID NO: 247 - DNA, 48 bp, Artificial sequence
  Primer
taatacgactcactatagggagaattcttgaggagcatggcatcatac

SEQ ID NO: 248 - DNA, 48 bp, Artificial sequence
  Primer
taatacgactcactatagggagagaaggttttcgagagtatcacttgg

SEQ ID NO: 249 - DNA, 48 bp, Artificial sequence
  Primer
taatacgactcactatagggagagacgctggacatgggtaatatgttc

SEQ ID NO: 250 - DNA, 48 bp, Artificial sequence
  Primer
taatacgactcactatagggagatttgatccagggagagcagcaggtt

SEQ ID NO: 251 - DNA, 47 bp, Artificial sequence
  Primer
taatacgactcactatagggagagtcaaggcagcacaccatcatcat

SEQ ID NO: 252 - DNA, 43 bp, Artificial sequence
  Primer
taatacgactcactatagggagatgcagcccgccgacacagta

SEQ ID NO: 253 - DNA, 48 bp, Artificial sequence
  Primer
taatacgactcactatagggagaaaacagaagatctgccacggggaca

SEQ ID NO: 254 - DNA, 48 bp, Artificial sequence
  Primer
taatacgactcactatagggagatccaagtaatcctcggcgctctttc

SEQ ID NO: 255 - DNA, 48 bp, Artificial sequence
  Primer
taatacgactcactatagggagaacaaccactacaaatgcctcagtcc

SEQ ID NO: 256 - DNA, 48 bp, Artificial sequence
  Primer
taatacgactcactatagggagatgatggcggactgcggtttagattg

SEQ ID NO: 257 - DNA, 48 bp, Artificial sequence
  Primer
taatacgactcactatagggagaatctacgaggccaagcacatggggt

SEQ ID NO: 258 - DNA, 48 bp, Artificial sequence
  Primer
taatacgactcactatagggagaccgccgggactgcactttacaacaa

SEQ ID NO: 259 - DNA, 48 bp, Artificial sequence
  Primer
taatacgactcactatagggagacgtcaccatggcaaggaaaagaact

SEQ ID NO: 260 - DNA, 48 bp, Artificial sequence
  Primer
taatacgactcactatagggagagcgtggacatcttcttttcggagat

SEQ ID NO: 261 - DNA, 48 bp, Artificial sequence
  Primer
taatacgactcactatagggagacaatcaagacgattatcagttggtc

SEQ ID NO: 262 - DNA, 48 bp, Artificial sequence
  Primer
taatacgactcactatagggagaccagtaattcgggacctttaaagta

SEQ ID NO: 263 - DNA, 43 bp, Artificial sequence
  Primer
taatacgactcactatagggagaattaggccgtggaaagtatt

SEQ ID NO: 264 - DNA, 43 bp, Artificial sequence
  Primer
taatacgactcactatagggagacgaagccacacgaacattat

SEQ ID NO: 265 - DNA, 48 bp, Artificial sequence
  Primer
taatacgactcactatagggagacggataacttcctcatgggtcttgg

SEQ ID NO: 266 - DNA, 48 bp, Artificial sequence
  Primer
taatacgactcactatagggagaaggtccgccaaacttaagcaggttc

SEQ ID NO: 267 - DNA, 48 bp, Artificial sequence
  Primer
taatacgactcactatagggagaccggacattgtgtgccaggactatg

SEQ ID NO: 268 - DNA, 48 bp, Artificial sequence
  Primer
taatacgactcactatagggagacgcacaaatgcagctcaccaaatac

SEQ ID NO: 269 - DNA, 47 bp, Artificial sequence
  Primer
taatacgactcactatagggagaaccaccgacaccaaacatacatac

SEQ ID NO: 270 - DNA, 47 bp, Artificial sequence
  Primer
taatacgactcactatagggagaaattgccttttccacaccatagtt

SEQ ID NO: 271 - DNA, 47 bp, Artificial sequence
  Primer
taatacgactcactatagggagagaatttcacactaagccatacaag

SEQ ID NO: 272 - DNA, 41 bp, Artificial sequence
  Primer
taatacgactcactatagggagactctcccaagccatccag

SEQ ID NO: 273 - DNA, 48 bp, Artificial sequence
  Primer
taatacgactcactatagggagagaccggcgatcaatgtgtcacacag

SEQ ID NO: 274 - DNA, 48 bp, Artificial sequence
  Primer
taatacgactcactatagggagaactggaactttccgtggcaaggagg

SEQ ID NO: 275 - DNA, 48 bp, Artificial sequence
  Primer
taatacgactcactatagggagaggcagcacaaataccgctacaggga

SEQ ID NO: 276 - DNA, 48 bp, Artificial sequence
  Primer
taatacgactcactatagggagattggtccagcgggtatggctcatag

SEQ ID NO: 277 - DNA, 48 bp, Artificial sequence
  Primer
taatacgactcactatagggagacacgaggagtacgacgacgatgact

SEQ ID NO: 278 - DNA, 48 bp, Artificial sequence
  Primer
taatacgactcactatagggagatcagctcttggagacggcggttgaa

SEQ ID NO: 279 - DNA, 48 bp, Artificial sequence
  Primer
taatacgactcactatagggagacgggaatcgagcacagcattgagta

SEQ ID NO: 280 - DNA, 48 bp, Artificial sequence
  Primer
taatacgactcactatagggagacttcgtcctgtgcttacacttccac

SEQ ID NO: 281 - DNA, 45 bp, Artificial sequence
  Primer
gcttctaatacgactcactatagccttgacgaagagtcctatgtg

SEQ ID NO: 282 - DNA, 45 bp, Artificial sequence
  Primer
gcttctaatacgactcactatagccaagtaattggtaagctcgaa

SEQ ID NO: 283 - DNA, 48 bp, Artificial sequence
  Primer
taatacgactcactatagggagagctgtccgacgaggatctggagaag

SEQ ID NO: 284 - DNA, 48 bp, Artificial sequence
  Primer
taatacgactcactatagggagaagctacgggtgcccacaaaggagtt

SEQ ID NO: 285 - DNA, 48 bp, Artificial sequence
  Primer
taatacgactcactatagggagatcgctgttccattcaggccaccaag

SEQ ID NO: 286 - DNA, 48 bp, Artificial sequence
  Primer
taatacgactcactatagggagaaggcacctggtccgattggctgatg

SEQ ID NO: 287 - DNA, 48 bp, Artificial sequence
  Primer
taatacgactcactatagggagaggccattaaggagctgctcaagtcc

SEQ ID NO: 288 - DNA, 48 bp, Artificial sequence
  Primer
taatacgactcactatagggagactggccaaaggtcagcagttcccaa

SEQ ID NO: 289 - DNA, 48 bp, Artificial sequence
  Primer
taatacgactcactatagggagactacaattcggaggaatacttggac

SEQ ID NO: 290 - DNA, 48 bp, Artificial sequence
  Primer
taatacgactcactatagggagatgacgatgttgctgtgtttcagttc

SEQ ID NO: 291 - DNA, 48 bp, Artificial sequence
  Primer
taatacgactcactatagggagaggtaacgacatacactgtgcagata

SEQ ID NO: 292 - DNA, 48 bp, Artificial sequence
  Primer
taatacgactcactatagggagactgaaccaacggattgaagagtttg

SEQ ID NO: 293 - DNA, 48 bp, Artificial sequence
  Primer
taatacgactcactatagggagatcatccacgtgcatatgccgaacaa

SEQ ID NO: 294 - DNA, 48 bp, Artificial sequence
  Primer
taatacgactcactatagggagagaaataatgacgaatgcccagacag

SEQ ID NO: 295 - DNA, 48 bp, Artificial sequence
  Primer
taatacgactcactatagggagacgttcagcagaagtgtggtcaggtc

SEQ ID NO: 296 - DNA, 48 bp, Artificial sequence
  Primer
taatacgactcactatagggagaccgtccgctggcagttgtacaggat

SEQ ID NO: 297 - DNA, 48 bp, Artificial sequence
  Primer
taatacgactcactatagggagagaggagcagagacagatacacacac

SEQ ID NO: 298 - DNA, 48 bp, Artificial sequence
  Primer
taatacgactcactatagggagaaaggcttcggggatcctggttcaat

SEQ ID NO: 299 - DNA, 48 bp, Artificial sequence
  Primer
taatacgactcactatagggagacaatagcaatcacagtgcctcacag

SEQ ID NO: 300 - DNA, 48 bp, Artificial sequence
  Primer
taatacgactcactatagggagagcacgcaatagcagtgatccttcat

SEQ ID NO: 301 - DNA, 48 bp, Artificial sequence
  Primer
taatacgactcactatagggagatacaaggccaaactgaagtccacca

SEQ ID NO: 302 - DNA, 48 bp, Artificial sequence
  Primer
taatacgactcactatagggagacatcagtatgccataggaccacaca

SEQ ID NO: 303 - DNA, 48 bp, Artificial sequence
  Primer
taatacgactcactatagggagaattaagcgcatcaacctggagaagt

SEQ ID NO: 304 - DNA, 48 bp, Artificial sequence
  Primer
taatacgactcactatagggagaccaaatgtccgccttaaagtcatag

SEQ ID NO: 305 - DNA, 50 bp, Artificial sequence
  Primer
taatacgactcactatagggagaacccgccaatctgtctagcaataatgt

SEQ ID NO: 306 - DNA, 47 bp, Artificial sequence
  Primer
taatacgactcactatagggagacttccttcagtaccgtggcaatgg

SEQ ID NO: 307 - DNA, 48 bp, Artificial sequence
  Primer
taatacgactcactatagggagacggggctgacggacaatttcttgat

SEQ ID NO: 308 - DNA, 48 bp, Artificial sequence
  Primer
taatacgactcactatagggagactgttggtggtgttgctgctgatgt

SEQ ID NO: 309 - DNA, 48 bp, Artificial sequence
  Primer
taatacgactcactatagggagagcatatcctggacgcagctgttgtg

SEQ ID NO: 310 - DNA, 48 bp, Artificial sequence
  Primer
taatacgactcactatagggagaactggcgtacggttggagcgactat

SEQ ID NO: 311 - DNA, 48 bp, Artificial sequence
  Primer
taatacgactcactatagggagaagagcgacgaggtgctggaacacac

SEQ ID NO: 312 - DNA, 48 bp, Artificial sequence
  Primer
taatacgactcactatagggagatcgcgtaatcggggcagttcactgg

SEQ ID NO: 313 - DNA, 48 bp, Artificial sequence
  Primer
taatacgactcactatagggagagcaacaaacacaggaaaggctgaag

SEQ ID NO: 314 - DNA, 48 bp, Artificial sequence
  Primer
taatacgactcactatagggagaggatatgaggtccgatctggtttga

SEQ ID NO: 315 - DNA, 45 bp, Artificial sequence
  Primer
gcttctaatacgactcactataggcatataccatatcgggagcat

SEQ ID NO: 316 - DNA, 45 bp, Artificial sequence
  Primer
gcttctaatacgactcactatagctcgtttcgtatcaccgatttt

SEQ ID NO: 317 - DNA, 54 bp, Artificial sequence
  Primer
taatacgactcactatagggagaacttgttactaatcgatttccgttctctttc

SEQ ID NO: 318 - DNA, 52 bp, Artificial sequence
  Primer
taatacgactcactatagggagacgtagtgcgttctggatcctctgttattt

SEQ ID NO: 319 - DNA, 43 bp, Artificial sequence
  Primer
gcttctaatacgactcactatagagctctcaaactcccggaac

SEQ ID NO: 320 - DNA, 44 bp, Artificial sequence
  Primer
gcttctaatacgactcactatagcagcgacatcaatcacaagaa

SEQ ID NO: 321 - DNA, 48 bp, Artificial sequence
  Primer
taatacgactcactatagggagacagcgacgatgacttcaatgtcaga

SEQ ID NO: 322 - DNA, 48 bp, Artificial sequence
  Primer
taatacgactcactatagggagatgggtcctttaagcacgatatcctc

SEQ ID NO: 323 - DNA, 42 bp, Artificial sequence
  Primer
taatacgactcactatagggagaaaccacttcgcagcagaga

SEQ ID NO: 324 - DNA, 42 bp, Artificial sequence
  Primer
taatacgactcactatagggagaatcccgatggtgaaggtgt

SEQ ID NO: 325 - DNA, 47 bp, Artificial sequence
  Primer
taatacgactcactatagggagagaagcttactgattttgggttgag

SEQ ID NO: 326 - DNA, 47 bp, Artificial sequence
  Primer
taatacgactcactatagggagacgtttgtagtgcaggtatgagtaa

SEQ ID NO: 327 - DNA, 48 bp, Artificial sequence
  Primer
taatacgactcactatagggagagcgatagcaagatatctggtgtttc

SEQ ID NO: 328 - DNA, 48 bp, Artificial sequence
  Primer
taatacgactcactatagggagagcttaggttgtccacattcttctca

SEQ ID NO: 329 - DNA, 48 bp, Artificial sequence
  Primer
taatacgactcactatagggagagcgtgattatgcgtccaacgtgata

SEQ ID NO: 330 - DNA, 48 bp, Artificial sequence
  Primer
taatacgactcactatagggagactgtcagccagcatctgaagattga

SEQ ID NO: 331 - DNA, 48 bp, Artificial sequence
  Primer
taatacgactcactatagggagaacaggcctcgcttagcacgctgaat

SEQ ID NO: 332 - DNA, 48 bp, Artificial sequence
  Primer
taatacgactcactatagggagaggaaagctgcctgaatagcggagag

SEQ ID NO: 333 - DNA, 48 bp, Artificial sequence
  Primer
taatacgactcactatagggagagagctgatgtccatgtgctttgaca

SEQ ID NO: 334 - DNA, 48 bp, Artificial sequence
  Primer
taatacgactcactatagggagactgatggttctttgtgaggcacttg

SEQ ID NO: 335 - DNA, 48 bp, Artificial sequence
  Primer
taatacgactcactatagggagaagattcgctatccggagcacaagga

SEQ ID NO: 336 - DNA, 48 bp, Artificial sequence
  Primer
taatacgactcactatagggagacgtgtggaaagactcgcacatagac

SEQ ID NO: 337 - DNA, 48 bp, Artificial sequence
  Primer
taatacgactcactatagggagatctgcgagtggtgcgaagtcttcac

SEQ ID NO: 338 - DNA, 48 bp, Artificial sequence
  Primer
taatacgactcactatagggagaccgcaccaagctggcaatgtcatca

SEQ ID NO: 339 - DNA, 48 bp, Artificial sequence
  Primer
taatacgactcactatagggagatgttcaaagaggagctgcgcaaggg

SEQ ID NO: 340 - DNA, 48 bp, Artificial sequence
  Primer
taatacgactcactatagggagactccatgcgccggatcttgctgtag

SEQ ID NO: 341 - DNA, 48 bp, Artificial sequence
  Primer
taatacgactcactatagggagagggcaaaccatatacaagcttactg

SEQ ID NO: 342 - DNA, 48 bp, Artificial sequence
  Primer
taatacgactcactatagggagaaactagtccgattggtgaagaacac

SEQ ID NO: 343 - DNA, 48 bp, Artificial sequence
  Primer
taatacgactcactatagggagagataaaagagcgcagcgatgtgaat

SEQ ID NO: 344 - DNA, 48 bp, Artificial sequence
  Primer
taatacgactcactatagggagaggcgaattgcatgctccaataatag

SEQ ID NO: 345 - DNA, 49 bp, Artificial sequence
  Primer
taatacgactcactatagggagatcagtgcgagggtaggtagaaatgtt

SEQ ID NO: 346 - DNA, 45 bp, Artificial sequence
  Primer
taatacgactcactatagggagagtccgcactcgtcgcagaatgt

SEQ ID NO: 347 - DNA, 48 bp, Artificial sequence
  Primer
taatacgactcactatagggagaactcctgatgggcagactgtaatgg

SEQ ID NO: 348 - DNA, 48 bp, Artificial sequence
  Primer
taatacgactcactatagggagacactgggtgacttgtcaagttggtg

SEQ ID NO: 349 - DNA, 44 bp, Artificial sequence
  Primer
gcttctaatacgactcactatagtctgcaatggtgcgataattt

SEQ ID NO: 350 - DNA, 44 bp, Artificial sequence
  Primer
gcttctaatacgactcactatagcctttctgcctcttcgatagc

SEQ ID NO: 351 - DNA, 44 bp, Artificial sequence
  Primer
gcttctaatacgactcactatagtcaggagaatgttcaggttcc

SEQ ID NO: 352 - DNA, 43 bp, Artificial sequence
  Primer
gcttctaatacgactcactatagatattccttggccagctcag

SEQ ID NO: 353 - DNA, 44 bp, Artificial sequence
  Primer
gcttctaatacgactcactatagtcgagtgtaacggaaatcgac

SEQ ID NO: 354 - DNA, 44 bp, Artificial sequence
  Primer
gcttctaatacgactcactataggaatcggagtgtgtgtgtgtg

SEQ ID NO: 355 - DNA, 48 bp, Artificial sequence
  Primer
taatacgactcactatagggagaatgccagggtgatcttccgacagtt

SEQ ID NO: 356 - DNA, 48 bp, Artificial sequence
  Primer
taatacgactcactatagggagaaaacggacgcaatcggtcggattca

SEQ ID NO: 357 - DNA, 48 bp, Artificial sequence
  Primer
taatacgactcactatagggagattggtgtctgctattgaatactgtc

SEQ ID NO: 358 - DNA, 48 bp, Artificial sequence
  Primer
taatacgactcactatagggagacgatttacatcatgcagatccatag

SEQ ID NO: 359 - DNA, 48 bp, Artificial sequence
  Primer
taatacgactcactatagggagaatgggctacctgcacgcaagggaga

SEQ ID NO: 360 - DNA, 48 bp, Artificial sequence
  Primer
taatacgactcactatagggagaaaaggttgacggggtgggagggact

SEQ ID NO: 361 - DNA, 48 bp, Artificial sequence
  Primer
taatacgactcactatagggagaggacctttgacatcgatgcagatag

SEQ ID NO: 362 - DNA, 48 bp, Artificial sequence
  Primer
taatacgactcactatagggagaggcatcaatggttttggcaatggag

SEQ ID NO: 363 - DNA, 44 bp, Artificial sequence
  Primer
gcttctaatacgactcactatagcattgtcggggtcaactactg

SEQ ID NO: 364 - DNA, 44 bp, Artificial sequence
  Primer
gcttctaatacgactcactataggtccttgggtatctcgaccag

SEQ ID NO: 365 - DNA, 48 bp, Artificial sequence
  Primer
taatacgactcactatagggagaaagatgatgaggatggagagaatga

SEQ ID NO: 366 - DNA, 48 bp, Artificial sequence
  Primer
taatacgactcactatagggagagtgttgtagtcataactggtgtttg

SEQ ID NO: 367 - DNA, 48 bp, Artificial sequence
  Primer
taatacgactcactatagggagactgcgaacttaccaatccagtttat

SEQ ID NO: 368 - DNA, 48 bp, Artificial sequence
  Primer
taatacgactcactatagggagatcgctaagtagtttgttgctgatga

SEQ ID NO: 369 - DNA, 48 bp, Artificial sequence
  Primer
taatacgactcactatagggagaccactgacggacgactacgtgacct

SEQ ID NO: 370 - DNA, 48 bp, Artificial sequence
  Primer
taatacgactcactatagggagagcagcgtgtaactggtgaatgtctc

SEQ ID NO: 371 - DNA, 48 bp, Artificial sequence
  Primer
taatacgactcactatagggagagccagatccaattcgctgcggagtt

SEQ ID NO: 372 - DNA, 48 bp, Artificial sequence
  Primer
taatacgactcactatagggagattggtgatggtgcggatgcggatga

SEQ ID NO: 373 - DNA, 49 bp, Artificial sequence
  Primer
taatacgactcactatagggagaccggtgcccatccctccctgctctat

SEQ ID NO: 374 - DNA, 48 bp, Artificial sequence
  Primer
taatacgactcactatagggagacccgctggaactggatgccctggac

SEQ ID NO: 375 - DNA, 48 bp, Artificial sequence
  Primer
taatacgactcactatagggagattttgagaagccattgaaggaggag

SEQ ID NO: 376 - DNA, 48 bp, Artificial sequence
  Primer
taatacgactcactatagggagagtacctggagacattcatcggttat

SEQ ID NO: 377 - DNA, 43 bp, Artificial sequence
  Primer
taatacgactcactatagggagattacctgcgtgccaagttcg

SEQ ID NO: 378 - DNA, 48 bp, Artificial sequence
  Primer
taatacgactcactatagggagagctctgctcgcgtcgtaatcgtatg

SEQ ID NO: 379 - DNA, 44 bp, Artificial sequence
  Primer
gcttctaatacgactcactatagcattttcagtgtcgagccatt

SEQ ID NO: 380 - DNA, 44 bp, Artificial sequence
  Primer
gcttctaatacgactcactatagcaccgtagcatcgtagtggtt

SEQ ID NO: 381 - DNA, 48 bp, Artificial sequence
  Primer
taatacgactcactatagggagaacttcagacgatctcgaggatgagg

SEQ ID NO: 382 - DNA, 48 bp, Artificial sequence
  Primer
taatacgactcactatagggagacgcatccttggtcttggcaatagag

SEQ ID NO: 383 - DNA, 48 bp, Artificial sequence
  Primer
taatacgactcactatagggagagcaccaacagcctgggcagcctgaa

SEQ ID NO: 384 - DNA, 48 bp, Artificial sequence
  Primer
taatacgactcactatagggagatccggcgggctgagatgggaagaga

SEQ ID NO: 385 - DNA, 42 bp, Artificial sequence
  Primer
taatacgactcactatagggagatagcggcggcgttatggat

SEQ ID NO: 386 - DNA, 45 bp, Artificial sequence
  Primer
taatacgactcactatagggagagctgggcacgacgtttcttttt

SEQ ID NO: 387 - DNA, 48 bp, Artificial sequence
  Primer
taatacgactcactatagggagatcaaacattgccgtcaacgaggatt

SEQ ID NO: 388 - DNA, 48 bp, Artificial sequence
  Primer
taatacgactcactatagggagacaaatccatatcctcgaagctgtga

SEQ ID NO: 389 - DNA, 48 bp, Artificial sequence; identical to SEQ ID NO: 378
  Primer

SEQ ID NO: 390 - DNA, 49 bp, Artificial sequence
  Primer
taatacgactcactatagggagatgctaccaccgcttccgctaccacta

SEQ ID NO: 391 - DNA, 44 bp, Artificial sequence
  Primer
gcttctaatacgactcactatagcacgacgacaaacacagacac

SEQ ID NO: 392 - DNA, 44 bp, Artificial sequence
  Primer
gcttctaatacgactcactatagttgcatgtacagtcggtcgta

SEQ ID NO: 393 - DNA, 48 bp, Artificial sequence
  Primer
taatacgactcactatagggagacacagaacacgcaatcaggggaaac

SEQ ID NO: 394 - DNA, 48 bp, Artificial sequence
  Primer
taatacgactcactatagggagaagatgggtaacgaccgctggtattg

SEQ ID NO: 395 - DNA, 45 bp, Artificial sequence
  Primer
gcttctaatacgactcactatagactacatgagtccggagttggt

SEQ ID NO: 396 - DNA, 45 bp, Artificial sequence
  Primer
gcttctaatacgactcactatagttcacttcgcaaatctggagta

SEQ ID NO: 397 - DNA, 48 bp, Artificial sequence
  Primer
taatacgactcactatagggagaagctactcgggcaagtccacaaatg

SEQ ID NO: 398 - DNA, 48 bp, Artificial sequence
  Primer
taatacgactcactatagggagacagccaaaactttgcgaacggtctc

SEQ ID NO: 399 - DNA, 48 bp, Artificial sequence
  Primer
taatacgactcactatagggagagccgaccacatcaaggtgttcctgt

SEQ ID NO: 400 - DNA, 48 bp, Artificial sequence
  Primer
taatacgactcactatagggagaagacgagcatctggcagagcaagtg

SEQ ID NO: 401 - DNA, 48 bp, Artificial sequence
  Primer
taatacgactcactatagggagaagatctactagtcgctgttaagatg

SEQ ID NO: 402 - DNA, 48 bp, Artificial sequence
  Primer
taatacgactcactatagggagaaagcgagaacttgttgtacagtatg

SEQ ID NO: 403 - DNA, 48 bp, Artificial sequence
  Primer
taatacgactcactatagggagacaagccgacaattcagtgggacaag

SEQ ID NO: 404 - DNA, 48 bp, Artificial sequence
  Primer
taatacgactcactatagggagactgcaggctgtgtcatcggatttct

SEQ ID NO: 405 - DNA, 48 bp, Artificial sequence
  Primer
taatacgactcactatagggagagcgcaaaatggccaaattctacaag

SEQ ID NO: 406 - DNA, 48 bp, Artificial sequence
  Primer
taatacgactcactatagggagaaaatccaggatgcgaagctcacagt

SEQ ID NO: 407 - DNA, 46 bp, Artificial sequence
  Primer
taatacgactcactatagggagatgagactacgaggcactgaaaat

SEQ ID NO: 408 - DNA, 46 bp, Artificial sequence
  Primer
taatacgactcactatagggagagtctgcgcacatacgggataaac

SEQ ID NO: 409 - DNA, 48 bp, Artificial sequence
  Primer
taatacgactcactatagggagatcttggagaaactgcgcaccattgt

SEQ ID NO: 410 - DNA, 48 bp, Artificial sequence
  Primer
taatacgactcactatagggagactaccatcgttgtgcgtttggattg

SEQ ID NO: 411 - DNA, 43 bp, Artificial sequence
  Primer
gcttctaatacgactcactatagaccagtaccgcccaagaaat

SEQ ID NO: 412 - DNA, 44 bp, Artificial sequence
  Primer
gcttctaatacgactcactataggttcccttgggtcatctgaat

SEQ ID NO: 413 - DNA, 49 bp, Artificial sequence
  Primer
taatacgactcactatagggagatggcagcaactttaagcgacagaaca

SEQ ID NO: 414 - DNA, 46 bp, Artificial sequence
  Primer
taatacgactcactatagggagagtggtggagcgacgtggaatgat

SEQ ID NO: 415 - DNA, 48 bp, Artificial sequence
  Primer
taatacgactcactatagggagacaagcagagcaagcgctacggtgaa

SEQ ID NO: 416 - DNA, 48 bp, Artificial sequence
  Primer
taatacgactcactatagggagatcctcacgccgcttagacgctgaaa

SEQ ID NO: 417 - DNA, 49 bp, Artificial sequence
  Primer
taatacgactcactatagggagaatgtggttcgcgatgcttacgagaat

SEQ ID NO: 418 - DNA, 48 bp, Artificial sequence
  Primer
taatacgactcactatagggagaatgattgcatctgttccgaatcctt

SEQ ID NO: 419 - DNA, 48 bp, Artificial sequence
  Primer
taatacgactcactatagggagacaccgcttgtagtcacgactttcat

SEQ ID NO: 420 - DNA, 48 bp, Artificial sequence
  Primer
taatacgactcactatagggagagcatctggatgtagaggtacacctt

SEQ ID NO: 421 - DNA, 48 bp, Artificial sequence
  Primer
taatacgactcactatagggagatcttcgactatctgacctctgtggt

SEQ ID NO: 422 - DNA, 48 bp, Artificial sequence
  Primer
taatacgactcactatagggagacttgacggttatacgttgcgaagga

SEQ ID NO: 423 - DNA, 48 bp, Artificial sequence
  Primer
taatacgactcactatagggagaactctgcatgtggaggagatctttg

SEQ ID NO: 424 - DNA, 48 bp, Artificial sequence
  Primer
taatacgactcactatagggagagcattatcaaactgcgctgcacttc

SEQ ID NO: 425 - DNA, 48 bp, Artificial sequence
  Primer
taatacgactcactatagggagaatgacgatgaggaaagcgaggagag

SEQ ID NO: 426 - DNA, 48 bp, Artificial sequence
  Primer
taatacgactcactatagggagacgggataatagttgggcaggggaat

SEQ ID NO: 427 - DNA, 48 bp, Artificial sequence
  Primer
taatacgactcactatagggagaacgtctggtctggtcacactgctac

SEQ ID NO: 428 - DNA, 53 bp, Artificial sequence
  Primer
taatacgactcactatagggagactgttgttgctgctgctgctgctcctgata

SEQ ID NO: 429 - DNA, 48 bp, Artificial sequence
  Primer
taatacgactcactatagggagaagccagggcgaagcagaaattatgg

SEQ ID NO: 430 - DNA, 48 bp, Artificial sequence
  Primer
taatacgactcactatagggagatcggtcatgtttgtggctggagaag

SEQ ID NO: 431 - DNA, 48 bp, Artificial sequence
  Primer
taatacgactcactatagggagacgacctcaagcccgagaacatcctg

SEQ ID NO: 432 - DNA, 48 bp, Artificial sequence
  Primer
taatacgactcactatagggagacaccaggtcctcggcgtccttatca

SEQ ID NO: 433 - DNA, 48 bp, Artificial sequence
  Primer
taatacgactcactatagggagacgcgacctcaagcccgagaacatcc

SEQ ID NO: 434 - DNA, 48 bp, Artificial sequence
  Primer
taatacgactcactatagggagagcaccaggtcctcggcgtccttatc

SEQ ID NO: 435 - DNA, 48 bp, Artificial sequence
  Primer
taatacgactcactatagggagaagaaggagaaccactttccggacat

SEQ ID NO: 436 - DNA, 48 bp, Artificial sequence
  Primer
taatacgactcactatagggagactccagaaagaagtccatccagaac

SEQ ID NO: 437 - DNA, 48 bp, Artificial sequence
  Primer
taatacgactcactatagggagatcgctgcgctaccacttcaaggaca

SEQ ID NO: 438 - DNA, 48 bp, Artificial sequence
  Primer
taatacgactcactatagggagacaggttgcgcagtaggtccttcaga

SEQ ID NO: 439 - DNA, 48 bp, Artificial sequence
  Primer
taatacgactcactatagggagacaacggaagtttcggcactgtgatg

SEQ ID NO: 440 - DNA, 48 bp, Artificial sequence
  Primer
taatacgactcactatagggagaccaccagtccaccgatttgttgtag

SEQ ID NO: 441 - DNA, 49 bp, Artificial sequence
  Primer
taatacgactcactatagggagagcccgcttctgcacgcctttgtcatc

SEQ ID NO: 442 - DNA, 54 bp, Artificial sequence
  Primer
taatacgactcactatagggagaactcgtcgtcgtcctcctcgtcatcggtttc

SEQ ID NO: 443 - DNA, 48 bp, Artificial sequence
  Primer
taatacgactcactatagggagatggaccgtttgttctttgtaatgga

SEQ ID NO: 444 - DNA, 48 bp, Artificial sequence
  Primer
taatacgactcactatagggagagcttatttggctgcttagttaggaa

SEQ ID NO: 445 - DNA, 48 bp, Artificial sequence
  Primer
taatacgactcactatagggagacacctttcctggtccaattacactc

SEQ ID NO: 446 - DNA, 48 bp, Artificial sequence
  Primer
taatacgactcactatagggagactttgctcaggctctttggatagga

SEQ ID NO: 447 - DNA, 48 bp, Artificial sequence
  Primer
taatacgactcactatagggagacgaagcagatggccgagatactcag

SEQ ID NO: 448 - DNA, 48 bp, Artificial sequence
  Primer
taatacgactcactatagggagaaacgcagtcaggaagggatggttgg

SEQ ID NO: 449 - DNA, 43 bp, Artificial sequence
  Primer
taatacgactcactatagggagaagcccgagaagcccgtgact

SEQ ID NO: 450 - DNA, 43 bp, Artificial sequence
  Primer
taatacgactcactatagggagatgcgttcgatgagcgtggag

SEQ ID NO: 451 - DNA, 48 bp, Artificial sequence
  Primer
taatacgactcactatagggagacagacgttctggagctggagttcta

SEQ ID NO: 452 - DNA, 48 bp, Artificial sequence
  Primer
taatacgactcactatagggagaggcaaagatatccacgcgatcctga

SEQ ID NO: 453 - DNA, 48 bp, Artificial sequence
  Primer
taatacgactcactatagggagaacgttagcgaggatctgcacaagta

SEQ ID NO: 454 - DNA, 48 bp, Artificial sequence
  Primer
taatacgactcactatagggagagcttaccacctttgatgctgtatcc

SEQ ID NO: 455 - DNA, 45 bp, Artificial sequence
  Primer
gcttctaatacgactcactatagggaactgggtgactctaagctg

SEQ ID NO: 456 - DNA, 45 bp, Artificial sequence
  Primer
gcttctaatacgactcactatagactcgagtcccactaccatgtc

SEQ ID NO: 457 - DNA, 48 bp, Artificial sequence
  Primer
taatacgactcactatagggagaggagttcgaatgccgctactacatt

SEQ ID NO: 458 - DNA, 48 bp, Artificial sequence
  Primer
taatacgactcactatagggagatcagacaagagctgggcaagaacat

SEQ ID NO: 459 - DNA, 52 bp, Artificial sequence
  Primer
taatacgactcactatagggagacgttctccgctttgtgcttggttttcgtg

SEQ ID NO: 460 - DNA, 53 bp, Artificial sequence
  Primer
taatacgactcactatagggagacgcttgtaggttttccgctggttgatgtcg

SEQ ID NO: 461 - DNA, 48 bp, Artificial sequence
  Primer
taatacgactcactatagggagaacgagaacatgccgacagtgggtaa

SEQ ID NO: 462 - DNA, 48 bp, Artificial sequence
  Primer
taatacgactcactatagggagagtttcggcaacggacttcctgttca

SEQ ID NO: 463 - DNA, 48 bp, Artificial sequence
  Primer
taatacgactcactatagggagaacgaggctgagataacaaactcatc

SEQ ID NO: 464 - DNA, 48 bp, Artificial sequence
  Primer
taatacgactcactatagggagactggaatatcatggccaaaccaaag

SEQ ID NO: 465 - DNA, 48 bp, Artificial sequence
  Primer
taatacgactcactatagggagatacaacgttcaggaatatgccaatc

SEQ ID NO: 466 - DNA, 48 bp, Artificial sequence
  Primer
taatacgactcactatagggagagtatatgcgttccacactcaacttt

SEQ ID NO: 467 - DNA, 47 bp, Artificial sequence
  Primer
taatacgactcactatagggagaccctgcaagagcgccattatcctg

SEQ ID NO: 468 - DNA, 48 bp, Artificial sequence
  Primer
taatacgactcactatagggagactctgtgtccggcatggctggttta

SEQ ID NO: 469 - DNA, 48 bp, Artificial sequence
  Primer
taatacgactcactatagggagatgactaccgctcaccaaactcaagt

SEQ ID NO: 470 - DNA, 48 bp, Artificial sequence
  Primer
taatacgactcactatagggagagggtccattatcattgcgatccagt

SEQ ID NO: 471 - DNA, 48 bp, Artificial sequence
  Primer
taatacgactcactatagggagaagttgcgaactgaaggtcaagtctc

SEQ ID NO: 472 - DNA, 48 bp, Artificial sequence
  Primer
taatacgactcactatagggagacattcgaaaccggccacatttagga

SEQ ID NO: 473 - DNA, 44 bp, Artificial sequence
  Primer
taatacgactcactatagggagaggctgccaaaagactgatgta

SEQ ID NO: 474 - DNA, 43 bp, Artificial sequence
  Primer
taatacgactcactatagggagaggaaggagaaccgcaagata

SEQ ID NO: 475 - DNA, 44 bp, Artificial sequence
  Primer
gcttctaatacgactcactatagtgcgtcaacacaactacaagg

SEQ ID NO: 476 - DNA, 44 bp, Artificial sequence
  Primer
gcttctaatacgactcactatagttgttcgcgacacatagtacg

SEQ ID NO: 477 - DNA, 49 bp, Artificial sequence
  Primer
taatacgactcactatagggagactttgcccagcttgtgtttcagttca

SEQ ID NO: 478 - DNA, 47 bp, Artificial sequence
  Primer
taatacgactcactatagggagaggcaatcctccacacccaccatcc

SEQ ID NO: 479 - DNA, 48 bp, Artificial sequence
  Primer
taatacgactcactatagggagaaaaggtggttatggcaaagtatttc

SEQ ID NO: 480 - DNA, 48 bp, Artificial sequence
  Primer
taatacgactcactatagggagaaaaatttcaggtgccatgtactcaa

SEQ ID NO: 481 - DNA, 48 bp, Artificial sequence
  Primer
taatacgactcactatagggagaattttgccgctgattggtggagttt

SEQ ID NO: 482 - DNA, 48 bp, Artificial sequence
  Primer
taatacgactcactatagggagacagcaggaataggagctatactatg

SEQ ID NO: 483 - DNA, 48 bp, Artificial sequence
  Primer
taatacgactcactatagggagacgctatatgaaccacatcgccagac

SEQ ID NO: 484 - DNA, 48 bp, Artificial sequence
  Primer
taatacgactcactatagggagaaacataaagggatggcagagaacag

SEQ ID NO: 485 - DNA, 47 bp, Artificial sequence
  Primer
taatacgactcactatagggagaatacgggaggaatttaagcaagtc

SEQ ID NO: 486 - DNA, 46 bp, Artificial sequence
  Primer
taatacgactcactatagggagattataacgcgtcggaagcagtct

SEQ ID NO: 487 - DNA, 50 bp, Artificial sequence
  Primer
taatacgactcactatagggagacgcgatgccgatggtcaggtgcaggag

SEQ ID NO: 488 - DNA, 48 bp, Artificial sequence
  Primer
taatacgactcactatagggagacctcgtccaatgcactcgatcaggg

SEQ ID NO: 489 - DNA, 48 bp, Artificial sequence
  Primer
taatacgactcactatagggagatgcagagtttattggcgaactggac

SEQ ID NO: 490 - DNA, 48 bp, Artificial sequence
  Primer
taatacgactcactatagggagaaagcttccagcatgcagacggatta

SEQ ID NO: 491 - DNA, 48 bp, Artificial sequence
  Primer
taatacgactcactatagggagaatgccaagccgaagaaccgactttt

SEQ ID NO: 492 - DNA, 48 bp, Artificial sequence
  Primer
taatacgactcactatagggagacatcatccacatcctcttgcacatc

SEQ ID NO: 493 - DNA, 48 bp, Artificial sequence
  Primer
taatacgactcactatagggagacagtagctcaatgttcaacactctg

SEQ ID NO: 494 - DNA, 48 bp, Artificial sequence
  Primer
taatacgactcactatagggagaatgaagatagctggccatctcactt

SEQ ID NO: 495 - DNA, 48 bp, Artificial sequence
  Primer
taatacgactcactatagggagaaccaccagtgcccgaaaagaaagtg

SEQ ID NO: 496 - DNA, 48 bp, Artificial sequence
  Primer
taatacgactcactatagggagaaccgaggcatctgtgacaagaaacc

SEQ ID NO: 497 - DNA, 44 bp, Artificial sequence
  Primer
gcttctaatacgactcactatagctcaccgtccattgcttctac

SEQ ID NO: 498 - DNA, 44 bp, Artificial sequence
  Primer
gcttctaatacgactcactataggcacaactgggactttagcat

SEQ ID NO: 499 - DNA, 58 bp, Artificial sequence
  Primer
taatacgactcactatagggagattaaaaagcgaaggaagcaaagagaaaacaacaaa

SEQ ID NO: 500 - DNA, 49 bp, Artificial sequence
  Primer
taatacgactcactatagggagatccaccagcccacatcgccagacacc

SEQ ID NO: 501 - DNA, 48 bp, Artificial sequence
  Primer
taatacgactcactatagggagacctgcagcgttgcttggagtgggat

SEQ ID NO: 502 - DNA, 48 bp, Artificial sequence
  Primer
taatacgactcactatagggagatttcctggtggccgctgacgagaca

SEQ ID NO: 503 - DNA, 48 bp, Artificial sequence
  Primer
taatacgactcactatagggagatgtgaagcacggcaagctgcaggag

SEQ ID NO: 504 - DNA, 48 bp, Artificial sequence
  Primer
taatacgactcactatagggagagtaggccgggaggtccttttggaac

SEQ ID NO: 505 - DNA, 48 bp, Artificial sequence
  Primer
taatacgactcactatagggagaagaaccgtggtacttccgcaaaatc

SEQ ID NO: 506 - DNA, 48 bp, Artificial sequence
  Primer
taatacgactcactatagggagacatgatctgggcttccgctaagaaa

SEQ ID NO: 507 - DNA, 48 bp, Artificial sequence
  Primer
taatacgactcactatagggagaaggagtacatgtccaagggcagtct

SEQ ID NO: 508 - DNA, 48 bp, Artificial sequence
  Primer
taatacgactcactatagggagagcactggagcagcagctgataaatg

SEQ ID NO: 509 - DNA, 48 bp, Artificial sequence
  Primer
taatacgactcactatagggagatcgaattcaacgctgccaacacctc

SEQ ID NO: 510 - DNA, 48 bp, Artificial sequence
  Primer
taatacgactcactatagggagagggcgtaaggaacgaagcgaatgac

SEQ ID NO: 511 - DNA, 48 bp, Artificial sequence
  Primer
taatacgactcactatagggagattcagtggtttaaggacagcattga

SEQ ID NO: 512 - DNA, 48 bp, Artificial sequence
  Primer
taatacgactcactatagggagacaggaagcatgaaatcttaaccttg

SEQ ID NO: 513 - DNA, 51 bp, Artificial sequence
  Primer
taatacgactcactatagggagagcggttcgggcctgcacagatttggtat

SEQ ID NO: 514 - DNA, 50 bp, Artificial sequence
  Primer
taatacgactcactatagggagatttgctgggcctttttgcttgatgctc

SEQ ID NO: 515 - DNA, 48 bp, Artificial sequence
  Primer
taatacgactcactatagggagacgacgtggaggcgaatggctttgat

SEQ ID NO: 516 - DNA, 53 bp, Artificial sequence
  Primer
taatacgactcactatagggagactgcttctgttcgcgctcggttcggtccat

SEQ ID NO: 517 - DNA, 44 bp, Artificial sequence
  Primer
gcttctaatacgactcactatagcagccgaaagcagtaattcat

SEQ ID NO: 518 - DNA, 45 bp, Artificial sequence
  Primer
gcttctaatacgactcactatagttgccttcattaatagccatgt

SEQ ID NO: 519 - DNA, 46 bp, Artificial sequence
  Primer
taatacgactcactatagggagattggtggggcagagaaagaggag

SEQ ID NO: 520 - DNA, 47 bp, Artificial sequence
  Primer
taatacgactcactatagggagagtcgccggcggtcgcattcaactg

SEQ ID NO: 521 - DNA, 48 bp, Artificial sequence
  Primer
taatacgactcactatagggagagaaccattgccaagcagattcagat

SEQ ID NO: 522 - DNA, 48 bp, Artificial sequence
  Primer
taatacgactcactatagggagatgaatgacatccagttccgagttgt

SEQ ID NO: 523 - DNA, 48 bp, Artificial sequence
  Primer
taatacgactcactatagggagacggtttgacgttggacaaggttcat

SEQ ID NO: 524 - DNA, 48 bp, Artificial sequence
  Primer
taatacgactcactatagggagacatctggttgctaaaacgagtggag

SEQ ID NO: 525 - DNA, 48 bp, Artificial sequence
  Primer
taatacgactcactatagggagaggcgcactcgaatgctttgaaaagg

SEQ ID NO: 526 - DNA, 48 bp, Artificial sequence
  Primer
taatacgactcactatagggagagctgacttggaagcgactgttagag

SEQ ID NO: 527 - DNA, 48 bp, Artificial sequence
  Primer
taatacgactcactatagggagacaagctcatccaacagcgttatctg

SEQ ID NO: 528 - DNA, 48 bp, Artificial sequence
  Primer
taatacgactcactatagggagaaagtagaaccgcttgagcttgaggt

SEQ ID NO: 529 - DNA, 48 bp, Artificial sequence
  Primer
taatacgactcactatagggagaagaactactacagcaacctggtgac

SEQ ID NO: 530 - DNA, 48 bp, Artificial sequence
  Primer
taatacgactcactatagggagagccgtctcactgatatagaactgtg

SEQ ID NO: 531 - DNA, 44 bp, Artificial sequence
  Primer
taatacgactcactatagggagaatgagcagcagaacgcaggac

SEQ ID NO: 532 - DNA, 43 bp, Artificial sequence
  Primer
taatacgactcactatagggagatcgcttcagccggagatact

SEQ ID NO: 533 - DNA, 48 bp, Artificial sequence
  Primer
taatacgactcactatagggagatcggatcagcatacatcacagagga

SEQ ID NO: 534 - DNA, 48 bp, Artificial sequence
  Primer
taatacgactcactatagggagagagaaaaggagccttacagcttgag

SEQ ID NO: 535 - DNA, 48 bp, Artificial sequence
  Primer
taatacgactcactatagggagagatagagggcctacgctatattcat

SEQ ID NO: 536 - DNA, 48 bp, Artificial sequence
  Primer
taatacgactcactatagggagaatatagactgcgaagtgggcctctt

SEQ ID NO: 537 - DNA, 47 bp, Artificial sequence
  Primer
taatacgactcactatagggagagcatcgggtacggccacattatta

SEQ ID NO: 538 - DNA, 47 bp, Artificial sequence
  Primer
taatacgactcactatagggagacgccgccttctttgcctatcttac

SEQ ID NO: 539 - DNA, 48 bp, Artificial sequence
  Primer
taatacgactcactatagggagacagatacctctagctgccttggaac

SEQ ID NO: 540 - DNA, 48 bp, Artificial sequence
  Primer
taatacgactcactatagggagacggaggtttatcgaggcgaggatta

SEQ ID NO: 541 - DNA, 48 bp, Artificial sequence
  Primer
taatacgactcactatagggagaaacagcaactgcaggccttgagggt

SEQ ID NO: 542 - DNA, 48 bp, Artificial sequence
  Primer
taatacgactcactatagggagaatacgtgcgctggcgatacgacttg

SEQ ID NO: 543 - DNA, 48 bp, Artificial sequence
  Primer
taatacgactcactatagggagatgggtcacctggttctgtggacttc

SEQ ID NO: 544 - DNA, 48 bp, Artificial sequence
  Primer
taatacgactcactatagggagagacgcttgggacgatattcgggatg

SEQ ID NO: 545 - DNA, 45 bp, Artificial sequence
  Primer
taatacgactcactatagggagacgccgccgagaaaacctacaac

SEQ ID NO: 546 - DNA, 42 bp, Artificial sequence
  Primer
taatacgactcactatagggagaccggcgccgtctcctcttc

SEQ ID NO: 547 - DNA, 47 bp, Artificial sequence
  Primer
taatacgactcactatagggagatgtcctctaacgccattgaaaact

SEQ ID NO: 548 - DNA, 45 bp, Artificial sequence
  Primer
taatacgactcactatagggagagtggcggcggcgtagaagaacc

SEQ ID NO: 549 - DNA, 44 bp, Artificial sequence
  Primer
taatacgactcactatagggagatatcgcgatcggagtgctaat

SEQ ID NO: 550 - DNA, 44 bp, Artificial sequence
  Primer
taatacgactcactatagggagagaccgctcgatcctcaaagta

SEQ ID NO: 551 - DNA, 52 bp, Artificial sequence
  Primer
taatacgactcactatagggagatgagactgcggcttgccttgattttccta

SEQ ID NO: 552 - DNA, 48 bp, Artificial sequence
  Primer
taatacgactcactatagggagatgagacgccggtttcgatggtgtgc

SEQ ID NO: 553 - DNA, 48 bp, Artificial sequence
  Primer
taatacgactcactatagggagacgtgccattctcttggactggttga

SEQ ID NO: 554 - DNA, 48 bp, Artificial sequence
  Primer
taatacgactcactatagggagactgccagcaccgagtaggaatagtt

SEQ ID NO: 555 - DNA, 47 bp, Artificial sequence
  Primer
taatacgactcactatagggagatgagagtgcggcgatccatagaat

SEQ ID NO: 556 - DNA, 47 bp, Artificial sequence
  Primer
taatacgactcactatagggagactccagacaccgatccgaatacaa